(19) United States
(12) Patent Application Publication
(10) Pub. No.: US 2004/0127549 A1
(43) Pub. Date: Jul. 1, 2004

(54) Cyclic 2-carbonylaminoketones as inhibitors of cruzipain and other cysteine proteases

(76) Inventor: Martin Quibell, Cambridge (GB)

Correspondence Address: HALE AND DORR, LLP, 60 STATE STREET, BOSTON, MA, 02109

(21) Appl. No.: 10/466,474

(22) PCT Filed: Jan. 17, 2002
(86) PCT No.: PCT/GB02/00190

(30) Foreign Application Priority Data
Jan. 17, 2001 (GB) 0101187.3

(51) Int. Cl. A61K 31/557

(52) U.S. Cl. 514/424; 514/573; 514/602; 514/613; 562/503; 564/86; 564/189

(57) ABSTRACT

Compounds of general formula (I), wherein R¹, R², R³, Y, (X)ₒ, (W)ₙ, (V)ₘ, Z and U are as defined in the specification, are inhibitors of cruzipain and other cysteine protease inhibitors and are useful as therapeutic agents, for example in Chagas' disease, or for validating therapeutic target compounds.

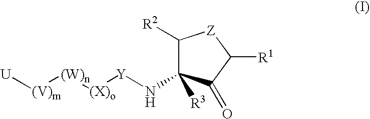

THIS INVENTION relates to compounds which are inhibitors of the protease cruzipain, a gene product of the Trypanosoma cruzi parasite. In particular, the invention provides compounds that are useful for the therapeutic treatment of Trypanosoma cruzi infection, to the use of these compounds, and to pharmaceutical compositions comprising them. Furthermore, this invention relates to compounds which are inhibitors across a broad range of cysteine proteases, to the use of these compounds, and to pharmaceutical compositions comprising them. Such compounds are useful for the therapeutic treatment of diseases in which participation of a cysteine protease is implicated.

The trypanosomal family of parasites have a substantial worldwide impact on human and animal healthcare (NcKerrow, J. H., et al, Ann. Rev. Microbiol. 4, 821-853, 1993). One parasite of this family, Trypanosoma cruzi, is the causative agent of Chagas' disease, which affects in excess of twenty million people annually in Latin and South America, is the leading cause of heart disease in these regions and results in more than 45,000 deaths per annum (Centers for Disease Control and prevention website). In addition, with the increase in migration of the infected population from rural to urban sites and movements from South and Central America into North America, the infection is spreading via blood transfusions, and at birth. The present treatments of choice for Trypanosoma cruzi infection, nifurtimox and benznidazole (an NADH fumarate reductase inhibitor, Turrens, J F, et al, Mol Biochem Parasitol., 82(1)125-9, 1996) are at best moderately successful, achieving 60% cure during the acute phase of infection (see Docampo, R. Curr. Pharm. Design, 7. 1157-1164, 2001 for a general discussion) whilst not being prescribed at all during the chronic phase where cardiomyopathy associated heart failure often occurs (Kirchhoff, L. V. New Engl. J. Med 329, 639-644, 1993). Additionally, these two drugs have serious adverse toxic effects, requiring close medical supervision during treatment, and have been shown to induce chromosomal damage in chagastic infants (Gorla, N. B. et al, Mutat. Res. 206, 217-220, 1988). Therefore, a strong medical need exists for new effective drugs for the chemotherapeutic treatment of Trypanosoma cruzi infection. Classically, the identification of enzymes found to be crucial for the establishment or propagation of an infectious disease has been instrumental in the development of successful drugs such as antivirals (e.g. HIV aspartyl protease inhibitors) and anti-bacterials (e.g. β-lactam antibiotics). The search for a similar Achilles heel in parasitic infections has examined numerous enzymes (e.g. parasitic dihydrofolate reductase, see Chowdhury, S. F. et al, J. Med. Chem., 42(21), 4300-4312, 1999; trypanothione reductase, see Li, Z. et al, Bioorg. Med. Chem. Lett., 11(2), 251-254, 2001; parasitic glyceraldehydes-3-phosphate dehydrogenase, see Aranov, A. M. et al, J. Med. Chem., 41(24), 4790-4799, 1998). A particularly promising area of research has identified the role of cysteine proteases, encoded by the parasite, that play a pivotal role during the life cycle of the parasite (McKerrow, 3. H., et al, Bioorg. Med. Chem., 7, 639-644, 1999). Proteases form a substantial group of biological molecules which to date constitute approximately 2% of all the gene products identified following analysis of several genome sequencing programmes (e.g. see Southan, C. J. Pept. Sci, 6. 453458, 2000). Proteases have evolved to participate in an enormous range of biological processes, mediating their effect by cleavage of peptide amide bonds within the myriad of proteins found in nature. This hydrolytic action is performed by initially recognising, then binding to, particular three-dimensional electronic surfaces displayed by a protein, which aligns the bond for cleavage precisely within the protease catalytic site. Catalytic hydrolysis then commences through nucleophilic attack of the amide bond to be cleaved either via an amino acid side-chain of the protease itself, or through the action of a water molecule that is bound to and activated by the protease. Proteases in which the attacking nucleophile is the thiol side-chain of a Cys residue are known as cysteine proteases. The general classification of ‘cysteine protease’ contains many members found across a wide range of organisms from viruses, bacteria, protozoa, plants and fingi to mammals.

Biological investigation of Trypanosoma cruzi infection has highlighted a number of specific enzymes that are crucial for the progression of the parasite's life cycle. One such enzyme, cruzipain, a cathepsin L-like cysteine protease, is a clear therapeutic target for the treatment of Chagas' disease ((a) Cazzulo, J. J. et al, Curr. Pharm. Des. 7, 1143-1156, 2001; (b) Caffrey, C. R. et al, Curr. Drug Targets, 1155-162, 2000). Although the precise biological role of cruzipain within the parasite's life cycle remains unclear, elevated cruzipain messenger RNA levels in the epimastigote developmental stage indicate a role in the nutritional degradation of host-molecules in lysosomal-like vesicles (Engel, J. C. et al, J. Cell. Sci, 11, 597-606, 1998). The validation of cruzipain as a viable therapeutic target has been achieved with increasing levels of complexity. Addition of a general cysteine protease inhibitor, Z-Phe-Ala-FMK to Trypanosoma cruzi-infected mammalian cell cultures blocked replication and differentiation of the parasite, thus arresting the parasite life cycle (Harth, G., et al, Mol. Biochem. Parasitol. 58, 17-24, 1993). Administration of a vinyl sulphone-based inhibitor in a Trypanosoma cruzi-infected murine animal model not only rescued the mice from lethal infections, but also produced a complete recovery (Engel, J. C. et al, J. Exp. Med, 188(4), 725-734, 1998). Numerous other in vivo studies have confirmed that infections with alternative parasites such as Leishmania major (Selzer, P. M. et al, Proc. Nat'l. Acad. Sci. U.S.A., 96, 11015-11022, 1999), Schistosoma mansoni and Plasmodium falciparium (Olson, J. E. et al, Bioorg. Med. Chem., 7, 633-638, 1999) can be halted or cured by treatment with cysteine protease inhibitors.

A variety of synthetic approaches have been described towards the design of cruzipain inhibitors. However, although providing a biological ‘proof-of-principle’ for the treatment of Trypanosoma cruzi infection, current inhibitors exhibit a number of biochemical and physical properties that may preclude their clinical development. (e.g. see (a) Brinen, L. S. et al, Structure, 8. 831-840, 2000, peptidomimetic vinyl sulphones, possible adverse mammalian cell toxicity (see McKerrow, J. H. and Engel, J. unpublished results cited in Scheidt, K. A. et al, Bioorg. Med. Chem, 6, 2477-2494, 1998); (b) Du, X Y et al, Chem. Biol., 7. 733-742, 2000, aryl ureas, generally with low 1M activity, and high ClogP values, thus poor aqueous solubility and probably low oral bioavailability, (c) Roush, W. R. et al, Tetrahedron, 56, 9747-9762, 2000, peptidyl epoxysuccinates, irreversible inhibitors, with potent activity verses house-keeping mammalian proteases such as cathepsin B; (d) Li, R. et al, Bioorg. Med Chem. 4(9), 1421-1427, 1996, bisarylacylhydrazides and chalcones, polyhydroxylated aromatics; (e) U.S. Pat. No. 6,143,931, WO 9846559, non-peptide α-ketoamides). Of the many different approaches to enzyme inhibition to date, only the cruzipain protease inhibitors have proven effective in curing disease-related animal models of Trypanosoma cruzi infection. Therefore, a clear medical need exists to progress these ‘proof-of-principle’ findings towards clinical candidates, suitable for human use, through the discovery of more efficacious cruzipain inhibitors that have a desirable combination of potency, selectivity, low toxicity and optimised phanmacokinetic parameters.

Cruzipain and indeed many other crucial parasitic proteases belong to the papain-like CA C1 family and have close structural mammalian homologues. Cysteine proteases are classified into ‘clans’ based upon a similarity in the three-dimensional structure or a conserved arrangement of catalytic residues within the protease primary sequence. Additionally, ‘clans’ are further classified into ‘families’ in which each protease shares a statistically significant relationship with other members when comparing the portions of amino acid sequence which constitute the parts responsible for the protease activity (see Barrett, A. J et al, in ‘Handbook of Proteolytic Enzymes’, Eds. Barrett, A. J., Rawlings, N. D., and Woessner, J. F. Publ. Academic Press, 1998, for a thorough discussion). To date, cysteine proteases have been classified into five clans, CA, CB, CC, CD and CE (Barrett, A. J. et al, 1998). A protease from the tropical papaya fruit ‘papain’ forms the foundation of clan CA, which currently contains over 80 distinct and complete entries in various sequence databases, with many more expected from the current genome sequencing efforts. Proteases of clan CA/family C1 have been implicated in a multitude of disease processes e.g. human proteases such as cathepsin K (osteoporosis), cathepsin S (autoimmune disorders), cathepsin L (metastases) or parasitic proteases such as falcipain (malaria parasite Plasmodium falciparum), cruzipain (Trypanosoma cruzi infection). Recntly a bacterial protease, staphylopain (S. aureus infection) has also been tentatively assigned to clan CA. X-ray crystallographic structures are available for a range of the above mentioned proteases in complex with a range of inhibitors e.g. papain (PDB entries, 1pad, 1pe6, 1pip, 1pop, 4pad, 5pad, 6pad, 1ppp, 1the, 1csb, 1huc), cathepsin K (lau0, lau2, lau3, lau4, latk, 1mem, 1bgo, 1ayw, 1ayu), cathepsin L (1cs8), cathepsin S (currently on-hold, but published McGrath, M. E. et al, Protein Science, 7, 1294-1302, 1998), cruzain (a recombinant form of cruzipain see Eakin, A. E. et al, 268(9), 6115-6118, 1993) (1ewp, 1aim, 2aim, 1F29, 1F2A, 1F2B, 1F2C), staphylopain (1cv8). Each of the structures displays a similar overall active-site topology, as would be expected by their ‘clan’ and ‘family’ classification and such structural similarity exemplifies one aspect of the difficulties involved in discovering a selective inhibitor of cruzipain suitable for human use. However, subtle differences in terms of the depth and intricate shape of the active site groove of each CA C1 protease are evident, which may be exploited for selective inhibitor design. Additionally, many of the current substrate-based inhibitor complexes of CA C1 family proteases show a series of conserved hydrogen bonds between the inhibitor and the protease backbone, which contribute significantly to inhibitor potency. Primarily a bidentate hydrogen-bond is observed between the protease Gly66 (C═O)/inhibitor N—H and the protease Gly66(NH)/inhibitor (C═O), where the inhibitor (C═O) and (NH) are provided by an amino acid residue NHCHRCO that constitutes the S2 sub-site binding element within the inhibitor (see Berger, A. and Schecter, I. Philos. Trans. R. Soc. Lond. [Biol.], 257, 249-264, 1970 for a description of protease binding site nomenclature). A further hydrogen-bond between the protease main-chain (C═O) of asparagine or aspartic acid (158 to 163, residue number varies between proteases) and an inhibitor (N—H) is often observed, where the inhibitor (N—H) is provided by the S1 sub-site binding element within the inhibitor. Thus, the motif X—NHCHRCO—NH—Y is widely observed amongst the prior art substrate-based inhibitors of CA C1 proteases.

In the prior art, the development of cysteine protease inhibitors for human use has recently been an area of intense activity. Considering the CA C1 family members, particular emphasis has been placed upon the development of inhibitors of human cathepsins, primarily cathepsin K (osteoporosis), cathepsin S (autoimmune disorders) and cathepsin L (metastases), through the use of peptide and peptidomimetic nitriles (e.g. see WO-A-0109910, WO-A-0051998, WO-A-0119816, WO-A-9924460, WO-A-0049008, WO-A-0048992, WO-A-0049007, WA-A-0130772, WO-A-0055125, WO-A-0055126, WO-A-0119808, WO-A-0149288, WO-A-0147886), linear and cyclic peptide and peptidomimetic ketones (e.g. see Veber, D. F. and Thompson, S. K., Curr. Opin. Drug Discovery Dev., 4, 362-369, 2000, WO-A-0170232, WO-A-0178734, WO-A-0009653, WO-A-0069855, WO-A-0029408, WO-A-0134153 to WO-A-0134160, WO-A-0029408, WO-A-9964399, WO-A-9805336, WO-A-9850533), ketoheterocycles (e.g. see WO-A-0055144, WO-A-0055124) and monobactams (e.g. see WO-A-0059881, WO-A-9948911, WO-A-0109169). The prior art describes potent in vitro inhibitors, but also highlights the many difficulties in developing a human therapeutic. For example, WO-A-9850533 and WO-A-0029408 describe compounds that may be referred to as cyclic ketones and are inhibitors of cysteine proteases with a particular reference towards papain family proteases and as a most preferred embodiment, cathepsin K. WO-A-9850533 describes compounds subsequently detailed in the literature as potent inhibitors of cathepsin K with good oral bioavailability (Witherington, J., ‘Tetrahydrofurans as Selective Cathepsin K Inhibitors’, RSC meeting, Burlington House, London, 1999). The compounds of WO-A-9850533 were reported to bind to cathepsin K through the formation of a reversible covalent bond between the tetrahydrofuran carbonyl and the active site catalytic cysteine residue (Witherington, J., 1999). Additionally, the same cyclic ketone compounds are described in WO-A-9953039 as part of a wide-ranging description of inhibitors of cysteine proteases associated with parasitic diseases, with particular reference to the treatment of malaria by inhibition of falcipain. However, subsequent literature describes the cyclic ketone compounds of WO-A-9850533 to be unsuitable for further development or for full pharmacokinetic evaluation due to a physiochemical property of the inhibitors, the poor chiral stability of the o-aminoketone chiral centre (Marquis, R. W. et al, J. Med. Chem., 44(5), 725-736, 2001). WO-A-0069855 describes compounds that may also be referred to as cyclic ketones with particular reference towards inhibition of cathepsin S. The compounds of WO-A-0069855 are considered to be an advance on compounds of WO-A-9850533 due to the presence of the β-substituent on the cyclic ketone ring system that provides increased chiral stability to the α-carbon of the cyclic ketone ring system. In an attempt to solve the problem of poor chiral integrity, subsequent literature has provided a closely related cyclic ketone series to that described in WO-A-9850533, where an approximately 300-fold loss in inhibitor potency was observed upon introduction of an alkyl group in place of the labile α-proton (Marquis, R. W. et al, J. Med. Chem, 4, 1380-1395, 2001). Additionally, subsequent literature has shown that within the cyclic ketone series described in WO-A-9850533, the α-(S) isomer is approximately 10 to 80-fold more potent than the α-(R) isomer (Fenwick, A. E. et al, Bioorg. Med. Chem. Lett., 1199-202, 2001).

It has now been discovered that certain compounds, defined by general formula (I), are potent and selective cruzipain protease inhibitors which are useful in the treatment of Trypanosoma cruzi infection. Other compounds defined by general formula (I) are protease inhibitors across a broad range of CA C1 cysteine proteases and compounds useful in the treatment of diseases caused by cysteine proteases. Compounds described by general formula (I) contain an α-alkyl group, of the R-stereo-configuration (or the S-stereo-configuration when Z=‘S’), yet surprisingly compounds defined by general formula (I) retain good potency. The present invention provides substituted (2-alkyl-3-alkyl-4-oxo-tetrahydrofuran-3-yl)amide, (2-alkyl-3-alkyl-4-oxo-tetrahydro-thiophen-3-yl)amide and (1-alkyl-2-alkyl-5-oxocyclopentyl)amide compounds defined by general formula (I).

Accordingly, the first aspect of the invention provides a compound according to formula (I):

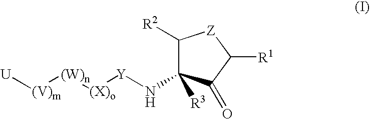

wherein:

R¹=C₀₋₇-alkyl (when C=0, R¹ is simply hydrogen), C₃₋₆-cycloalkyl or Ar—C₀₋₇-alkyl (when C=0, R¹ is simply an aromatic moiety Ar);

R²=C₀₋₇-alkyl, C₃₋₆-cycloalkyl or Ar—C₀₋₇-alkyl;

R³ C₁₋₇-alkyl, C₃₋₆-cycloalkyl or Ar—C₀₋₇-akyl;

Y═CR⁴R⁵—CO where R⁴, R⁵ are independently chosen from C₀₋₇-alkyl, C₃₋₆-cycloalkyl and Ar—C₀₋₇-alkyl;

(X)ₒ=CR⁶R⁷, where R⁶ and R⁷ are independently chosen from C₀₋₇-alkyl, C₃₋₆-cycloalkyl and Ar—C₀₋₇-alkyl and o is a number from zero to three;

(W)ₙ═O, S, C(O), S(O) or S(O)₂ or NR⁸, where R⁸ is chosen from C₀₋₇-alkyl, C₃₋₆-cycloallyl and Ar—C₀₋₇-alkyl and n is zero or one;

(V)ₘ═C(O), C(S), S(O), S(O)₂, S(O)₂NH, OC(O), NHC(O), NHS(O), NHS(O)₂, OC(O)NH, C(O)NH or CR⁹R¹⁰, where R⁹ and R¹⁰ are independently chosen from C₀₋₇-alkyl, C₃₋₆-cycloalkyl, Ar—C₀₋₇-alkyl and m is a number from zero to three, provided that when m is greater than one, (V)M contains a maximum of one carbonyl or sulphonyl group;

Z=0 (in which case compounds of general formula (I) may be named as (2-alkyl-3-alkyl-4-oxo-tetrahydrofuran-3-yl)amides, S (in which case compounds of general formula (I) may be named as (2-alkyl-3-alkyl-4-oxo-tethydrothiophen-3-yl)amides or CH₂ (in which case compounds of general formula (I) may be named as and (1-alkyl-2-alkyl-5-oxocyclopentyl)amides;

U=a stable 5- to 7-membered monocyclic or a stable 8- to 11-membered bicyclic ring which is either saturated or unsaturated and which includes zero to four heteroatoms (as detailed below):

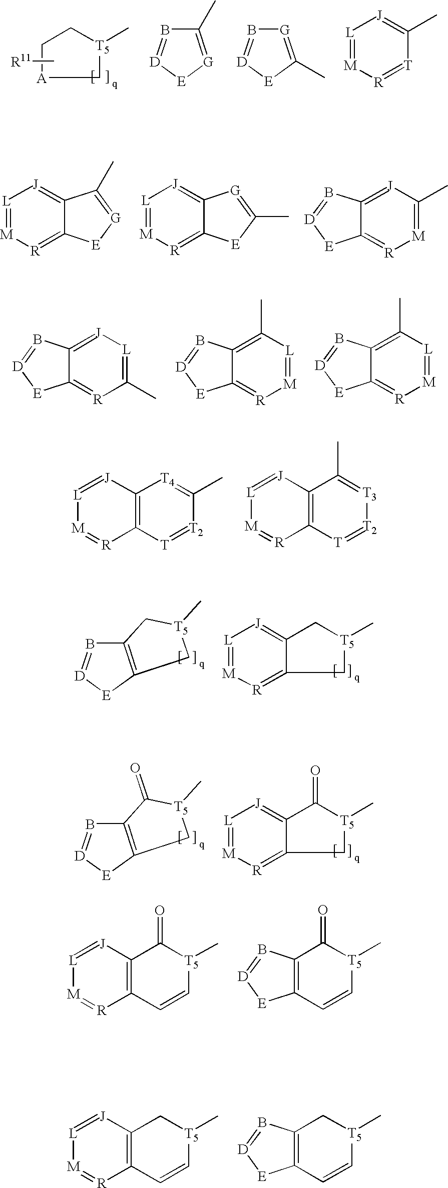

wherein R¹¹ is chosen from:

C₀₋₇-alkyl, C₃₋₆cycloalkyl, Ar—C₀₋₇-alkyl, halogen, O—C₀₋₇-alkyl, O—-C₃₋₄-cycloalkyl, O—Ar—C₀₋₇-alkyl, S—C₀₋₇-alkyl, S—C₃₋₄-cycloalkyl, S—Ar—C₀₋₇-alkyl, NH—C₀₋₇-alkyl, NH—C₃₋₆-cycloalkyl, NH—Ar—C₀₋₇-alkyl, N—(C₀₋₇-alkyl, N—(C₃₋₄-cycloalkyl)₂ and N—(Ar—C₀₋₇-alkyl)₂; or, when part of a group CHR¹¹ or CR¹¹, R¹¹ may be halogen;

A is chosen from:

CH₂, CYHR¹¹, O, S and NR¹², where R¹¹ is as defined above and where R¹² is chosen from C₀₋₇-alkyl, C₃₋₆-cycloalkyl and Ar—C₀₋₇-alkyl;

B, D and G are independently chosen from:

CR¹¹, where R¹¹ is as defined above, or N;

E is chosen from:

CH₂, CHR¹¹, O, S and NR¹², where R¹¹ and R¹² are defined as above;

J, L, M, R, T, T₂, T₃ and T₄ are independently chosen from:

CR¹¹ and N, where R¹¹ is as defined above;

T₅ is chosen from:

CH or N;

q is a number from one to three, thereby defining a 5-, 6- or 7-membered ring.

B, D, G, J, L, M, R, T, T₂, T₃ and T₄ may additionally represent an N-oxide (N→O).

The present invention includes all salts, hydrates, solvates, complexes and prodrugs of the compounds of this invention. The term “compound” is intended to include all such salts, hydrates, solvates, complexes and prodrugs, unless the context requires otherwise.

Appropriate pharmaceutically and veterinarily acceptable salts of the compounds of general formula (I) include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Salts which are not pharmaceutically or veterinarily acceptable may still be valuable as intermediates.

Prodrugs are any covalently bonded compounds which release the active parent drug according to general formula (I) in vivo. A prodrug may for example constitute an acetal or hemiacetal derivative of the exocyclic ketone functionality present in the (2-alkyl-3-alkyl-4-oxo-tetrahydrofuran-3-yl)amide, (2-alkyl-3-alkyl-4-oxo-tetrahydro-thiophen-3-yl)amide or (1-alkyl-2-alkyl-5-oxocyclopentyl)amide scaffold. If a chiral centre or another form of isomeric centre is present in a compound of the present invention, all forms of such isomer or isomers, including enantiomers and diastereoisomers, are intended to be covered herein. Compounds of the invention containing a chiral centre may be used as a racemic mixture, an enantiomerically enriched mixture, or the racemic mixture may be separated using well-known techniques and an individual enantiomer may be used alone.

‘Halogen’ as applied herein is meant to include F, Cl, Br, I;

‘Heteroatom’ as applied herein is meant to include O, S and N;

‘C₀₋₇-alkyl’ as applied herein is meant to include stable straight and branched chain aliphatic carbon chains containing zero (i.e. simply hydrogen) to seven carbon atoms such as methyl, ethyl, n-propyl, isopropyl, n-butyl, isobutyl, t-butyl, pentyl, isopentyl, hexyl, heptyl and any simple isomers thereof. Additionally, any C₀₋₇-alkyl may optionally be substituted at any point by one, two or three halogen atoms (as defined above) for example to give a trifluoromethyl substituent Furthermore, C₀₋₇-alkyl may contain one or more heteroatoms (as defined above) for example to give ethers, thioethers, sulphones, sulphonamides, substituted amines, amidines, guanidines, carboxylic acids, carboxamides. If the heteroatom is located at a chain terminus then it is appropriately substituted with one or two hydrogen atoms. A heteroatom or halogen is only present when C₀₋₇-alkyl contains a minimum of one carbon atom.

C₁₋₇-alkyl as applied herein is meant to include the definitions for C₀₋₇-alkyl (as defined above) but describes a substituent that comprises a minimum of one carbon.

‘C₃₋₄-cycloalkyl’ as applied herein is meant to include any variation of ‘C₀₋₇-alkyl’ which additionally contains a carbocyclic ring such as cyclopropyl, cyclobutyl, cyclopentyl, cyclohexyl. The carbocyclic ring may optionally be substituted with one or more halogens (as defined above) or heteroatoms (as defined above) for example to give a tetrahydrofuran, pyrrolidine, piperidine, piperazine or morpholine substituent.

‘Ar—C₄₋₇-alkyl’ as applied herein is meant to include any variation of C₀₋₇-alkyl which additionally contains an aromatic ring moiety ‘Ar’. The aromatic ring moiety Ar can be a stable 5 or 6-membered monocyclic or a stable 9 or 10 membered bicyclic ring which is unsaturated, as defined previously for U in general formula (I). The aromatic ring moiety Ar may be substituted by R¹ (as defined above for U in general formula (I)). When C=0 in the substituent Ar—C₀₋₇-allyl, the substituent is simply the aromatic ring moiety Ar.

Other expressions containing terms such as alkyl and cycloalkyl are intended to be construed according to the definitions above. For example “C₁₋₄ alkyl” is the same as C₀₋₇-alkyl except that it contains from one to four carbon atoms.

If different structural isomers are present, and/or one or more chiral centres are present, all isomeric forms are intended to be covered. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Preferred compounds of general formula (I) include those in which R¹ comprises C₀₋₇-alkyl or Ar—C₀₋₇-alkyl. Thus, for example, preferred R¹ moieties include hydrogen, or a straight or branched alkyl chain, or a straight or branched heteroalkyl chain, or an optionally substituted arylalkyl chain, or an optionally substituted arylheteroalkyl chain.

It is particularly preferred that R¹ is hydrogen or C₁₋₄ alkyl or Ar—C₁₋₄-alkyl and examples of such R¹ substituents include, but are not limited to:

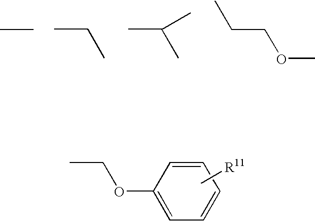

where R¹¹ is defined above.

It is preferred that R² is C₀₋₇-alkyl or Ar—C₀₋₇-alkyl, for example, hydrogen, straight or branched alkyl chains or heteroalkyl chains or optionally substituted arylalkyl chains. Particularly preferred compounds include those in which R² is C₀₋₄-alkyl or Ar—C₀₋₄-alkyl and examples of such R² substituents include, but are not limited to:

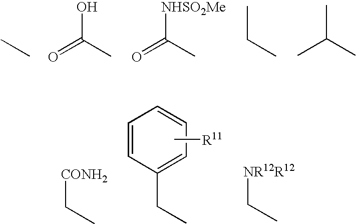

wherein R¹¹ and R¹² are as defined above.

Examples of (2-alkyl-3-alkyl-4-oxo-tetrahydrofuran-3-yl)amide scaffolds including such preferred R¹ and R² groups include:

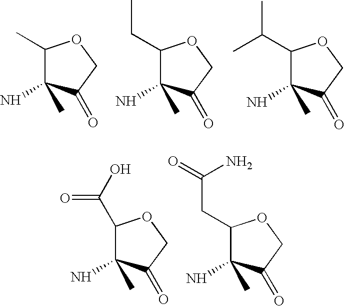

In preferred compounds of general formula (I), R³ is a simple alkyl or arylalkyl group such as methyl.

In preferred compounds of general formula (I), Y is CHR⁵CO where R⁵ is selected from C₀₋₇-alkyl or Ar—C₀₋₇-alkyl, for example hydrogen, a straight or branched alkyl chain, a straight or branched heteroalkyl chain, an optionally substituted arylalkyl chain or an optionally substituted arylheteroalkyl chain. Additionally, in preferred compounds of general formula (I), R⁵ is selected from C₃₋₆-cycloalkyl, for example cyclohexylmethyl.

Examples of preferred Y substituents include the following:

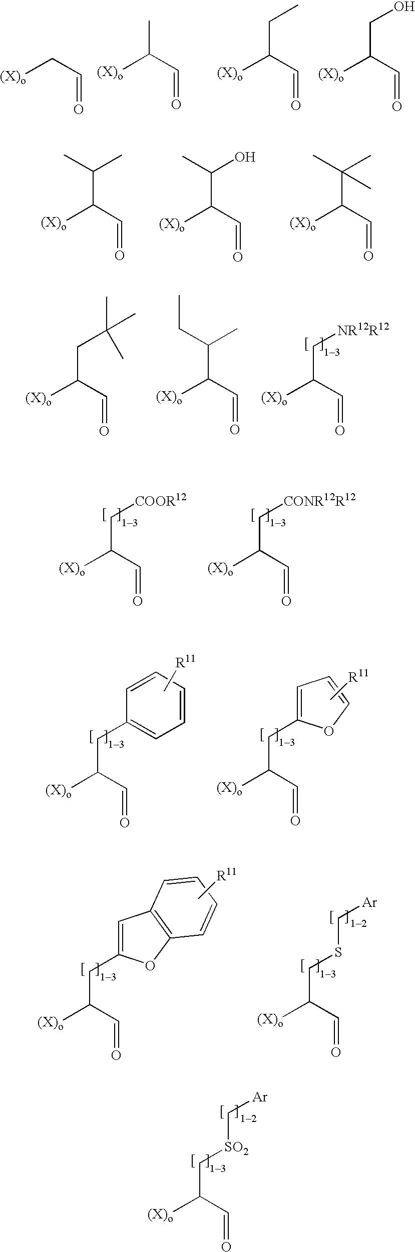

wherein R¹¹, R¹² and Ar are as defined above.

More preferred R⁵ groups include C₁₋₄-alkyl, which may be substituted with OH, NR¹²R¹², COOR¹², or CONR¹² or cycloalkylmethyl or Ar—C₁₋₄-alkyl, where the aryl group may be substituted with R¹¹; wherein each R¹¹ and R¹² is independently as defined above.

Even more preferred R⁵ groups comprise Ar—CH₂—, where the aromatic ring is an optionally substituted phenyl or monocyclic heterocycle. Additionally, even more preferred R⁵ groups comprise simple branched alkyl groups such as isobutyl or straight heteroalkyl chains such as benzylsulfanylmethyl or benzylsulphonylmethyl. Furthermore, even more preferred R⁵ groups comprise cyclohexylmethyl. Examples of even more preferred Y substituents comprise the following,

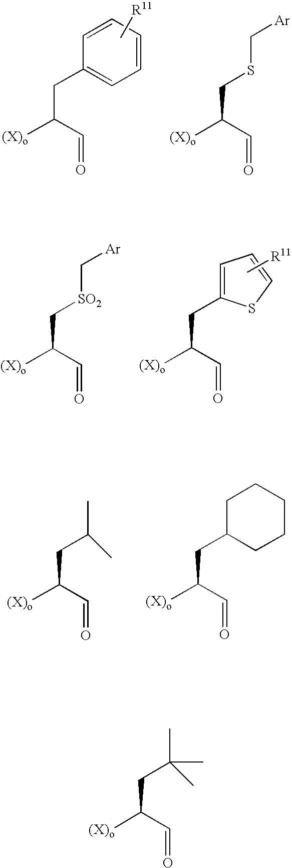

wherein R¹¹ and Ar are as defined previously

It is preferred that in the group (X)ₒ, each of R⁶ and R⁷ is selected from C₀₋₇-alkyl or Ar—C₀₋₇-alkyl, for example hydrogen, a straight or branched alkyl chain, a straight or branched heteroalkyl chain, an optionally substituted arylalkyl chain or an optionally substituted arylheteroalkyl chain.

More prefered (X)ₒ groups comprise R⁶ chosen form hydrogen; R⁷ is C₁₋₄-alkyl, which may be substituted with OH, NR¹²R¹², COOR¹², or CONR¹²; or Ar—C₁₋₄-alkyl, where the aryl group may be substituted with R¹¹, wherein each R¹¹ and R¹² is independently as defined above.

Examples of preferred (X)ₒ, groups include the following:

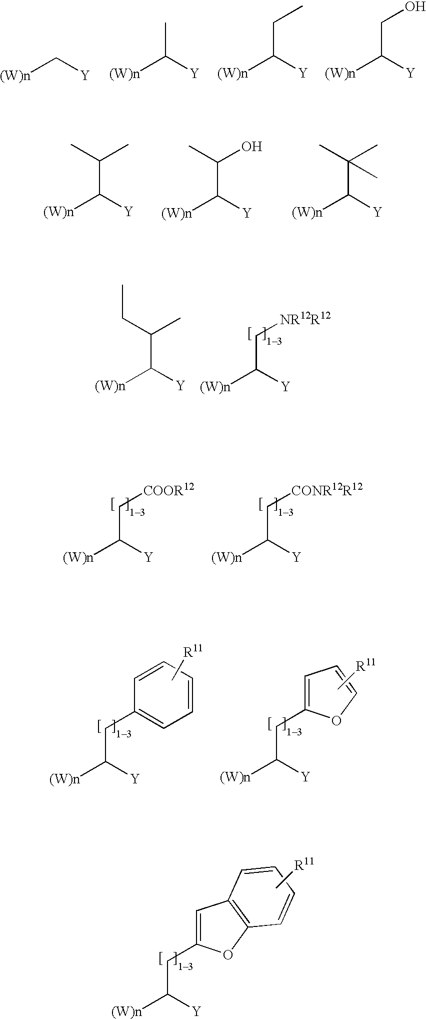

wherein R¹¹ and R¹² are as defined previously.

Even more preferred compounds of general formula (I), comprise (X)ₒ groups that are simple alkyl groups such as methylene and where o=0 or 1.

In the group (W)ₙ, W is preferably O, S, SO₂, S(O), C(O) or NR⁸, where R⁸ is C₀₋₄-alkyl; and n is 0 or 1.

More preferred compounds of general formula (I), comprise (W)ⁿ groups defined as O, S, SO₂, C(O) and NH where n=0 or 1.

In the group (V)ₘ:

V is preferably C(O), C(O)NH or CHR¹⁰, where R¹⁰ is C₀₋₄-alkyl; and

m is 0 or 1.

Preferred V and W substituent combinations encompassed by general formula (I) include, but are not limited to:

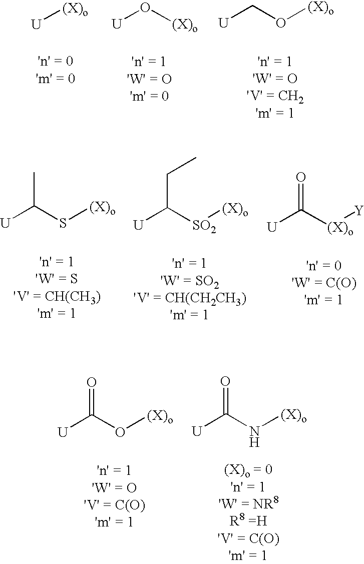

Additionally, a preferred V and W substituent combination encompassed by general formula (I) is:

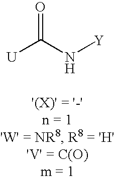

More preferred V, W and X substituent combinations encompassed by general formula (I) comprise, but are not limited to

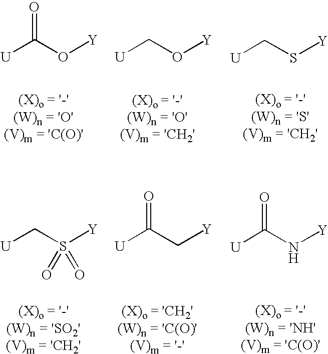

In preferred compounds of general formula (I), U comprises an optionally substituted 5- or 6-membered saturated or unsaturated heterocycle or Ar group or an optionally substituted saturated or unsaturated 9- or 10-membered heterocycle or Ar group. Examples of such preferred U rings include the following:

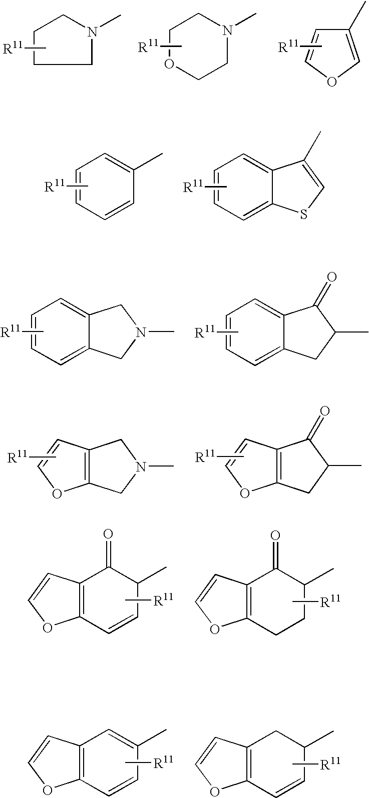

and also the following

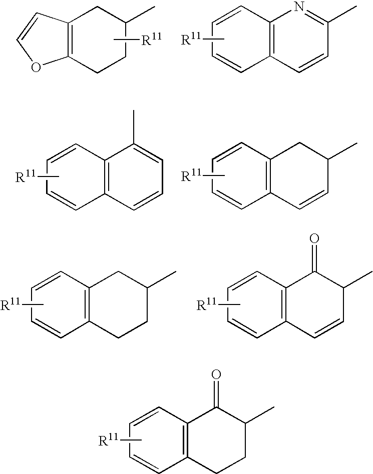

wherein R¹¹ is as defined previously.

More preferred compounds of general formula (I), contain a U group comprising of a bulky alkyl or aryl group at the para position of an aryl Ar. Also, more preferred compounds contain a meta or para-biaryl Ar—Ar, where Ar is as previously defined. Additionally, more preferred compounds contain a 6, 6 or 6, 5 or 5,6-fused aromatic ring. Examples of more preferred U groups are

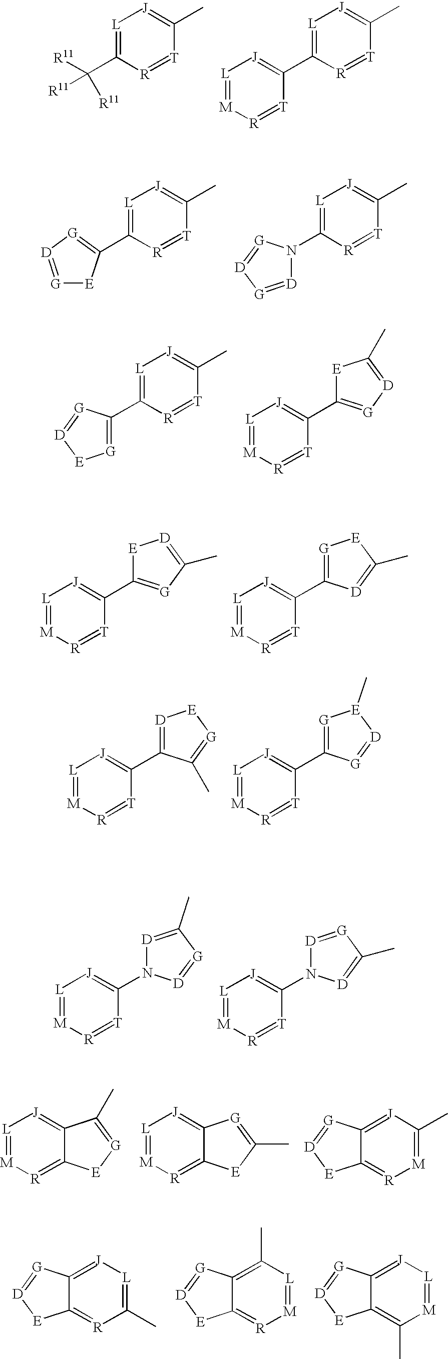

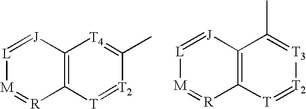

wherein R¹¹, D, E, G. J. L, M, R. T. T₂, T₃ and T₄ are as defined previously.

Even more preferred compounds of general formula (I), particularly for inhibition of cruzipain, contain a U group comprising a 6-membered Ar ring containing a bulky alkyl or aryl group at the para position, where Ar is as previously defined

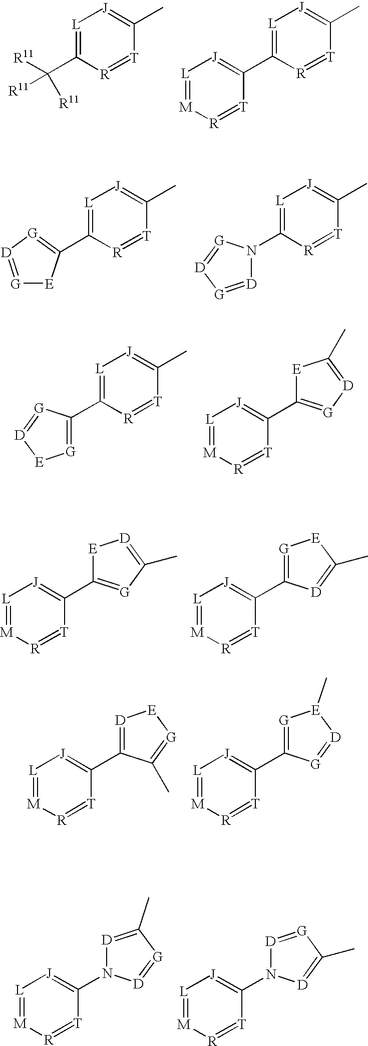

wherein R”, D, E, G. J. L, M, R and T are as defined previously.

Yet even more preferred compounds of general formula (I), contain a U group comprising but are not limited to the following,

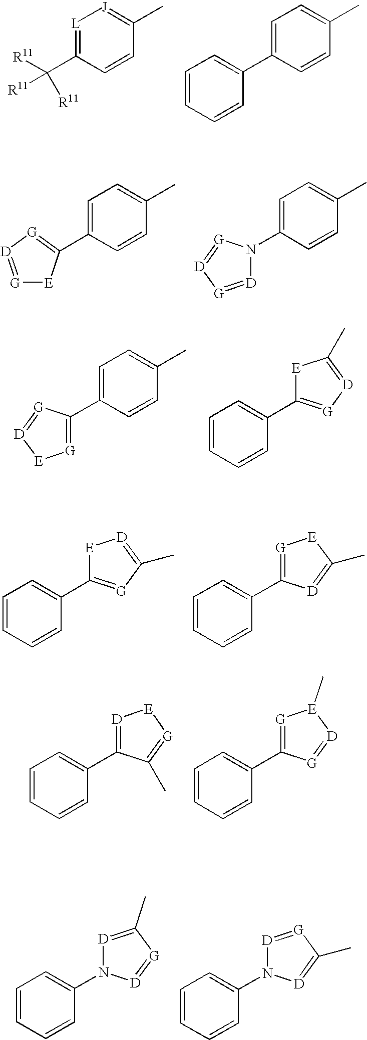

wherein R¹¹, D, E, G, J and L are as defined previously.

Abbreviations and symbols commonly used in the peptide and chemical arts are used herein to describe compounds of the present invention, following the general guidelines presented by the IUPAC-IUB Joint Commission on Biochemical Nomenclature as described in Eur. J. Biochem., 1589-, 1984. Compounds of formula (I) and the intermediates and starting materials used in their preparation are named in accordance with the IUPAC rules of nomenclature in which the characteristic groups have decreasing priority for citation as the principle group. An example compound of formula (I), compound (1) in which R¹ is H, R² is ethyl, R³ is methyl, Z is oxygen, Y is 4-methylpentyl, (X)₀ is zero, (W)ₙ is oxygen, (V)ₘ is methylene and U is phenyl is thus named:—

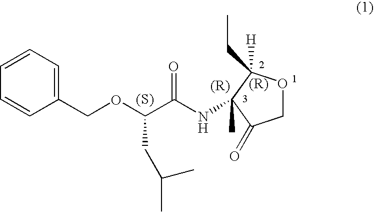

(2R, 3R) 2-Benzyloxy-4-methylpentanoic acid (2-ethyl-3-methyl-4oxo-tetrahydro fran-3-yl)amide

A second example compound of formula (I), compound (2) in which R¹ is H, R² is ethyl, R³ is methyl, Z is sulphur, Y is 4-methylpentyl, (X)₀ is zero, (W)ₙ is oxygen, (V)ₘ is methylene and U is phenyl is thus named:—

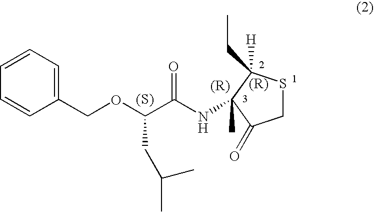

(2R, 3S) 2-Benzyloxy-4-methylpentanoic acid (2-ethyl-3-methyl4-oxo-tetrahydro thiophen-3-yl)amide

A third example compound of formula (I), compound (3) in which R¹ is H, R² is ethyl, R³ is methyl, Z is methylene, Y is 4-methylpentyl, (X)₀ is zero, (W)ₙ is oxygen, (V)ₘ is methylene and U is phenyl is thus named:—

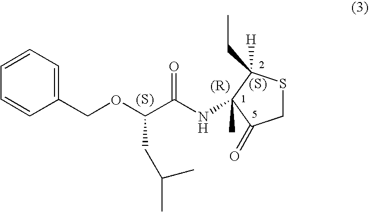

(1R, 28) 2-Benzyloxy-4-methylpentanoic acid (2-ethyl-1-methyl-5-oxo-cyclopentyl) amide

Compounds of the invention include, but are not limited to, the following examples that are the (2R, 3R) isomer of general formula (I) where Z=‘O’ and R¹=‘H’, and also include the equivalent analogues included in the full definition of Z and R′, R² and R³

N-[1-(2,3-Dimethyl-4-oxo-tetrahydrofuran-3-ylcarbamoyl)-2-(4-hydroxyphenyl)-ethyl]-benzamide

4-tert-Butyl-N-[1-(2,3-dimethyl-4-oxo-tetrahydrofuran-3-ylcarbamoyl)-2-(4-hydroxyphenyl)-ethyl]-benzamide

N-[1-(2,3-Dimethyl-4-oxo-tetrahydrofuran-3-ylcarbamoyl)-2-(4-hydroxyphenyl)-ethyl]-4-trifluoromethoxybenzamide

4-Dimethylamino-N-[1-(2,3-dimethyl-4-oxo-tetrahydrofuran-3-ylcarbamoyl)-2-(4-hydroxyphenyl)-ethyl]-benzamide

N-[1-(2,3-Dimethyl-4-oxo-tetrahydrofuran-3-ylcarbamoyl)-2-(4hydroxyphenyl)-ethyl]-4-isopropylbenzamide

4-Difluoromethoxy-N-[1-(2,3-dimethyl-4-oxo-tetrahydrofuran-3-ylcarbamoyl)-2-(4-hydroxyphenyl)-ethyl]-benzamide

N-[1-(2,3-Dimethyloxo-tetrahydrofuran-3-ylcarbamoyl)-2-(4-hydroxyphenyl)-ethyl]-4-trifluoromethylbenzamide

4-Bromo-N-[-(2,3-dimethyloxo-tetmhydrofuran-3-ylcarbamoyl)-2-(4 hydroxyphenyl)-ethyl]-benzamide

3-Bromo-N-[1-(2,3-ethyl-4-oxo-tetrahydrofuran-3-ylcarbamoyl)-2-(4-hydroxyphenyl)-ethyl]-benzamide

N-[12,3-Dimethyl-4-oxo-tetrahydrofuran-3-ylcarbamoyl)-2-(4-hydroxyphenyl)-ethyl]-4-vinylbenzamide

Naphthalene-2-caboxylic acid [1-(2,3-dimethyl-4-oxo-tetrahydrofuran-3-ylcarbamoyl)-2-(4-hydroxyphenyl)-ethyl]-amide

Naphthalene-1-carboxylic acid [1-(2,3-dimethyl-4-oxo-tetrahydrofuran-3-ylcarbamoyl)-2-(4-hydroxyphenyl)-ethyl]-amide

Quinoline-6-carboxylic acid [1-(2,3-dimethyloxo-tetrahydrofuran-3-ylcarbamoyl)-2-(4-hydroxyphenyl)-ethyl]-amide

Benzo[b]thiophene-2-carboxylic acid [1-(2,3-dimethyl-4-oxo-tetrahydrofuran-3-ylcarbamoyl)-2-(4hydroxyphenyl)-ethyl]-amide

Benzo[b]thiophene-3-carboxylic acid [1-(2,3-dimethyloxo-tetrahydrofuran-3-ylcarbamoyl)-2-(4-hydroxyphenyl)-ethyl]-amide

Benzothiazole-5-carboxylic acid [1-(2,3-dimethyl-4-oxo-tetrahydrofuran-3-ylcarbamoyl)-2-(4-hydroxyphenyl)-ethyl]-amide

Biphenylcarboxylic acid [1-(2,3-dimethyl-4-oxo-tetrahydrofuran-3-ylcarbamoyl)-2-(4-hydroxyphenyl)-ethyl]-amide

N-[1-(2,3-Dimethyl-4oxo-tetrahydrofuran-3-ylcarbamoyl)-2-(4-hydroxyphenyl)-ethy]-4-imidazol-1-ylbenzamide

N-[1-(2,3-Dimethyl-4-oxo-tetrahydrofuran-3-ylcarbamoyl)-2-(4-hydroxyphenyl)-ethyl]-4thiophen-2-ylbenzamide

N-[1-(2,3-Dimethyl-4-oxo-tetrahydrofuran-3-ylcarbamoyl)-2-(4-hydroxyphenyl) ethyl]oxazol-5-ylbenzamide

N-[1-(2,3-Dimethyl-4-oxo-tetrahydrofuran-3-ylcarbamoyl)-2-(4-hydroxyphenyl)-ethyl]-4-[1,2,3]thiadiazol-5-ylbenzamide

N-[1-(2,3-Dimethyl-4-oxo-tetrahydrofuran-3-ylcarbamoyl)-2-(4-hydroxyphenyl)-ethyl]-4-pyrazol-1-ylbenzamide

N-[1-(2,3-Dimethyl-4-oxo-tetrahydrofuran-3-ylcarbamoyl)-2-(4hydroxyphenyl)-ethyl]-5-thiophen-2-ylnicotinamide

2-Phenylthiazole-4carboxylic acid [1-(2,3-dimethyl-4-oxo-tetrahydrofuran-3-ylcarbamoyl)-2-(4-hydroxyphenyl)-ethyl]-amide

2-Pyridin-3-ylthiazole-4carboxylic acid [1-(2,3-dimethyl-4-oxo-tetrahydrofuran-3-ylcarbamoyl)-2-(4-hydroxyphenyl)-ethyl]-amide

5-Phenylthiophene-2-carboxylic acid [1-(2,3-dimethyl-4-oxo-tetrahydrofuran-3-ylcarbamoyl)-2-(4hydroxyphenyl)-ethyl]-amide

5-Pyridin-3-ylthiophene-2-carboxylic acid [1-(2,3-dimethyl-4-oxo-tetrahydrofuran-3-ylcarbamoyl)-2-(4-hydroxyphenyl)-ethyl]-amide

2-Methyl-5-phenylfuran-3-carboxylic acid [1-(2,3-dimethyl-4-oxo-tetrahydrofuran-3-ylcarbamoyl)-2-(4hydroxyphenyl)-ethyl]-amide

4-Phenylthiophene-2-carboxylic acid [1-(2,3-dimethyl-4-oxo-tetrahydrofuran-3-ylcarbamoyl)-2-(4hydroxyphenyl)ethyl]-amide

4-Pyridin-3-ylthiophene-2carboxylic acid [1(2,3-dimethyl-4-oxo-tetrahydrofuran-3-ylcarbamoyl)-2-(4-hydroxyphenyl)ethyl]-amide

2-Thiophen-2-ylthiazol-4-carboxylic acid [1-(2,3-dimethyloxo-tetrahydrofuran-3-ylcarbamoyl)-2-(4-hydroxyphenyl)-ethyl]-amide

3-Phenylpyrrole-1-carboxylic acid [1-(2,3-dimethyl-4-oxo-tetrahydrofuran-3-ylcarbamoyl)-2-(4hydroxyphenyl)-ethyl]-amide

N-[1-(2,3-Dimethyl-4-oxo-tetrahydrofuran-3-ylcarbamoyl)-3-methyl-butyl]benzamide

4-tert-Butyl-N-[1-(2,3-dimethyl-4-oxo-tetrahydrofuran-3-ylcarbamoyl)-3-methyl-butyl]-benzamide

N-[1-(2,3-Dimethyl-4-oxo-tetrahydrofuran-3-ylcarbamoyl)-3-methyl-butyl]-4-trifluoromethoxybenzamide

4-Dimethylamino-N-[1-(2,3-dimethyl-4-oxo-tetrahydrofuran-3-ylcarbamoyl)-3-methyl-butyl]-benzamide

4-Isopropyl-N-[1-(2,3-dimethyl-4-oxo-tetrahydrofuran-3-ylcarbamoyl)-3-methyl-butyl]-benzamide

4-Difluoromethoxy-N-[1-(2,3-dimethyloxo-tetrahydrofuran-3-ylcarbamoyl)-3-methyl-butyl]-benzamide

N-[1-(2,3-Dimethyloxo-tetrahydrofuran-3-ylcarbamoyl)-3-methyl-butyl]-4-trifluoromethylbenzamide

4-Bromo-N-[1-(2,3-dimethyl-4-oxo-tetrahydrofuran-3-ylcarbamoyl)-3-methyl-butyl]-benzamide

3-Bromo-N-[1-(2,3-dimethyl-4-oxo-tetrahydrofuran-3-ylcarbamoyl)-3-methyl-butyl]-benzamide

N-[1-(2,3-Dimethyl-4-oxo-tetrahydrofuran-3-ylcarbamoyl)-3-methyl-butyl]4-vinyl benzamide

Naphthalene-2-carboxylic acid [1-(2,3-dimethyl-4-oxo-tetrahydrofuran-3-ylcarbamoyl)-3-methyl-butyl]-amide

Naphthalene-1-carboxylic acid [1-(2,3-dimethyl-4-oxo-tetrahydrofuran-3-ylcarbamoyl)-3-methyl-butyl]-amide

Quinoline-6-carboxylic acid [1-(2,3-dimethyl-4-oxo-tetrahydrofuran-3-ylcarbamoyl)-3-methyl-butyl]-amide

Benzo[b]thiophene-2-carboxylic acid [1-(2,3-dimethyl-4-oxo-tetrahydrofuran-3-ylcarbamoyl)-3-methyl-butyl]-amide

Benzo[b]thiophene-3-carboxylic acid [1-(2,3-dimethyloxo-tetrahydrofuran-3-ylcarbamoyl)-3-methyl-butyl]-amide

Benzothiazole-5-carboxylic acid [1-(2,3-dimethyl-4-oxo-tetrahydrofuran-3-ylcarbamoyl)-3-methyl-butyl]-amide

Biphenyl-4carboxylic acid [1-(2,3-dimethyl-4-oxo-tetrahydrofuran-3-ylcarbamoyl)-3-methyl-butyl]-amide

4-imidazol-1-yl-N-[1-(2,3-Dimethyl-4-oxo-tetrahydrofuran-3-ylcarbamoyl)-3-methyl-butyl]benzamide

N-[1-(2,3-Dimethyl-4-oxo-tetrahydrofuran-3-ylcarbamoyl)-3-methyl-buty]-4-thiophen-2-ylbenzamide

N-[1-(2,3-Dimethyl-4-oxo-tetrahydrofuran-3-ylcarbamoyl)-3-methyl-butyl]4 oxazol-5-ylbenzamide

N-[1-(2,3-Dimethyl-4-oxo-tetrahydrofuran-3-ylcarbamoyl)-3-methyl-butyl]4[1,2,3]-thiadiazol-4-ylbenzamide

N-[1-(2,3-Dimethyl-4-oxo-tetrahydrofuran-3-ylcarbamoyl)-3-methyl-butyl]4-pyrazol-1-ylbenzamide

N-[1-(2,3-Dimethyl-4-oxo-tetrahydrofuran-3-ylcarbamoyl)-3-methyl-butyl]-5-thiophen-2-ylnicotinamide

2-Phenylthiazole-4-carboxylic acid [1-(2,3-dimethyl-4-oxo-tetrahydrofuran-3-ylcarbamoyl)-3-methyl-butyl]-amide

2-Pyridin-3-ylthiazole-4carboxylic acid [1-(2,3-dimethyl-4-oxo-tetrahydrofuran-3-ylcarbamoyl)-3-methyl-butyl]-amide

5-Phenylthiophene-2-carboxylic acid [[1-(2,3-dimethyl-4-oxo-tetrahydrofuran-3-ylcarbamoyl)-3-methyl-butyl]-amide

5-Pyridin-3-ylthiophene-2-carboxylic acid [1-(2,3-dimethyl-4-oxo-tetrahydrofuran-3-ylcarbamoyl)-3-methyl-butyl]-amide

2-Methyl-5-phenylfuran-3-carboxylic acid [1-(2,3-dimethyl-4-oxo-tetrahydrofuran-3-ylcarbamoyl)-3-methyl-butyl]-amide

4-Phenylthiophene-2-carboxylic acid [1-(2,3-dimethyl-4-oxo-tetrahydrofuran-3-ylcarbamoyl)-3-methyl-butyl]-amide

4-Pyridin-3-ylthiophene-2-carboxylic acid [1-2,3-dimethyloxo-tetrahydrofuran-3-ylcarbamoyl)-3-methyl-butyl]-amide

2-Thiophen-2-ylthiazole-4-carboxylic acid [1-(2,3-dimethyl-4-oxo-tetrahydrofuran-3-ylcarbamoyl)-3-methyl-butyl]-amide

3-Phenylpyrrole-1-carboxylic acid [1-(2,3-dimethyl-4-oxo-tetrahydrofuran-3-ylcarbamoyl)-3-methyl-butyl]-amide

N-[1-(2,3-Dimethyl-4-oxo-tetrahydrofuran-3-ylcarbamoyl)-2-(3-hydroxyphenyl)-ethyl]-benzamide

4-tert-Butyl-N-[1-(2,3-dimethyl-4-oxo-tetrahydrofuran-3-ylcarbamoyl)-2-(3-hydroxyphenyl)-ethyl]-benzamide

N-[1-(2,3-Dimethyl-4-oxo-tetrahydrofuran-3-ylcarbamoyl)-2-(3-hydroxyphenyl)-ethyl]-4-trifluoromethoxybenzamide

4-Dimethylamino-N-[1-(2,3-dimethyloxo-tetrahydrofuran-3-ylcarbamoyl)-2-(3-hydroxyphenyl)-ethyl]-benzamide

N-[1-(2,3-Dimethyloxo-tetrahydrofuran-3-ylcarbamoyl)-2-(3-hydroxyphenyl)-ethyl]4-isopropylbenzamide

4-Difluoromethoxy-N-[1-(2,3-dimethyl-4-oxo-tetrahydrofuran-3-ylcarbamoyl)-2-(3-hydroxyphenyl)-ethyl]-benzamide

N-[1-(2,3-Dimethyl4-oxo-tetrahydrofuran-3-ylcarbamoyl)-2-(3-hydroxyphenyl) ethyl]-4-trifluoromethylbenzamide

4-Bromo-N-[1-(2,3-dimethyl-4-oxo-tetrahydrofuran-3-ylcarbamoyl)-2-(3-hydroxyphenyl)ethyl]-benzamide

3-Bromo-N-[1-(2,3-dimethyl-4-oxo-tetrahydrofuran-3-ylcarbamoyl)-2-(3-hydroxyphenyl)-ethyl]-benzamide

N-[1-(2,3-Dimethyl-4oxo-tetrahydrofuran-3-ylcarbamoyl)-2-(3-hydroxyphenyl)-ethyl]-4-vinylbenzamide

Naphthalene-2-carboxylic acid [1-(2,3-dimethyl-4-oxo-tetrahydrofuran-3-ylcarbamoyl)-2-(3-hydroxyphenyl)-ethyl]-amide

Naphthalene-1-carboxylic acid [1-(2,3-dimethyl-4-oxo-tetrahydrofuran-3-ylcarbamoyl)-2-(3-hydroxyphenyl)-ethyl]-amide

Quinoline-6-carboxylic acid [1-(2,3-dimethyl-4-oxo-tetrahydrofuran-3-ylcarbamoyl)-2-(3-hydroxyphenyl)-ethyl]-amide

Benzo[b]thiophene-2-carboxylic acid [1-(2,3-dimethyl-4-oxo-tetrahydrofuran-3-ylcarbamoyl)-2-(3-hydroxyphenyl)-ethyl]-amide

Benzo[b]thiophene-3-carboxylic acid [1-(2,3-dimethyl-4-oxo-tetrahydrofuran-3-ylcarbamoyl)-2-(3-hydroxyphenyl)-ethyl]-amide

Benzothiazole-5-carboxylic acid [1-(2,3-dimethyl-4-oxo-tetrahydrofuran-3-ylcarbamoyl)-2-(3-hydroxyphenyl)-ethyl]-amide

Biphenyl-4-carboxylic acid [1-(2,3-dimethyl-4-oxo-tetrahydrofuran-3-ylcarbamoyl)-2-(3-hydroxyphenyl)-ethyl]-amide

N-[1-(2,3-Dimethyl-4-oxo-tetrahydrofuran-3-ylcarbamoyl)-2-(3-hydroxyphenyl)-ethyl]-4-imidazol-1-ylbenzamide

N-[1-(2,3-Dimethyl-4-oxo-tetrahydrofuran-3-ylcarbamoyl)-2-(3-hydroxyphenyl)-ethyl]-4-thiophen-2-ylbenzamide

N-[1-(2,3-Dimethyloxo-tetrahydrofuran-3-ylcarbamoyl)-2-(3-hydroxyphenyl)-ethyl]-4-oxazol-5-ylbenzamide

N-[1-(2,3-Dimethyloxo-tetrahydrofuran-3-ylcarbamoyl)-2-(3-hydroxyphenyl)-ethyl]-4-[1,2,3]thiadiazol-5-ylbenzamide

N-[1-(2,3-Dimethyl-4-oxo-tetrahydrofuran-3-ylcarbamoyl)-2-(3-hydroxyphenyl)-ethyl]-4-pyrazol-1-ylbenzamide

N-[1-(2,3-Dimethyl-4-oxo-tetrahydrofuran-3-ylcarbamoyl)-2-(3-hydroxyphenyl)-ethyl]-5-thiophen-2-ylnicotinamide

2-Phenylthiazole-4-carboxylic acid [1-(2,3-dimethyl-4-oxo-tetrahydrofuran-3-ylcarbamoyl)-2-(3-hydroxyphenyl)-ethyl]-amide

2-Pyridin-3-ylthiazole-4carboxylic acid [1-(2,3-dimethyl-4-oxo-tetrahydrofuran-3-ylcarbamoyl)-2-(3-hydroxyphenyl)-ethyl]-amide

5-Phenylthiophene-2-carboxylic acid [1-(2,3-dimethyl-4-oxo-tetrahydrofuran-3-ylcarbamoyl)-2-(3-hydroxyphenyl)-ethyl]-amide

5-Pyridin-3-ylthiophene-2-carboxylic acid [1-(2,3-dimethyl-4-oxo-tetrahydrofuran-3-ylcarbamoyl)-2-(3-hydroxyphenyl)-ethyl]-amide

2-Methyl-5-phenylfuran-3-carboxylic acid [1-(2,3-dimethyl-4oxo-tetrahydrofuran-3-ylcarbamoyl)-2-(3-hydroxyphenyl)-ethyl]-amide

4Phenylthiophene-2-carboxylic acid [1-(2,3-dimethyl-4-oxo-tetrahydrofuran-3-ylcarbamoyl)-2-(3-hydroxyphenyl)-ethyl]-amide

4-Pyridin-3-ylthiophene-2-carboxylic acid [1-(2,3-dimethyl-4-oxo-tetrahydrofuran-3-ylcarbamoyl)-2-(3-hydroxyphenyl)-ethyl]-amide

2-Thiophen-2-ylthiazolecarboxylic acid [1-(2,3-dimethyloxo-tetrahydrofuran-3-ylcarbamoyl)-2-(3-hydroxyphenyl)-ethyl]-amide 3-Phenylpyrrole-1-carboxylic acid [1-(2,3-dimethyloxo-tetrahydrofuran-3-ylcarbamoyl)-2-(3-hydroxyphenyl)-ethyl]-amide

Additional compounds of the invention include, but are not limited to, the following examples that are the (2R, 3R) isomer of general formula (I) where Z=‘O’ and R¹=‘H’, and also include the equivalent analogues included in the full definition of Z and R′, R² and R³

Furan-2-carboxylic acid [1-(2,3-dimethyl-4-oxo-tetrahydrofuran-3-ylcarbanoyl)-2-(4-hydroxyphenyl)-ethyl]-amide

Furan-3-carboxylic acid [1-(2,3-dimethyl-4-oxo-tetrahydrofuran-3-ylcarbamoyl)-2-(4-hydroxyphenyl)-ethyl]-amide

Thiophene-2-carboxylic acid [1-(2,3-dimethyl-4-oxo-tetrahydrofuran-3-ylcarbamoyl)-2-(4-hydroxyphenyl)-ethyl]-amide

Thiophene-3-carboxylic acid [1-(2,3-dimethyl-4-oxo-tetrahydrofuran-3-ylcarbamoyl)-2-(4-hydroxyphenyl)-ethyl]-amide

Morphohine-4-carboxylic acid [1-(2,3-dimethyl-4-oxo-tetrahydrofuran-3-ylcarbamoyl)-2-(4-hydroxyphenyl)-ethyl]-amide

Furan-2-carboxylic acid [1-(2,3-dimethyl-4-oxo-tetrahydrofuran-3-ylcarbamoyl)-3-methyl-butyl]-amide

Furan-3-carboxylic acid [1-(2,3-dimethyl-4-oxo-tetrahydrofuran-3-ylcarbamoyl)-3-methyl-butyl]-amide

Thiophene-2-carboxylic acid [1-(2,3-dimethyl-4-oxo-tetrahydrofuran-3-ylcarbamoyl)-3-methyl-butyl]-amide

Thiophene-3-carboxylic acid [1-(2,3-dimethyl-4-oxo-tetrahydrofuran-3-ylcarbamoyl)-3-methyl-butyl]-amide

Morpholine-4-carboxylic acid [1-(2,3-dimethyl-4-oxo-tetrahydrofuran-3-ylcarbamoyl)-3-methyl-butyl]-amide

Furan-2-carboxylic acid [2-cyclohexyl-1-(2,3-dimethyl-4-oxo-tetrahydrofuran-3-ylcarbamoyl)-ethyl]-amide

Furan-3-carboxylic acid [2-cyclohexyl-1-(2,3-dimethyloxo-tetrahydrofuran-3-ylcarbamoyl)-ethyl]-amide

Thiophene-2-carboxylic acid [2-cyclohexyl-1-(2,3-dimethyl-4-oxo-tetrahydrofuran-3-ylcarbamoyl)-ethyl]-amide

Thiophene-3carboxylic acid [2-cyclohexyl-1-(2,3-dimethyl-4-oxo-tetrahydrofuran-3-ylcarbamoyl)-ethyl]-amide

Morpholine-4-carboxylic acid [2-cyclohexyl-1-(2,3-dimethyl-4-oxo-tetrahydrofuran-3-ylcarbamoyl)-ethyl]-amide

Naphthalene-1-carboxylic acid [2-cyclohexyl-1-(2,3-dimethyl-4-oxo-tetrahydrofuran-3-ylcarbamoyl)ethyl]-amide

Naphthalene-2-carboxylic acid [[2-cyclohexyl-1-(2,3-dimethyl-4-oxo-tetrahydrofuran-3-ylcarbamoyl)-ethyl]-amide

Benzo[b]thiophene-2-carboxylic acid [2-cyclohexyl-1-(2,3-dimethyl-4-oxo-tetrahydrofuran-3-ylcarbamoyl)-ethyl)-amide

Furan-2-carboxylic acid [1-(2,3-dimethyloxo-tetrahydrofuran-3-ylcarbamoyl)-3,3-dimethyl-butyl]-amide

Furan-3-carboxylic acid 1-(2,3-dimethyl-4-oxo-tetrahydrofuran-3-ylcarbamoyl)-3,3-dimethyl-butyl]-amide

Thiophene-2-carboxylic acid 1-(2,3-dimethyl-4-oxo-tetrahydrofuran-3-ylcarbamoyl)-3,3-dimethyl-butyl]-amide

Thiophene-3-carboxylic acid 1-(2,3-dimethyl-4-oxo-tetrahydrofuran-3-ylcarbamoyl)-3,3-dimethyl-butyl]-amide

Morpholine-4-carboxylic acid 1-(2,3-dimethyloxo-tetrahydrofuran-3-ylcarbamoyl)-3,3dimethyl-butyl]-amide

Benzo[b]thiophene-2-carboxylic acid 1-(2,3-dimethyl-4-oxo-tetrahydrofuran-3-ylcarbamoyl)-3,3-dimethyl-butyl]-amide

Naphthalene-1-carboxylic acid 1-(2,3-dimethyl-4-oxo-tetrahydrofuran-3-ylcarbamoyl)-3,3-dimethyl-butyl]-amide

Naphthalene-2-carboxylic acid 1-(2,3-dimethyl-4-oxo-tetrahydrofuran-3-ylcarbamoyl)3,3-dimethyl-butyl]-amide

N-[1-(2,3-Dimethyl-4-oxo-tetrahydrofuran-3-ylcarbamoyl)-3,3-dimethyl-butyl]-benzamide

Furan-2-carboxylic acid [2-cyclopentyl-1-(2,3-dimethyl-4-oxo-tetrahydrofuran-3-ylcarbamoyl)-ethyl]-amide

Furan-3-carboxylic acid [2-cyclopentyl-1-(2,3-dimethyl-4-oxo-tetrahydrofuran-3-ylcarbamoyl)-ethyl]-amide

Thiophene-2-carboxylic acid [2-cyclopentyl-1-(2,3-dimethyl-4-oxo-tetrahydrofuran-3-ylcarbamoyl)-ethyl]-amide

Thiophene-3-carboxylic acid [2-cyclopentyl-1-(2,3-dimethyl-4oxo-tetrahydrofuran-3-ylcarbamoyl)-ethyl]-amide

Morpholine-4-carboxylic acid [2-cyclopentyl-1-(2,3-dimethyl-4-oxo-tetrahydrofuran-3-ylcarbamoyl)-ethyl]-amide

N-[2-Cyclopentyl-1-(2,3-dimethyl-4-oxo-tetrahydrofuran-3-ylcarbamoyl)-ethyl]-benzamide

Naphthalene-1-carboxylic acid [2-cyclopentyl-1-(2,3-dimethyl-4-oxo-tetrahydrofuran-3-ylcarbamoyl)-ethyl]-amide

Naphthalene-2-carboxylic acid [2-cyclopentyl-1-(2,3-dimethyl-4-oxo-tetrahydrofuran-3-ylcarbamoyl)-ethyl]-amide

Benzo[b]thiophene-2-carboxylic acid [2-cyclopentyl-1-(2,3-dimethyl-4-oxo-tetrahydrofuran-3-ylcarbamoyl)-ethyl]-amide

Furan-2-carboxylic acid [2-benzylsulfanyl-1-(2,3-dimethyl-4-oxo-tetrahydrofuran-3-ylcarbamoyl)-ethyl]-amide

Furan-3-carboxylic acid [2-benzylsulfanyl-1-(2,3-dimethyl-4-oxo-tetrahydrofuran-3-ylcarbamoyl)-ethyl]-amide

Thiophene-2-carboxylic acid [2-benzylsulfanyl-1-(2,3-dimethyl-4-oxo-tetrahydrofuran-3-ylcarbamoyl)-ethyl]-amide

Thiophene-3-carboxylic acid [2-benzylsulfanyl-1-(2,3-diethyl-4-oxo-tetrahydrofuran-3-ylcarbamoyl)-ethyl]-amide

Morpholine-4-carboxylic acid [2-benzylsulfanyl-1-(2,3-dimethyl-4-oxo-tetrahydrofuran-3-ylcarbamoyl)-ethyl]-amide

Benzo[b]thiophene-2-carboxylic acid [2-benzylsulfanyl-1-(2,3-dimethyl-4-oxo-tetrahydrofuran-3-ylcarbamoyl)-ethyl]-amide

N-[2-Benzylsulfanyl-1-(2,3-dimethyl-4-oxo-tetrahydrofuran-3-ylcarbamoyl)-ethyl]-benzamide

Naphthalene-1-carboxylic acid [2-benzylsulfanyl-1-(2,3-dimethyl-4-oxo-tetrahydrofuran-3-ylcarbamoyl)-ethyl]-amide

Naphthalene-2-carboxylic acid [2-benzylsulfanyl-1-(2,3-dimethyl-4-oxo-tetrahydrofuran-3-ylcarbamoyl)-ethyl]-amide

Furan-2-carboxylic acid [1-(2,3-dimethyloxo-tetrahydrofuran-3-ylcarbamoyl)-2-phenylmethanesulfonyl-ethyl]-amide

Furan-3-carboxylic acid [1-(2,3dinmethyl-4-oxo-tetrahydrofuran-3-ylcarbamoyl)-2-phenylmethesulfonyl-ethyl]-amide

Thiophene-2-carboxylic acid [1-(2,3-dimethyl-4-oxo-tetrahydrofuran-3-ylcarbamoyl)-2-phenylmethanesulfonyl-ethyl]-amide

Thiophene-3-carboxylic acid [1-(2,3-dimethyl-4-oxo-tetrahydrofuran-3-ylcarbamoyl)-2-phenylmethanesulfonyl-ethyl]-amide

Morpholine-4-carboxylic acid [1-(2,3-dimethyl-4-oxo-tetrahydrofuran-3-ylcarbamoyl)-2-phenylmethanesulfonyl-ethyl]-amide

Benzo[b]thiophene-2-carboxylic acid [t-(2,3-dimethyl-4-oxo-tetrahydrofuran-3-ylcarbamoyl)-2-phenylmethanesulfonyl-ethyl]-amide

N-[1-(2,3-Dimethyl-4-oxo-tetrahydrofuran-3-ylcarbamoyl)-2-phenylmethanesulfonyl-ethyl]-benzamide

Naphthalene-1-carboxylic acid [1-(2,3-dimethyl-4-oxo-tetrahydrofuran-3-ylcarbamoyl)-2-phenylmethanesulfonyl-ethyl]-amide

Naphthalene-2-carboxylic acid [1-(2,3-dimethyl-4-oxo-tetrahydrofuran-3-ylcarbamoyl)-2-phenylmethanesulfonyl-ethyl]-amide

2-Benzyloxy-3-cyclohexyl-N-(2,3-dimethyl-4-oxo-tetrahydrofuran-3-yl)-propionamide

2-(4-tert-Butyl-benzylsulfanyl)-4-methyl-pentanoic acid (2,3-dimethyl-4-oxo-tetrahydrofuran-3-yl)-amide

2-(4-tert-Butyl-phenylmethanesulfonyl)-4-methyl-pentanoic acid (2,3-dimethyl-4-oxo-tetrahydrofuran-3-yl)-amide

2tert-Butyl-benzylsulfanyl)-N-(2,3-dimethyl-4-oxo-tetrahydrofuran-3-yl)-3-(4 hydroxyphenyl)-propionamide

2-(4-tert-Butyl-phenylmethanesulfonyl)-N-(2,3-dimethyl-4-oxo-tetrahydrofuran-3-yl)-3-(4-hydroxyphenyl)-propionamide

2-Cyclohexylmethyl-N-(2,3-dimethyl-4-oxo-tetrahydrofuran-3-yl)-4-morpholin-4-yl-4-oxo-butyramide

4,4-Dimethyl-2-(2-morpholin-4-yl-2-oxo-ethyl)-pentanoic acid (2,3-dimethyl-4-oxo-tetrahydrofuran-3-yl)-amide

2-Biphenyl-3-ylmethyl-pentanoic acid (2,3-dimethyl-4-oxo-tetrahydro-furan-3-yl)-amide

4-(3,4-Dihydro-1H-isoquinolin-2-yl)-N-(2,3-dimethyl-4-oxo-tetrahydrofuran-3-yl)-2-(4-hydroxy-benzyl)-4-oxo-butyramide

N-(2,3-Dimethyl-4-oxo-tetrahydro-furan-3-yl)-2-(4-hydroxybenzyl)-4oxo-4-(3-phenyl-pyrrol-1-yl)-butyramide

N-(2,3-Dimethyl-4-oxo-tetrahydrofuran-3-yl)-2-(4-hydroxy-benzyl)-4-oxo-4-(3-phenyl-pyrrolidin-1-yl)-butyramide

4-Methyl-2-[2-oxo-23-phenyl-pyrrol-1-yl)-ethyl]-pentanoic acid (2,3-methyl-4-oxo-tetrahydrofuran-3-yl)-amide

4-Methyl-2-[2-oxo-2-(3-phenyl-pyrrolidin-1-yl)-ethyl]-pentanoic acid (2,3-dimethyl-4-oxo-tetrahydrofuran-3-yl)-amide

2-[2-(1,3-Dihydro-isoindol-2-yl)-2-oxo-ethyl]-4-methyl-pentanoic acid (2,3-dimethyl-4-oxo-tetrahydrofuran-3-yl)-amide

(1,3-Dihydro-isoindol-2-yl)-N-(2,3-dimethyl-4-oxo-tetrahydrofuran-3-yl)-2-(4-hydroxy-benzyl)-4-oxo-butyramide

2-[2-(3,4-Dihydro-1H-isoquinolin-2-yl)-2-oxo-ethyl]4-methyl-pentanoic acid (2,3-dimethyl-4oxo-tetrahydrofuran-3-yl)-amide

Additional compounds of the invention include, but are not limited to, the following examples that are the (2S, 3R) isomer of general formula (I) where Z=‘O’ and R¹=‘H’, and also include the equivalent analogues included in the fall definition of Z and R′, R² and R³

4-tert-Butyl-N-[1-(2,3-dimethyl-4-oxo-tetrahydrofuran-3-ylcarbamoyl)-2-(4 hydroxyphenyl)-ethyl]-benzamide

N-[1-(2,3-Dimethyl-4oxo-tetrahydrofuran-3-ylcarbamoyl)-2-(4-hydroxyphenyl)-ethyl]4trifluoromethoxybenzamide

4-Dimethylamino-N-[1-(2,3-methyl-4oxo-tetrahydrofuran-3-ylcarbamoyl)-2-(4-hydroxyphenyl)ethyl]-benzamide

N-[1-(2,3-Dimethyl-4oxo-tetrahydrofuran-3-ylcarbamoyl)-2-(4-hydroxyphenyl)-ethyl]-4-isopropylbenzamide

4-Difluoromethoxy-N-[1-(2,3-dimethyl-4-oxo-tetrahydrofuran-3-ylcarbamoyl)-2-(4 hydroxyphenyl)-ethyl]-benzamide

N-[1-(2,3-Dimethyl-4-oxo-tetrahydrofuran-3-ylcarbamoyl)-2-(4-hydroxyphenyl)-ethyl]4-trifluoromethylbenzamide

Benzo[b]thiophene-2-carboxylic acid [1-(2,3-dimethyl-4-oxo-tetrahydrofuran-3-ylcarbamoyl)-2-(4-hydroxyphenyl)-ethyl]-amide

Benzo[b]thiophene-3-carboxylic acid [1-(2,3-dimethyl-4-oxo-tetrahydrofuran-3-ylcarbamoyl)-2-(4-hydroxyphenyl)-ethyl]-amide

Benzothiazole-5-carboxylic acid [1-(2,3-dimethyl-4-oxo-tetrahydrofuran-3-ylcarbamoyl)-2-(4-hydroxyphenyl)-ethyl]-amide

N-[1-(2,3-Dimethyl-4-oxo-tetrahydrofuran-3-ylcarbamoyl)-2-(4-hydroxyphenyl)-ethyl]4thiophen-2-ylbenzamide

5-Phenylthiophene-2-carboxylic acid [1-(2,3-dimethyl-4-oxo-tetrahydrofuran-3-ylcarbamoyl)-2-(4-hydroxyphenyl)-ethyl]-amide

4-Phenylthiophene-2-carboxylic acid [1-(2,3-dimethyl-4-oxo-tetrahydrofuran-3-ylcarbamoyl)-2-(4-hydroxyphenyl)-ethyl]-amide

4-tert-Butyl-N-[1-(2,3-dimethyl-4-oxo-tetrahydrofuran-3-ylcarbamoyl)-3-methyl-butyl]-benzamide

N-[1-(2,3-Dimethyl-4-oxo-tetrahydrofuran-3-ylcarbamoyl)-3-methyl-butyl]-4-trifluoromethoxybenzamide

4-Dimethylamino-N-[1-(2,3dimethyloxo-tetrahydrofuran-3-ylcarbamoyl)-3-methyl-butyl]-benzamide

4-Isopropyl-N-[1-(2,3-dimethyl-4oxo-tetrahydrofuran-3-ylcarbamoyl)-3-methyl-butyl]-benzamide

4-Difluoromethoxy-N-[1-(2,3-dimethyl-4oxo-tetrahydrofuran-3-ylcarbamoyl)-3-methyl-butyl]-benzamide

N-[1-(2,3-Dimethyl-4-oxo-tetrahydrofuran-3-ylcarbamoyl)-3-methyl-butyl]4 trifluoromethylbenzamide

Benzo[b]thiophene-2-carboxylic acid [1-(2,3-dimethyl-4-oxo-tetrahydrofuran-3-ylcarbamoyl)-3-methyl-butyl]-amide

Benzo[b]thiophene-3-carboxylic acid [1-(2,3-dimethyl-4oxo-tetrahydrofuran-3-ylcarbamoyl)-3-methyl-butyl]-amide

Benzothiazole-5-carboxylic acid [1-(2,3-dimethyl-4-oxo-tetrahydrofuran-3-ylcarbamoyl)-3-methyl-butyl]-amide

N-[1-(2,3-Dimethyl-4-oxo-tetrahydrofuran-3-ylcarbamoyl)-3-methyl-butyl]4-thiophen-2-ylbenzamide

5-Phenylthiophene-2-carboxylic acid [[1-(2,3-dimethyl-4-oxo-tetrahydrofuran-3-ylcarbamoyl)-3-methyl-butyl]-amide

4-Phenylthiophene-2-carboxylic acid [1-(2,3-methyl-4oxo-tetrahydrofuran-3-ylcarbamoyl)-3-methyl-butyl]-amide

Further additional compounds of the invention include, but are not limited to, the following examples that are the (2S, 3R) isomer of general formula (I) where Z=‘O’ and R¹=‘H’, and also include the equivalent analogues included in the fulll definition of Z and R′, R² and R³

Furan-3-carboxylic acid [1-(2,3-dimethyl-4-oxo-tetrahydrofuran-3-ylcarbamoyl)-2-(4-hydroxyphenyl)-ethyl]-amide

Thiophene-3-carboxylic acid [1-(2,3-dimethyloxo-tetrahydrofuran-3-ylcarbamoyl)-2-(4hydroxyphenyl)-ethyl]-amide

Furan-3-carboxylic acid [1-(2,3-dimethyl-4-oxo-tetrahydrofuran-3-ylcarbamoyl)-3-methyl-butyl]-amide

Thiophene-3-carboxylic acid [1-(2,3-dimethyl-4-oxo-tetrahydrofuran-3-ylcarbamoyl)-3-methyl-butyl]-amide

Furan-3-carboxylic acid [2-cyclohexyl-1-(2,3-dimethyl-4-oxo-tetrahydrofuran-3-ylcarbamoyl)ethyl]-amide

Thiophene-3-carboxylic acid [2-cyclohexyl-1-(2,3-dimethyl-4-oxo-tetrahydrofuran-3-ylcarbamoyl)-ethyl]-amide

Furan-3-carboxylic acid 1-(2,3-dimethyloxo-tetrahydrofuran-3-ylcarbamoyl)-3,3-dimethyl-butyl]-amide

Thiophene-3-carboxylic acid 1-(2,3-dimethyl-4oxo-tetrahydrofuran-3-ylcarbamoyl)-3,3dimethyl-butyl]-amide

Furan-3-carboxylic acid [2-cyclopentyl-1-(2,3-dimethyl-4-oxo-tetrahydrofuran-3-ylcarbamoyl)-ethyl]-amide

Thiophene-3-carboxylic acid [2-cyclopentyl-1-(2,3-dimethyl-4-oxo-tetrahydrofuran-3-ylcarbamoyl)-ethyl]-amide

Furan-3-carboxylic acid [2-benzylsulfanyl-1-(2,3-dimethyl-4oxo-tetrahydrofuran-3-ylcarbamoyl)-ethyl]-amide

Thiophene-3-carboxylic acid [2-benzylsulfanyl-1-(2,3-dimethyl-4-oxo-tetrahydrofuran-3-ylcarbamoyl)-ethyl]-amide

Furan-3-carboxylic acid [1-(2,3-dimethyl-4oxo-tetrahydrofuran-3-ylcarbamoyl)-2-phenylmethanesulfonyl-ethyl]-amide

Thiophene-3-carboxylic acid [1-(2,3-dimethyl-4-oxo-tetrahydrofuran-3-ylcarbamoyl)-2-phenylmethanesulfonyl-ethyl]-amide

Considering all of the above examples, it is also intended to include the oxidised analogues of capping groups that contain a readily oxidised nitrogen to give the N-oxide or a readily oxidised sulphur to give the sulphone. The following structures are illustrative examples;

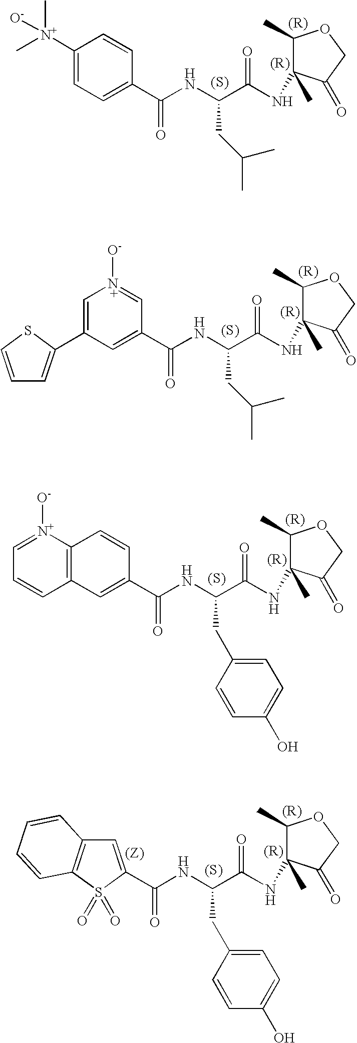

To those skilled in the practices of organic chemistry, compounds of general formula (I) may be readily synthesised by a number of chemical strategies, performed either in solution or on the solid phase (see Atherton, E. and Sheppard, R. C. In ‘Solid Phase Peptide Synthesis: A Practical Approach’, Oxford University Press, Oxford, U.K. 1989, for a general review of solid phase synthesis principles). The solid phase strategy is attractive in being able to generate many thousands of analogues, typically on a 5-100 mg scale, through established parallel synthesis methodologies (e.g. see (a) Bastos, M Maeji, N. J.; Abeles, R. H. Proc. Natl. Acad. Sci. USA, 92, 6738-6742, 1995).

Therefore, one strategy for the synthesis of compounds of general formula (I) comprises:—

(a) Preparation of an appropriately functionalised and protected (2-alkyl-3-alkyl-4-oxo-tetrahydrofuran-3-yl)amide or (2-alkyl-3-alkyl-4-oxo-tetrahydrothiophen-3-yl)amide or (1-alkyl-2-alkyl-5-oxocyclopentyl)amide ketone building block in solution.

(b) Attachment of the building block (a) to the solid phase through a linker that is stable to the conditions of synthesis, but readily labile to cleavage at the end of a synthesis (see James, I. W., Tetrahedron, 55(Report N° 489), 4855-4946, 1999, for examples of the ‘linker’ function as applied to solid phase synthesis).

(c) Solid phase organic chemistry (see Brown, R. D. J. Chem. Soc., Perkin Trans. 1, 12, 3293-3320, 1998), to construct the remainder of the molecule.

(d) Compound cleavage from the solid phase into solution.

(e) Cleavage work-up and compound analysis.

The first stage in a synthesis of compounds of general formula (I) is the preparation in solution of a functionalised and protected building block. A typical scheme towards the (2-alkyl-3-alkyl-4-oxo-tetrahydrofuran-3-yl)amide (7) is detailed in Scheme 1.

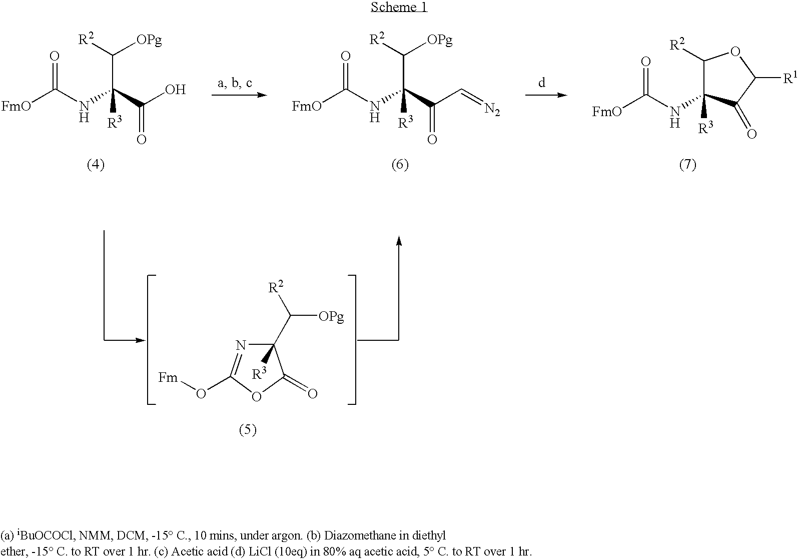

FmOC(O) denotes the well known amine protecting group 9-fluorenyl methoxycarbonyl (Fmoc, see Atherton, E. and Sheppard, R. C., 1989) and ‘Pg’ denotes either a free hydroxyl or an hydroxylprotecting group such as tert-butyl ether. In the illustrated case, condensation with diazomethane provides R¹=H.

Considering step (a), synthesis may commence from suitably protected β-hydroxy-α-alkyl-α-aminoacid (4). β-hydroxy-α-aminoacids are accessible through a variety of literature methods e.g. (a) Adams, Z. M., Jackson, R F. W., Palmer, N. J., Rami, H. K., Wythes, M. J., J. Chem. Soc., Perkin Trans 1,937-947, 1999, (b) Hubschwerlen, C., et al, J. Med. Chem, 41, 3972-3975, 1998, (c) Luzzio, F. A., et al, Tet. Lett, 4a, 7151-7155, 2000, (d) Morgan, A. J. et al, Org. Lett. 1(12), 1949-1952, 1999. (e) Zhang, H., xia, P., Zhou, W., Tetrahedron: Asymmetry, 1, 3439-3447, 2000, (f) Blaskovich, M. A., et al, J. Org. Chem, 6, 3631-3646, 1998, (g) Seebach, D. et al, Helv. Chim. Acta., 7, 1194, 1987. Also, α-alkyl-α-aminoacids are readily available through a number of literature methods e.g. Cativiela, C., Diaz-de-Villegas, M. D. Tetrahedron: Asymmetry, 9. 3517-3599, 1998.

In the simplest case where R² and R³ are a methyl substituent, the β-hydroxy-α-alkyl-α-aminoacid (4) is the known (2R, 3SR)-α-methylthreonine (the 35 isomer corresponds to D-Thr and the 3R isomer corresponds to D-alloThr). Activation of the suitably protected β-hydroxy-α-alkyl-α-aminoacid (4) via isobutyl chloroformate mixed anhydride, followed by condensation with diazomethane, yields the diazomethylketone intermediate (6). Following the reaction conditions detailed in Scheme 1, formation of the diazoketone is clearly observed. However, an overall improvement in isolated yield of diazoketone (6) is obtained by pre-forming the acyl fluoride of (4), which has a lesser propensity to form the poorly active (1′R or S, 4R) 4-(1′-alkoxyethyl)-2-(9H-fluoren-9-ylmethoxy)-4-methyl-4H-oxazol-5-one (5) and also by leaving the reaction with ethereal diazomethane for 48 hr. Treatment of diazomethylketone intermediate (6) with lithium chloride in aqueous acetic acid provides the protected (2-alkyl-3-alkyl-4-oxo-tetrahydrofuran-3-yl)amide (7). Introduction of simple R¹ substituents may be achieved by condensation of activated (4) with alternatives to diazomethane such as diazoethane (R¹=CH₃), or 1-phenyloxydiazoethane (R¹=CH₂OPh).

The protected building blocks (synthesis exemplified by the (2-alkyl-3-alkyl-4-oxo-tetrahydrofuran-3-yl)amide (7)) detailed in Scheme 1 may be utilised in a solid phase synthesis of inhibitor molecules (steps (b) to (e)). Step (b), the solid phase linkage of an aldehyde or ketone, has previously been described by a variety of methods (e.g. see (a) James, I. W., 1999, (b) Lee, A., Huang, L., Ellman, J. A., J. Am. Chem. Soc, 121(43), 9907-9914, 1999, (c) Murphy, A. M., et al, J. Am. Chem. Soc, 14, 3156-3157, 1992). A suitable method amenable to the reversible linkage of an alkyl ketone functionality such as (7) is through a combination of the previously described chemistries. The semicarbazide, 4-[[(hydrazinocarbonyl)amino]methyl]cyclohexane carboxylic acid.trifluoroacetate (8) (Murphy, A. M., et al, J. Am. Chem. Soc, 114, 3156-3157, 1992), may be utilised as illustrated in Scheme 2, exemplified by linkage of the (2-alkyl-3-alkyl-4-oxo-tetrahydrofuran-3-yl)amide (7).

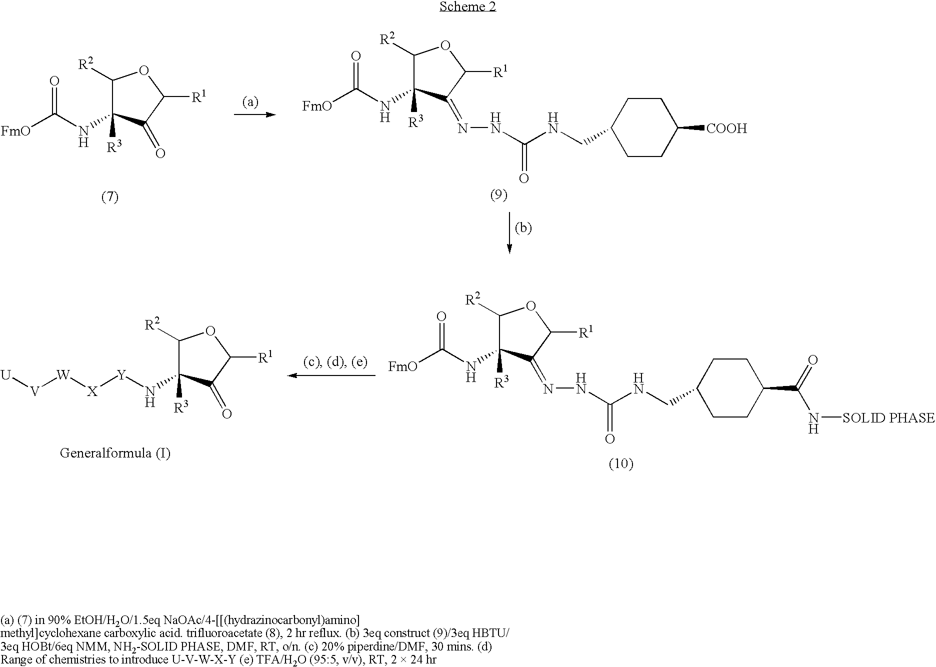

Construct (9) is prepared through reaction of the linker molecule (8) and the (2-alkyl-3-alkyl-4-oxo-tetrahydrofuran-3-yl)amide (7) by reflux in aqueous ethanol/sodium acetate. Although formation of construct (9) is observed at 2 hr reaction, optimal formation of construct (9) occurs at 72 hr reaction. Standard solid phase techniques (e.g. see Atherton, E. and Sheppard, R. C., 1989) are used to anchor the construct to an amino-functionalised solid phase through the free carboxylic acid functionality of (9), providing the loaded construct (10). Acid mediated cleavage of the fully constructed compounds is optimal at a repeated 24 hr reaction.

Loaded construct (10) may be reacted with a wide range of carboxylic acids available commercially or in the literature, to introduce the left-hand portion ‘U-V-W-X-Y’ in general formula (I). In the simplest example, the entire left hand portion of an inhibitor of general formula (I) comprises a capped aminoacid (Scheme 3), providing for example analogues of general formula (I) where R⁴=‘H’, (X)ₒ=‘−’, (W)ₙ=‘NH’, R⁸=‘H’, n=1, (V)ₘ=‘CO’, m=1 and U=aryl

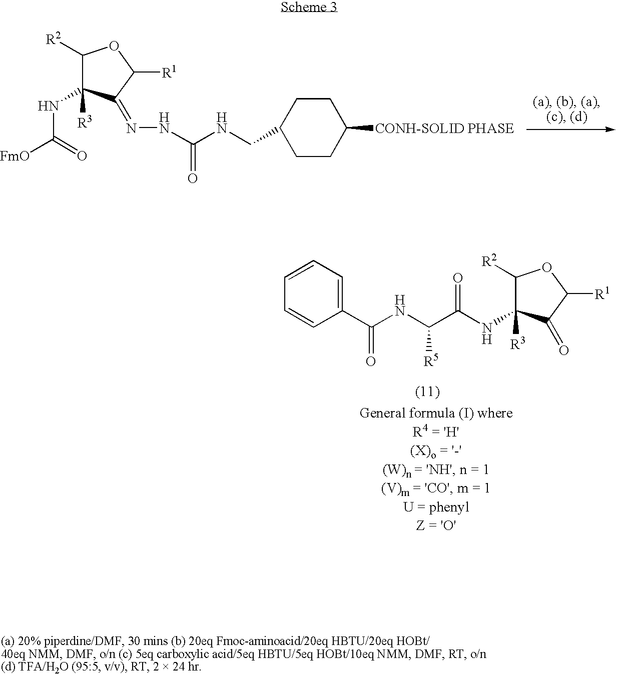

Alternatively, carboxylic acids can be prepared in solution by traditional organic chemistry methods and coupled to construct (10) on the solid phase (Schemes 4-8). For example (Scheme 4), treatment in solution of an amino acid, exemplified by (12) with sodium nitrite/H₂SO₄, provides the α-hydroxyacid, exemplified by (13) (Degerbeck, F. et al, J. Chem. Soc, Perkin Trans. 1, 11-14, 1993). Treatment of α-hydroxyacid, (13) with sodium hydride in a dimethylformamide/dichloromethane mixture followed by addition of benzyl bromide, provides 2RS-benzyloxy-3-cyclohexylpropionic acid (14). Coupling of (14) to the solid phase construct (10) followed by cleavage, provides (15), an example of general formula (I) where R⁴=‘H’, (P)o=‘-’, (W)ₙ=‘O’, n=1, (V)ₘ=‘CH₂’, i.e. R⁹, R¹⁰=‘H’, m=1 and U=phenyl. To those skilled in the practices of organic synthesis, a wide variety of aminoacids such as (12) may be converted to the corresponding α-hydroxyacid such as (13) following the general conditions detailed. Additionally, benzylbromide may be replaced by any reasonable Ar—CR⁹R¹⁰-halide, providing many variations of carboxylic acid (14) following the general conditions detailed. In certain instances, it may be advantageous to temporarily protect the carboxylic acid as the methyl ester (for example compound (20), Scheme 6) prior to reaction with the allylhalide. The ester intermediate is then simply hydrolysed to acid (14). Analogues of (15), exploring a wide range of (V)ₘ and U in general formula (I) may be prepared through the general conditions detailed in Scheme 4. Since the final synthetic step involves a trifluoroacetic acid (TFA) mediated cleavage of the solid phase bound compound, analogues where the substituted ether is labile to TFA may be prepared in solution by an alternative route (see Scheme 11).

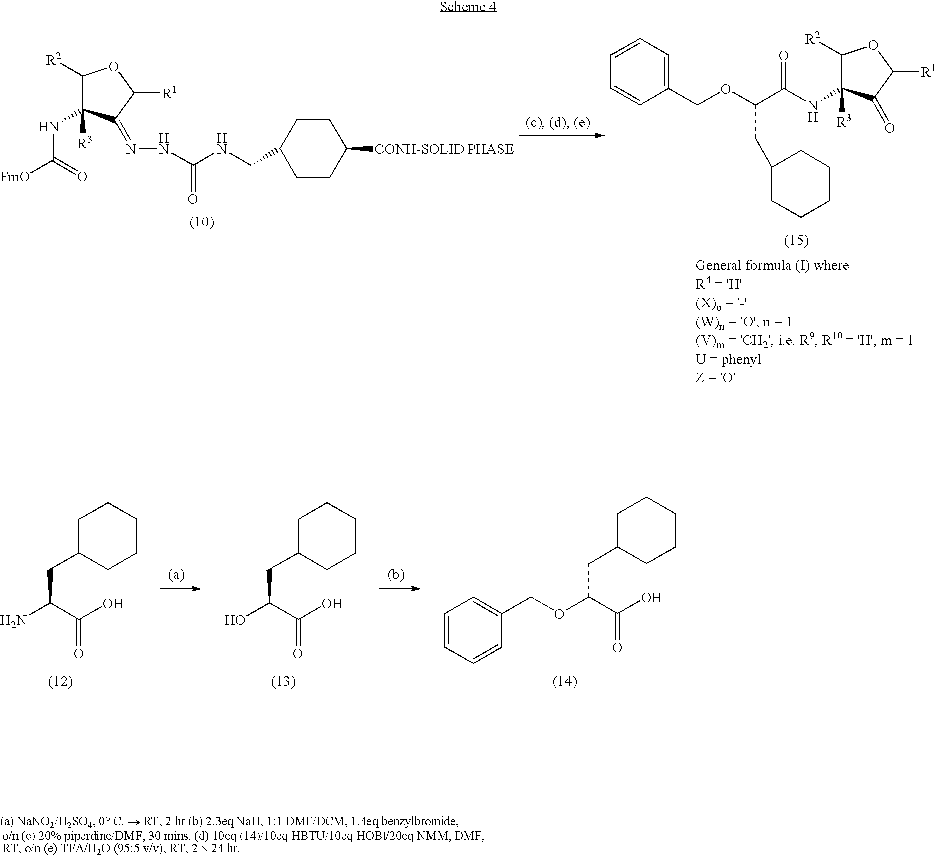

Alternatively, coupling of construct (10) (following removal of Fmoc) with the α-hydroxyacid (13), provides a versatile solid phase bound intermediate ‘Y’ substituent in general formula (I) that may be reacted with many reagents. For example, the α-hydroxyl can be reacted under Mitsunobu conditions (Hughes, D. L. Org. React.(N.Y.), 42, 335-656, 1992) to give ethers (i.e. X=‘−’, W=‘O’, in general formula (I)) (see Grabowska, U. et al, J. Comb. Chem., 2(5), 475-490, 2000, for an example of Mitsunobu reaction on the solid phase). Alternatively, the α-hydroxyl can be reacted with a carbamoyl chloride to give a carbamate (i.e. X=‘−’, W=‘O’, V=‘NHC(O)’, in general formula (I)).

Alternatively, (Scheme 5), treatment in solution of an amino acid, exemplified by (12) with sodium nitrite/H₂SO₄/potassium bromide provides the α-bromoacid, exemplified by (16) (Souers, A. J. et al, Synthesis, 4. 583-585, 1999) with retention of configuration. Treatment of α-bromoacid (16) with an alkylthiol exemplified by 4-tert-butylphenylmethanethiol (17) in dimethylformamide/triethylamine, provides 2S-(4-tert-butylbenzylsulfanyl)-4-methylpropionic acid (18), with inversion of configuration. Coupling of (18) to the solid phase construct (10) followed by cleavage, provides (19), an example of general formula (I) where R4=‘H’, (X)ₒ=‘−’, (W)ₙ=‘S’, n=1, (V)ₘ=‘CH₂’, i.e. R⁹, R¹⁰=‘H’, m=1 and U=4-tert-butylphenyl. To those skilled in the practices of organic synthesis, a wide variety of aminoacids such as (12) may be converted to the corresponding α-bromoacid such as (16) following the general conditions detailed. Additionally, starting with the S-isomer of (12) gives the S-bromoacid analogue of (16) and R-thioether analogue of (18). Additionally, (4-tert-butylphenyl)methanethiol (17) may be replaced by any reasonable Ar—CR⁹R¹⁰—SH, providing many variations of carboxylic acid (18) following the general conditions detailed. Thus analogues of (19) exploring a wide range of (V)ₘ and U in general formula (I) may be prepared through the general conditions detailed in Scheme 5.

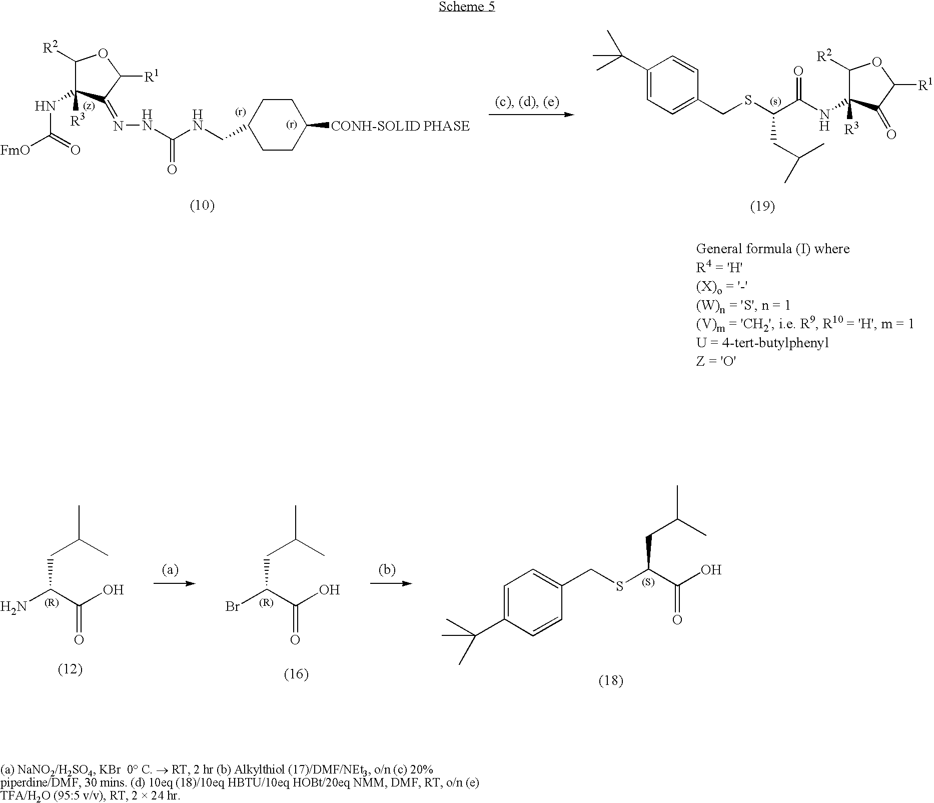

Alternatively, coupling of construct (10) (following removal of Fmoc) with an α-bromoacid e.g. (16), provides a versatile intermediate ‘Y’ substituent in general formula (I) that may be reacted with many reagents. For example, the α-bromide can be displaced with nucleophiles e.g. alcohols, thiols, carbanions etc, to give ethers (i.e. X=‘−’, W=‘O’, in general formula (I)), thioethers (ie. X=‘−’, W=‘S’, in general formula (I)). The thioethers may optionally be oxidised to the sulphone (see Scheme 9, i.e. X=‘−’, W=‘SO₂’, in general formula (I)) (see Grabowska, U. et al, J. Comb. Chem., 2(5), 475-490, 2000, for an example of bromide displacement and thioether oxidation on the solid phase).

Alternatively, (Scheme 6), treatment of an α-hydroxyacid, exemplified by (13) with trimethylsilylchloride and methanol provides the methyl ester (20). Activation of the free hydroxyl to the chloroformate with phosgene in dichloromethane followed by addition of morpholine, then hydrolysis, provides morpholine-4-carboxylic acid-1S-carboxy-2-cyclohexyl ethyl ester (21). Coupling of (21) to the solid phase construct (10) followed by cleavage, provides (22), an example of general formula (I) where R⁴=‘H’, (X)ₒ=‘−’, (W)ₙ=‘O’, n=1, (V)ₘ=‘CO’ and U=morpholino. To those skilled in the practices of organic synthesis, a wide variety of α-hydroxyacid esters such as (20) could be converted to the activated chloroformate following the general conditions detailed. Additionally, morpholine may be replaced by any reasonable amine, providing many variations of carboxylic acid (21) following the general conditions detailed. Thus analogues of (22) exploring a wide range of (V)ₘ and U in general formula (I) may be prepared through the general conditions detailed in Scheme 6.

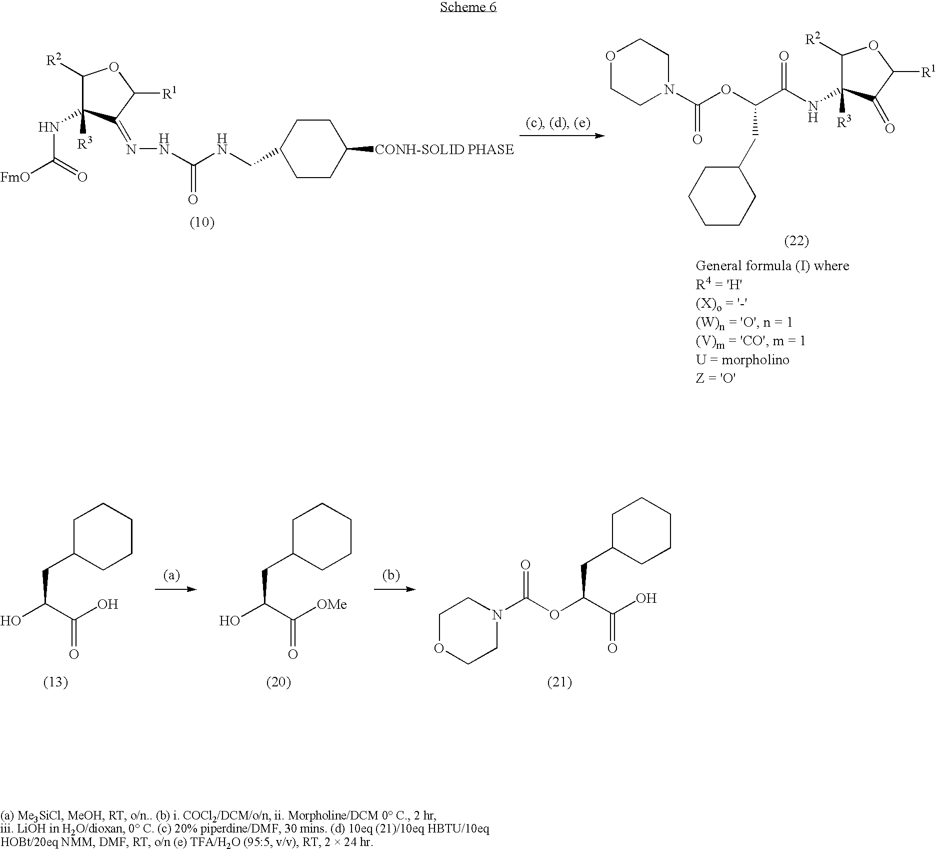

Alternatively, (Scheme 7), a wide range of alkylsuccinate esters exemplified by 2R-cyclohexylmethylsuccinic acid 1-methyl ester (23) are commercially available or readily prepared by known methods (see (a) Azam et al, J. Chem. Soc. Perkin Trans. 1, 621-, 1996; (b) Evans et al, J. Chem. Soc. Perkin Trans. 1, 103, 2127, 1981; (c) Oikawa et al, Tet. Lett, 3, 6169, 1996). Carboxyl activation of alkylsuccinate ester (23) followed by addition of morpholine in dimethylformamide and subsequent ester hydroylsis, provides 2R-cyclohexylmethyl-4-morpholin-4yl-4oxo-butyric acid (24). Coupling of (24) to the solid phase construct (10) followed by cleavage, provides (25), an example of general formula (I) where R⁴=‘H’, (X)ₒ=‘CH₂’ i.e. R⁶, R⁷=‘H’, o=1, (W)ₙ=‘CO’, n=1, (V)ₘ=‘−’ and U=morpholino. To those skilled in the practices of organic synthesis, a wide variety of alkylsuccinate esters such as (23) may be prepared and converted to the corresponding substituted alkylsuccinate acid such as (24) following the general conditions detailed. Additionally, morpholne may be replaced by any reasonable amine, providing many variations of carboxylic acid (24) following the general conditions detailed. Thus analogues of (25) exploring a wide range of (X)ₒ, (V)ₘ and U in general formula (I) may be prepared through the general conditions detailed in Scheme 7.

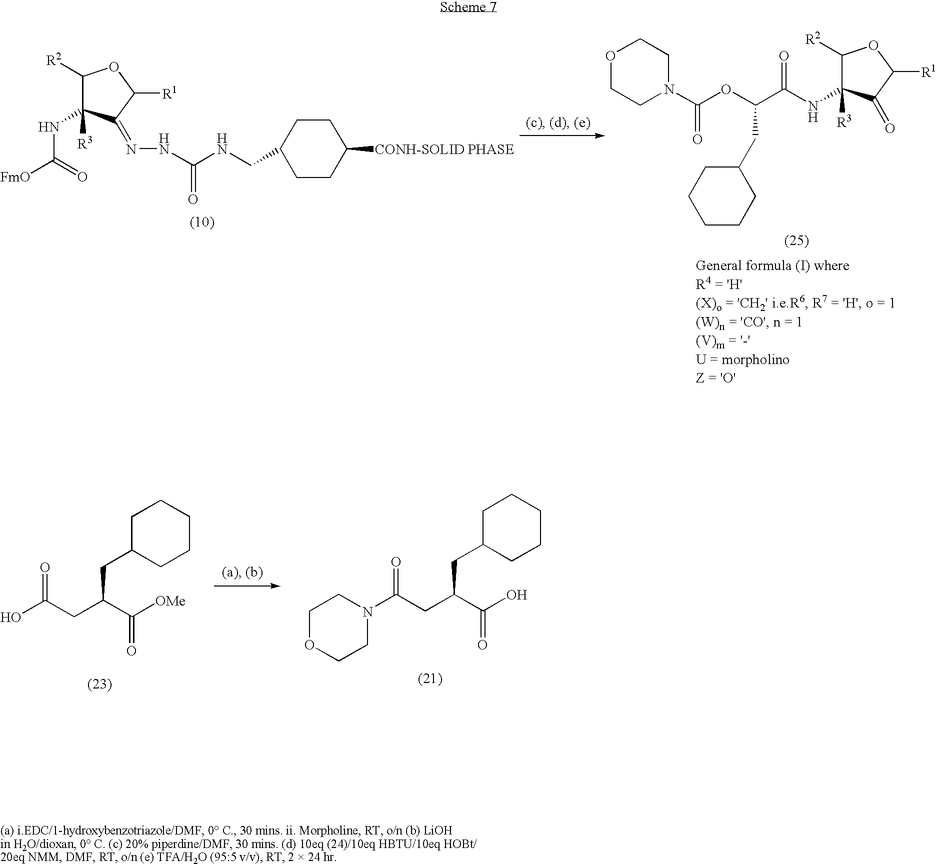

Alternatively, (Scheme 8), a wide range of biarylalkylacetic acids, exemplified by 2RS-biphenyl-3-yl-4-methylpentanoic acid (27) are readily available by known methods (see (a) DesJarlais, R. L. et al, J. Am. Chem. Soc, 120, 9114-9115, 1998; (b) Oballa, R. M. et al, WO 0149288). Coupling of biarylallylacetic acid (27) to the solid phase construct (10) followed by cleavage, provides (28), an example of general formula (I) where R⁴=‘H’, (X)ₒ=‘−’, (W)ₙ=‘−’, (V)ₘ=‘-’ and U=m-biphenyl. To those skilled in the practices of organic synthesis, a wide variety of biarylalkylacetic acids such as (27) may be prepared by alkylation of the α-anion of the free acid analogue of (26), which in turn is prepared by Suzuki coupling of phenylboronic acid and 3-bromophenylacetic acid methyl ester. Phenylboronic acid may be replaced by a wide range of arylboronic acids in the Suzuki coupling, providing many variations of carboxylic acid (27) following the general conditions detailed. Thus analogues of (28) exploring a wide range of group ‘U’ in general formula ([) may be prepared through the general conditions detailed in Scheme 8.

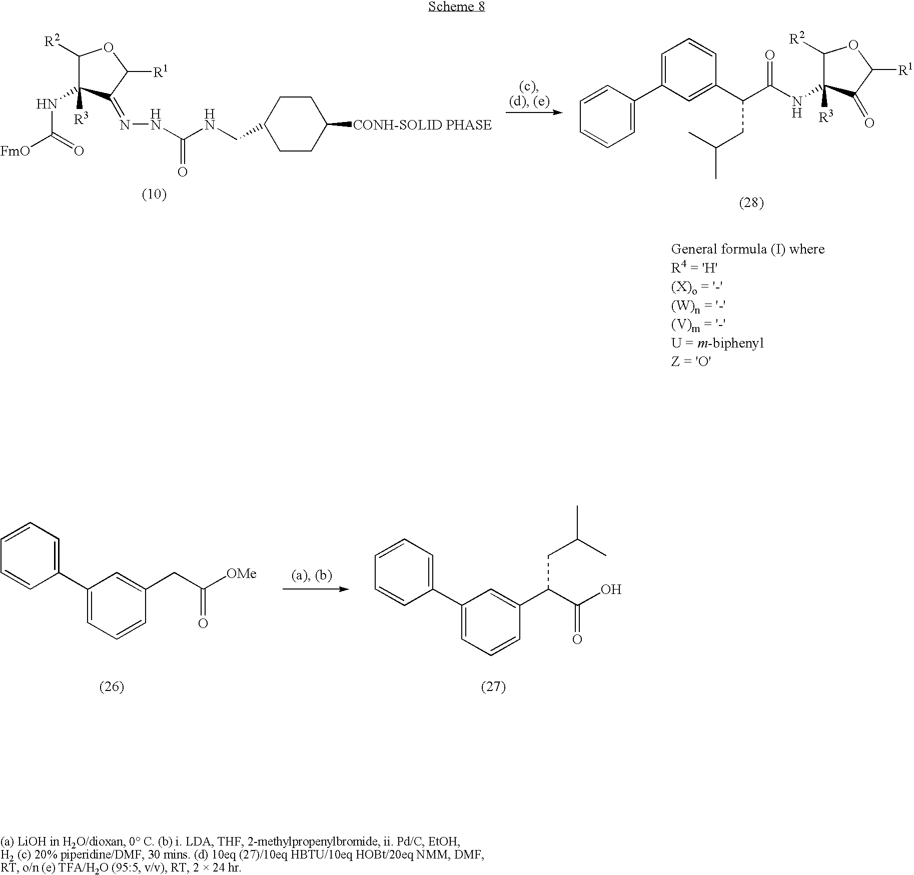

Many other possibilities for solid phase organic chemistry (e.g. see Brown, R. D. J. Chem. Soc., Perkin Trans. 1, 19, 3293-3320, 1998, for a review of recent SPOC publications) can be used to derivatise construct (10) towards compounds of general formula (I). For example, the left-hand portion ‘U-V-W-X-Y’ in general formula (I) can be partially constructed in solution, coupled to construct (10) and further modified on the solid phase. For example (Scheme 9), a simple extension of Scheme 5 is through the oxidation of the intermediate solid phase bound species, with m-chloroperbenzoic acid in dichloromethane prior to cleavage, to give the sulphone analogue (29), an example of general formula (I) where R⁴=‘H’, (X)ₒ=‘−’, (W)ₙ=‘SO₂’, n=1, (V)ₘ=‘CH₂’, i.e. R⁹, R¹⁰=‘H’, m=1 and U=2-furanyl. As described in Scheme 5, many variations of carboxylic acid (18) may be prepared following the general conditions detailed. Thus analogues of (29) exploring a wide range of (V)ₘ and U in general formula (I) may be prepared through the general conditions detailed in Schemes 5 and 9.

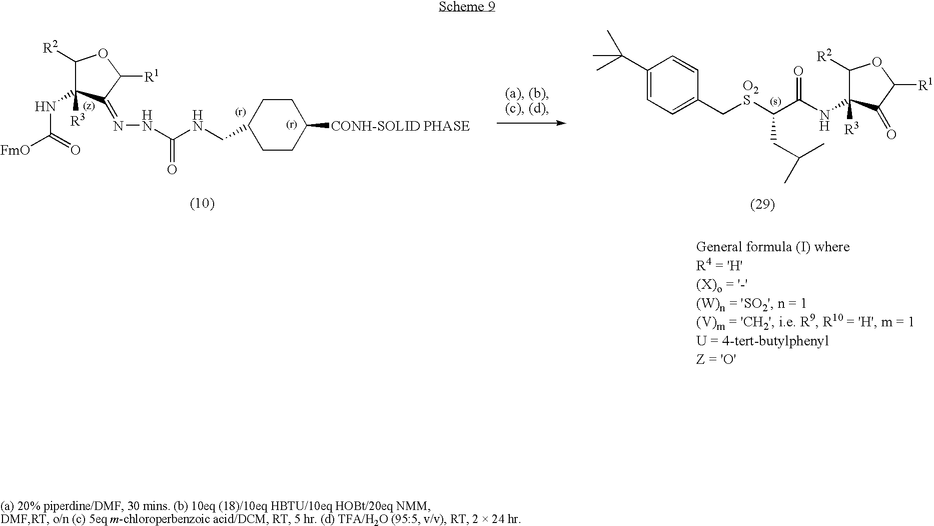

Compounds of general formula (I) are finally released from the solid phase by treatment with trifluoroacetic acid I water, followed by evaporation, lyophylis and standard analytical characterisation.

A second strategy for the synthesis of compounds of general formula (I) comprises:—

(f) Preparation of an appropriately functionalised and protected (2-alkyl-3-alkyl-4-oxo-tetrahydrofuran-3-yl)amide or (2-alkyl-3-alkyl-4oxo-tetrahydrothiophen-3-yl)amide or (1-alkyl-2-alkyl-5-oxocyclopentyl)amide building block in solution. Preferred protecting groups for solution phase chemistry are the Nα-tert-butoxycarbonyl group and the Nα-benzyloxycarbonyl group.

(g) Standard organic chemistry methods for the conversion of building block (f) towards compounds of general formula (I).

In the simplest example, the entire left hand portion of an inhibitor of general formula (I) can be prepared in solution by traditional organic chemistry methods and coupled to building block (t) (see Scheme 10 exemplified by preparation and use of (2-alkyl-3-alkyl-4oxo-tetrahydrofuran-3-yl)carbamic acid tert-butyl ester (32)).

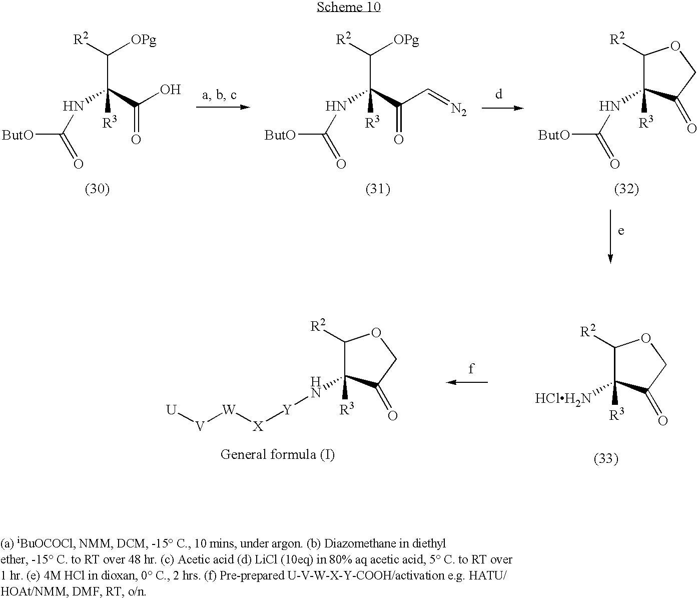

An attractive alternative to the mixed anhydride activation of (30) is through the use of the pre-formed acyl fluoride (skin to that detailed in Scheme 1). The general strategy detailed in Scheme 10 is particularly useful when the compound of general formula (I) contains a substituent that is labile to trifluoroacetic acid, this being the final reagent used in each of the solid phase Schemes 4-9. For example (Scheme 11), treatment in solution of α-hydroxyacid (34) with sodium hydride in a dimethylformamide/dichloromethane mixture followed by addition of 4-tert-butylbenzyl bromide, provides 2RS-(4-tert-butylbenzyloxy)-4methylpentanoic acid (35). Coupling of (35) to hydrochloride salt (33), provides (36), an example of general formula (I) where R⁴=‘H’, (X)ₒ=‘−’, (W)ₙ=‘O’, n=1, (V)ₘ=‘CH₂’, i.e. R⁹, R¹⁰=‘H’, m=1 and U=4-tert-butylphenyl. To those skilled in the practices of organic synthesis, 4-tert-butylbentyl bromide may be replaced by any reasonable Ar-CR⁹R¹⁰-halide, providing many variations of carboxylic acid (35) under the conditions shown. Thus analogues of (36) exploring a wide range of (V)ₘ and U in general formula (I) may be prepared through the conditions detailed in Scheme 11.

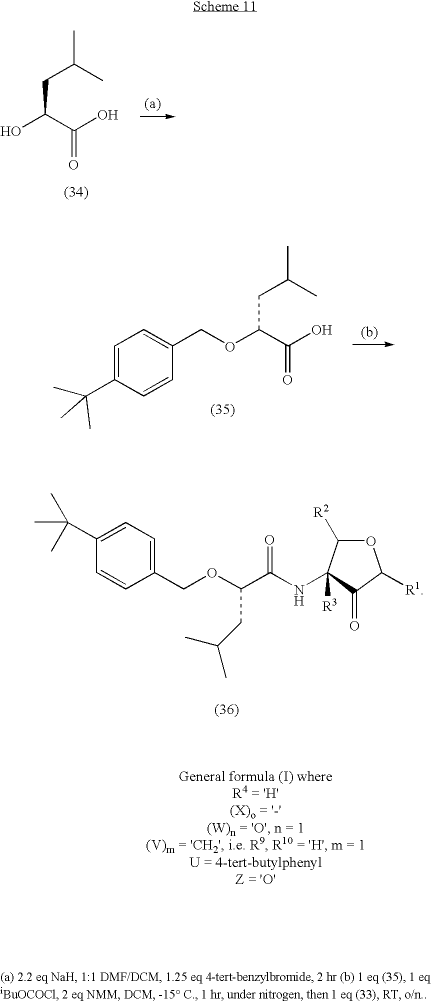

A third strategy for the synthesis of compounds of general formula (I) where the addition of U-V-W-X-Y to the protected (2-alkyl-3-alkyl-4-oxo-tetrahydrofuran-3-yl)amide or (2-alkyl-3-alkyl-4-oxo-tetrahydrothiophen-3-yl)amide or (1-alkyl-2-alkyl-5-oxocyclopentyl)amide building block involves multistep organic reactions comprises:—

(h) Preparation of an appropriately functionalised and protected (2-alkyl-3-alkyl-4-oxo-tetrahydrofuran-3-yl)amide or (2-alkyl-3-alkyl-4-oxo-tetrahydrothiophen-3-yl)amide or (1-alkyl-2-alkyl-5-oxocyclopentyl)amide building block in solution. Preferred protecting groups for solution phase chemistry are the Nα-tert-butoxycarbonyl group and the N-benzyloxycarbonyl group.

(i) Protection of the ketone functionality of the (2-alkyl-3-alkyl-4-oxo-tetrahydrofuran-3-yl)amide or (2-alkyl-3-alkyl-4-oxo-tetrahydrothiophen-3-yl)amide or (1-alkyl-2-alkyl-5-oxocyclopentyl)amide building block e.g. as a dimethylacetal. Alternatively, the ketone may be reduced to the achiral secondary alcohols and re-oxidised as the final synthetic step.

(j) Standard organic chemistry methods for the conversion of building block (i) towards compounds of general formula (I).

Intermediates may be prepared in solution, followed by coupling to building block (i) and further derivatisation towards compounds of general formula (I) (see Scheme 12 exemplified by preparation and use of the (2-alkyl-3-alkyl-4-hydroxytetrahydrofuran-3-yl)carbamic acid tert-butyl ester (37)).

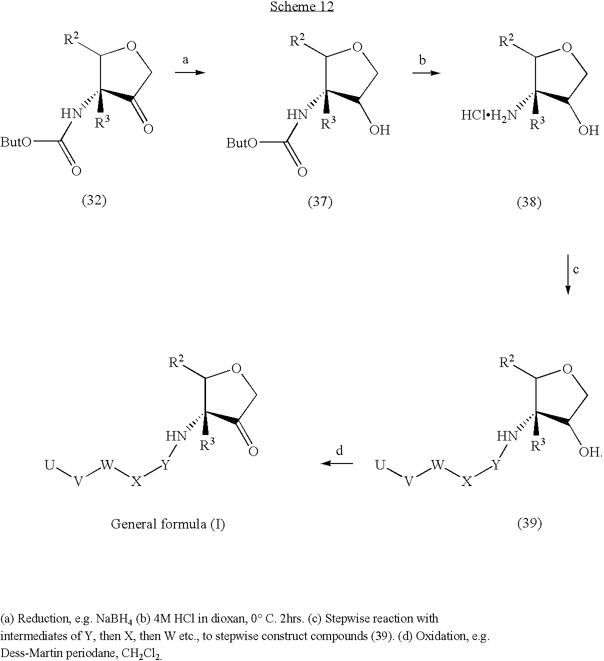

Alternatively, depending upon the types of chemistry used to construct the left hand side U-V-W-X-Y of compounds of general formula (I), the ketone may require protection e.g. as the dimethyl acetal. Such a method is detailed and exemplified in Scheme 13 by the preparation and use of (2-alkyl-3-alkyl-4,4-dimethoxy-tetahydrofuran-3-yl)carbamic acid benzyl ester (41).

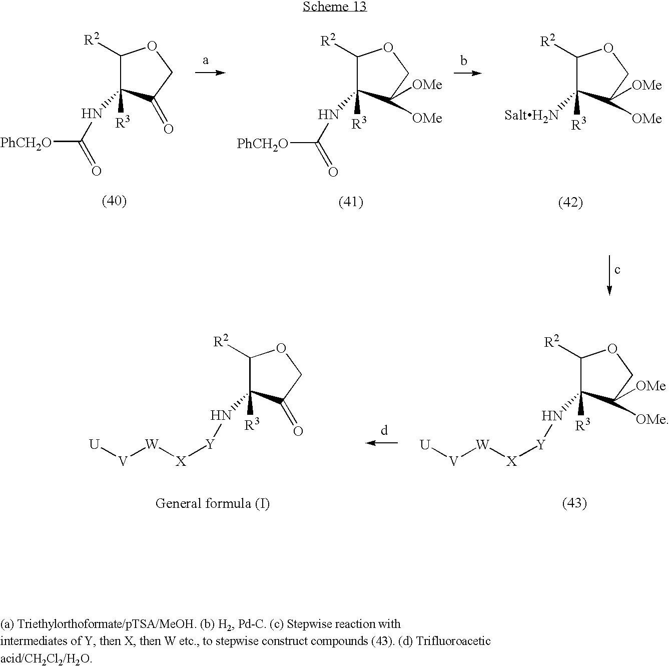

The invention extends to novel intermediates as described above, and to processes for preparing compounds of general formula (I) from each of its immediate precursors. In turn, processes for preparing intermediates from their immediate precursors also form part of the invention.

Compounds of general formula (I) are useful both as laboratory tools and as therapeutic agents. In the laboratory certain compounds of the invention are useful in establishing whether a known or newly discovered cysteine protease contributes a critical or at least significant biochiemical function during the establisbment or progression of a disease state, a process commonly referred to as ‘target validation’.

According to a second aspect of the invention, there is provided a method of validating a known or putative cysteine protease inhibitor as a therapeutic target, the method comprising:

(a) assessing the in vitro binding of a compound as described above to an isolated known or putative cysteine protease, providing a measure of potency; and optionally, one or more of the steps of:

(b) assessing the binding of the compound to closely related homologous proteases of the target and general house-keeping proteases (e.g. trypsin) to provides a measure of selectivity;

(c) monitoring a cell-based functional marker of a particular cysteine protease activity, in the presence of the compound; and

(d) monitoring an animal model-based functional marker of a particular cysteine protease activity in the presence of the compound.

The invention therefore provides a method of validating a known or putative cysteine protease inhibitor as a therapeutic target. Differing approaches and levels of complexity are appropriate to the effective inhibition and ‘validation’ of a particular target. In the first instance, the method comprises assessing the in vitro binding of a compound of general formula (I) to an isolated known or putative cysteine protease, providing a measure of ‘potency’. An additional assessment of the binding of a compound of general formula (I) to closely related homologous proteases of the target and general house-keeping proteases (e.g. trypsin) provides a measure of ‘selectivity’. A second level of complexity may be assessed by monitoring a cell-based functional marker of a particular cysteine protease activity, in the presence of a compound of general formula (I). For example, a ‘human osteoclast resorption assay’ has been utilised as a cell-based secondary in vitro testing system for monitoring the activity of cathepsin K and the biochemical effect of protease inhibitors (e.g. see WO-A-9850533). An ‘MHC-II processing—T-cell activation assay’ has been utilised as a cell-based secondary in vitro testing system for monitoring the activity of cathepsin S and the biochemical effect of protease inhibitors (Shi, G-P., et al, Immunity, 10, 197-206, 1999). When investigating viral or bacterial infections such a marker could simply be a functional assessment of viral (e.g. count of RNA copies) or bacterial loading and assessing the biochemical effect of protease inhibitors. A third level of complexity may be assessed by monitoring an animal model-based functional marker of a particular cysteine protease activity, in the presence of a compound of general formula (I). For example, murine models of Leishmania infection, P. vinckei infection, malaria (inhibition of falcipain) and T. cruzi infection (cruzipain), indicate that inhibition of cysteine proteases that play a key role in pathogen propagation is effective in arresting disease symptoms, ‘validating’ said targets.

The invention therefore extends to the use of a compound of general formula (I) in the validation of a known or putative cysteine protease inhibitor as a therapeutic target.

Compounds of general formula (I) are useful for the in vivo treatment or prevention of diseases in which participation of a cysteine protease is implicated.

According to a third aspect of the invention, there is provided a compound of general formula (I) for use in medicine, especially for preventing or treating diseases in which the disease pathology may be modified by inhibiting a cysteine protease.

According to a fourth aspect of the invention, there is provided the use of a compound of general formula (I) in the preparation of a medicament for preventing or treating diseases in which the disease pathology may be modified by inhibiting a cysteine protease.

Certain cysteine proteases function in the normal physiological process of protein degradation in animals, including humans, e.g. in the degradation of connective tissue. However, elevated levels of these enzymes in the body can result in pathological conditions leading to disease. Thus, cysteine proteases have been implicated in various disease states, including but not limited to, infections by Pneumocystis carinii, Trypsanoma cruzi, Trypsanoma brucei brucei and Crithidia fusiculata; as well as in osteoporosis, autoimmunity, schistosomiasis, malaria, tumour metastasis, metachromatic leukodystrophy, muscular dystrophy, amytrophy, and the like. See WO-A-9404172 and EP-A-0603873 and references cited in both of them. Additionally, a secreted bacterial cysteine protease from S. Aureus called staphylopain has been implicated as a bacterial virulence factor (Potempa, J., et al. J. Biol. Chem, 262(6), 2664-2667, 1998).

The invention is useful in the prevention and/or treatment of each of the disease states mentioned or implied above. The present invention also is useful in a methods of treatment or prevention of diseases caused by pathological levels of cysteine proteases, particularly cysteine proteases of the papain superfamily, which methods comprise administering to an animal, particularly a mammal, most particularly a human, in need thereof a compound of the present invention. The present invention particularly provides methods for treating diseases in which cysteine proteases are implicated, including infections by Pneumocystis carini, Trypsanoma cruzi, Trypsanoma brucei, Leishmania mexicana, Clostridium histolyticum, Staphylococcus aureus, foot-and-mouth disease virus and Crithidia fusiculata; as well as in osteoporosis, autoimmunity, schistosomiasis, malaria, tumour metastasis, metachromatic leukodystrophy, muscular dystrophy and amytrophy.

Inhibitors of cruzipain, particularly cruzipain-specific compounds, are useful for the treatment of Chagas' disease.

In accordance with this invention, an effective amount of a compound of general formula (I) may be administered to inhibit the protease implicated with a particular condition or disease. Of course, this dosage amount will further be modified according to the type of administration of the compound. For example, to achieve an “effective amount” for acute therapy, parenteral administration of a compound of general formula (I) is preferred An intravenous infusion of the compound in 5% dextrose in water or normal saline, or a similar formulation with suitable excipients, is most effective, although an intramuscular bolus injection is also useful. Typically, the parenteaal dose will be about 0.01 to about 100 mg/kg; preferably between 0.1 and 20 mg/kg, in a manner to maintain the concentration of drug in the plasma at a concentration effective to inhibit a cysteine protease. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect. Prodrugs of compounds of the present invention may be prepared by any suitable method. For those compounds in which the prodrug moiety is a ketone functionality, specifically ketals and/or hemiacetals, the conversion may be effected in accordance with conventional methods.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

According to a fifth aspect of the invention, there is provided a pharmaceutical or veterinary composition comprising one or more compounds of general formula (I) and a pharmaceutically or veterinarily acceptable carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for rectal, nasal, topical (including buccal and sublingual), vaginal or parenteral (including subcutaneous, intramuscular, intravenous and intradermal) administration, but preferably the formulation is an orally administered formulation. The formulations may conveniently be presented in unit dosage form, e.g. tablets and sustained release capsules, and may be prepared by any methods well known in the art of pharmacy.

Such methods include the step of bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

Formulations for oral administration in the present invention may be presented as: discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

According to a sixth aspect of the invention, there is provided a process for the preparation of a pharmaceutical or veterinary composition as described above, the process comprising bringing the active compound(s) into association with the carrier, for example by admixture.

Preferred features for each aspect of the invention are as for each other aspect mutatis mutandis.

The invention will now be illustrated with the following examples:

Solution Phase Chemistry—General Methods

All solvents were purchased from ROMIL Ltd (Waterbeach, Cambridge, UK) at SpS or Hi-Dry grade unless otherwise stated. General peptide synthesis reagents were obtained from Chem-Impex Intl. Inc. (Wood Dale IL 60191. USA). Thin layer chromatography (TLC) was performed on prcoated plates (Merck aluminium sheets silica 60 F254, part no. 5554). Visualisation of compounds was achieved under ultraviolet light (254 nm) or by using an appropriate staining reagent. Flash column purification was performed on silica gel 60 (Merck 9385). All analytical HPLC were obtained on Phenomenex Jupiter C₄, 51, 300A, 250×4.6 mm, using mixtures of solvent A=0.1% aq trifluoroacetic acid (TFA) and solvent B=90% acetonitrile/10% solvent A on automated Agilent systems with 215 and/or 254 nm UV detection. Unless otherwise stated a gradient of 10-90% B in A over 25 minutes at 1.5 mL/min was performed for full analytical HPLC analysis. HPLC-MS analysis was performed on an Agilent 1100 series LC/MSD, using automated Agilent HPLC systems, with a gradient of 10-90% B in A over 10 minutes on Phenomenex Columbus C₈, 5μ, 300A, 50×2.0 mm at 0.4 mL/min. Nuclear magnetic resonance (NMR) were obtained on a Bruker DPX400 (400 MHz 1H frequency; QXI probe) in the solvents and temperature indicated. Chemical shifts are expressed in parts per million (δ) and are referenced to residual signals of the solvent. Coupling constants (J) are expressed in Hz.

Solid Phase Chemistry—General Methods

Example inhibitors (1-37) were prepared through a combination of solution and solid phase Fmoc-based chemistries (see ‘Solid Phase Peptide Synthesis’, Atherton, E. and Sheppard, R. C., IRL Press Ltd, Oxford, UK, 1989, for a general description). An appropriately protected and functionalised building block was prepared in solution (e.g. compound (7), Scheme 1), then reversibly attached to the solid phase through an appropriate linker. Rounds of coupling/deprotection/chemical modification e.g. oxidation were then performed until the full length desired molecule was complete (Scheme 2). Example inhibitors (1-37) were then released (cleaved) from the solid phase, analysed, purified and assayed for inhibition verses a range of proteases.

Generally, multipins (polyamide 1.2→10omole loadings, see www.mimotopes.com) were used for the solid phase synthesis, although any suitable solid phase surface could be chosen. In general, the 1.2 μmole gears were used to provide small scale crude examples for preliminary screening, whilst the 10 μmole crowns were used for scale-up synthesis and purification of preferred examples. Standard coupling and Fmoc deprotection methods were employed (see Grabowska, U. et al, J. Comb. Chem. 2(5), 475490, 2000. for a thorough description of solid phase multipin methodologies).

Preparation of Initial Assembly

Building Block-linker constructs (e.g.(9)) were carboxyl activated with 2-(1H-benzotriazole-1-yl)-1,1,3,3-tetramethyluronium hexafluoro phosphate (HBTU, 1 mole equivalent), 1-hydroxybenzotriazole.hydrate (HOBT, 1 mole equivalent) and N-methylrnorpholine (NMM, 2 mole equivalents) in dimethylformamide (DMF, typically 1 to 10 mL) for 5 minutes. Amino functionalised DA/MDA crowns or HEMA gears (10 μmole per crown/1.2 μmole per gear, 0.33 mole equivalent of total surface amino functionalisation compared to activated construct) were added, followed by additional DMF to cover the solid phase surface. The loading reaction was left overnight Following overnight loading, crowns/gears were taken through standard cycles washing, Fmoc deprotection and loading quantification (see Grabowska, U. et al) to provide loaded Building Block-linker constructs (e.g.(10)). Analysis indicated virtually quantitative loading in all examples.

Coupling Cycles

The coupling of standard Fmoc-aminoacids (10 or 20 mole equivalent) were performed via carboxyl activated with 2-(1H-benzotriazole-1-yl)-1,1,3,3-tetramethyluronium hexafluoro phosphate (HBTU, 10 or 20 mole equivalent), 1-hydroxybenzotriazole.hydrate (HOBT, 10 or 20 mole equivalent) and N-methylmorpholine (NMM, 20 or 40 mole equivalents) in dimethylformamide, with pre-activation for 5 minutes. Activated species were dispensed to the appropriate wells of a polypropylene 96-well plate (Beckman, 1 mL wells, 500 μL solution per well for crowns or 250 μL solution per well for gears) in a pattern required for synthesis. Loaded free amino Building Block-linker constructs (e.g.(10)) were added and the coupling reaction left overnight. Following overnight coupling, crowns/gears were taken through standard cycles washing and Fmoc deprotection (see Grabowska, U. et al). Identical activation and coupling conditions were used for the coupling of a range of carboxylic acids (R—COOH). Alternatively, chloroformates e.g. morpholine-4-carbonylchloride (10 mole equivalent), were coupled in DMF with the addition of NMM (10 mole equivalents).

Acidolytic Cleavage Cycle

A mixture of 95% TFA/5% water was predispensed into two polystyrene 96-well plates (Beckman, 1 mL wells, 600 μL solution per well for crowns or 300 μL solution per well for gears) in a pattern corresponding to that of the synthesis. The completed multipin assembly was added to the first plate (mother plate), the block covered in tin foil and cleaved for 2×24 hours. The cleaved multipin assembly was then removed from the first plate and added to the second plate (washing plate) for 15 minutes. The spent multipin assembly was then discarded and the mother/washing plates evaporated on an HT4 GeneVac plate evaporator.

Analysis and Purification of Cleaved Examples

(a) Ex 1.2 μmole Gears. 100 μL dimethylsulphoxide (DMSO) was added to each post cleaved and dried washing plate well, thoroughly mixed, transferred to the corresponding post cleaved and dried mother plate well and again thoroughly mixed. 10 μL of this DMSO solution was diluted to 100 μL with a 90% acetonitrile/10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 nm UV analysis). This provided an approximately 10 mM DMSO stock solution of good quality crude examples for preliminary protease inhibitory screening.

(b) Ex 10 μmole Crowns. 500 μL of a 90% acetonitrile/10% 0.1% aq TFA mixture was added to each washing plate well, thoroughly mixed, transferred to the corresponding mother plate well and again thoroughly mixed. 5 μL of this solution was diluted to 100 μL with a 90% acetonitrile/10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 nm UV analysis). The polystyrene blocks containing crude examples were then lyophilised.

(c) Individual examples (ex (b)) were re-dissolved in a 1:1 mixture of 0.1% aq TFA/acetonitrile (1 mL) and purified by semi-preparative HPLC (Phenomenex Jupiter C₄, 5μ, 300A, 250×10m, a 25-90% B in A gradient over 25 mins, 4.0 mL/min, 215 nm UV detection). Fractions were lyophilised into pre-tarred glass sample vials to provide purified examples (typically 2 to 4 mg, 40 to 80% yield).

(d) Purified examples were dissolved in an appropriate volume of DMSO to provide a 10 mM stock solution, for accurate protease inhibitory screening.

### EXAMPLE 1

### (2R, 3R) 4-tert-Butyl-N-[1-(2,3-dimethyl-4-oxo-tetrahydrofuran-3-yl carbamoyl)2-(4-hydroxyphenyl)-ethyl]-benzamide

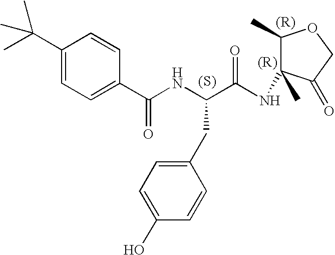

Following the general details from Scheme 1, the required bicycle building block (2R, 3R) (2,3-Dimethyloxo-tetrahydrofuran-3-yl)-carbamic acid 9H-fluoren-9-ylmethyl ester (7a) was prepared as follows.

(1) Preparation of (4S, 5R) 5-Methyl-2-phenyl-4,5-dihydrooxazole-4carboxylic Acid Methyl Ester

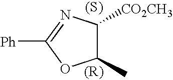

Saturated aqueous sodium hydrogen carbonate (100 mL) was added to a solution of ethyl benzimidate hydrochloride (4 g, 21.25 mmol) in diethyl ether (100 mL). The phases were vigorously mixed and then separated. The ethereal layer was dried (Na₂SO₄) and evaporated under reduced pressure to afford a crude residue. The residue was then dissolved in diethyl ether (8.5 mL) then added to a solution of L-threonine methyl ester hydrochloride (2.1 g, 12.4 mmol) in water (1.5 mL). The mixture was stirred for 20 h then water (20 mL) added. The mixture was then extracted with diethyl ether (40 mL×3) and the combined ethereal layers washed with brine (20 mL), dried (Na₂SO₄) and evaporated under reduced pressure to afford a residue. The excess ethyl benzimidate was removed by distillation at 100° C., under reduced pressure (0.5-1 mbar). The remaining material was purified by flash chromatographed on silica gel (300 g) using ethyl acetate: heptane (1:3) as eluent to afford the (4S, 5R) 5-methyl-2-phenyl-4,5-dihydrooxazole-4-carboxylic acid methyl ester (1.83g, 67%), TLC (single UV spot, R_{f}=0.47, 50% ethyl acetate in heptane), analytical HPLC Rₜ=10.33 min, HPLC-MS (single main UV peak with Rₜ=5.76 min, 220 [M+H]⁺).

δ_{H}(400 z, CDCl₃) 1.59 (3H, d, J=5.6 Hz, 5-CH₃), 3.86 (3H, s, COCH₃), 4.53 (1H, d, J=7.5 Hz, 4-H), 5.02-5.09 (1H, m, 5-H), 7.45-7.57 (3H, m, phenyl CHs′) and 8.03-8.05 (2H, m, phenyl CHs′).

(2) Preparation of (4R, 5R) 4,5-Dimethyl-2-phenyl-4,5-dihydrooxazole-4-carboxylic Acid Methyl Ester

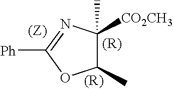

A solution of (4S, SR) 5-methyl-2-phenyl-4,5-dihydrooxazole-4-carboxylic acid methyl ester (8.0 g, 36.53 mmol) in anhydrous tetrahydrofuran (43 mL) was added to a solution of lithium diisopropylamine (20.2 mL, 2M solution in heptanes and tetrahydrofuran) in anhydrous tetrahydrofuran: hexane (243 mL, 10:1) at −78° C. under an atmosphere of nitrogen. After 1 h of stirring at −78° C. iodomethane (5.70 mL, 91.74 mmol) was added. The mixture was stirred for a further 4 h at −78° C. and then allowed to warm to room temperature overnight over 18 h. A saturated aqueous ammonium chloride solution (200 mL) was then added, followed by heptane (300 mL). After thorough mixing, the phases were separated and the organic phase washed with brine, dried (Na₂SO₄) and evaporated under reduced pressure to afford a residue. Flash chromatography over silica (300 g) using ethyl acetate: heptane (3:7) as the eluent afforded the (4R, SR) 4,5-dimethyl-2-phenyl-4,5-dihydrooxazole-4-carboxylic acid methyl ester (7.5 g, 88%), TLC (single UV spot, R_{f}=0.49, 50% EtOAc in heptane), analytical HPLC Rₜ=10.99 min, HPLC-MS (single main UV peak with Rₜ=5.85 min, 234 [M+H]⁺).

δ_{H} (400 MHz, CDCl₃) 1.07 (3H, d, J=6.5 Hz, 5-CH₃), 1.38 (3H, s, 4-CH₃), 3.46 (3H, s, COCH₃), 4.27 (1H, q, J=6.5 Hz, 5-H), 7.12-7.24 (3H, m, phenyl CHs′) and 7.69-7.72 (2H, m, phenyl CHs′).

(3) Preparation of (2R, 3R) 2-Amino-3-hydroxy-2-methylbutyric Acid

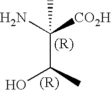

Hydrochloric acid (6 M, 10 mL) was added to (4R, SR) 4,5-dimethyl-2-phenyl-4,5-dihydrooxazole-4-carboxylic acid methyl ester (1.12 g, 4.81 mmol), and the mixture was then heated under reflux for 18 h. The mixture was then cooled to room temperature then water (7 mL) added, followed by diethyl ether (14 mL). The phases were thoroughly mixed and then separated. The aqueous phase was concentrated under reduced pressure then purified over Dowex 50W×4 (activated with 0.01 M HCl). Elution with water (100 mL), followed by 1 M NH₄OH afforded the 2-amino-3-hydroxy-2-methyl-butyric acid (640 mg, 95%). Recrystallisation from H₂O(trace):EtOH-Et₂O afforded the enantiopure (2R, 3R)-isomer (¹H-NMR), TLC (single UV spot, R_{f}=0.27, 4:1:1 ⁿBuOH: H₂O: AcOH), HPLC-MS (single main UV peak with Rₜ=0.45 min. 134 [M+H]⁺).

δ_{H} (400 MHz, D₂O) 0.84 (3H, d, J=6.6 Hz, 4H₃), 1.13 (3H, s, 2-CH₃), 3.69 (1H, q, J=6.6 Hz, 3-H).

(4) Preparation of (2R, 3R) 2-(9H-Fluoren-9-ylmethoxycarbonylamino)-3-hydroxy-2-methylbutyric Acid

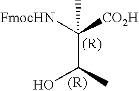

The (2R, 3R) 2-amino-3-hydroxy-2-methylbutyric acid (1.54 g, 11.58 mmol) was added to a vigorously stirred solution of sodium carbonate (3.05 g, 28.94 mmol) in water (66 mL) at 0° C. 1,4-Dioxan (32 mL) was then added, providing an opaque mobile mixture. 9-Fluorenylmethyl chloroformate (3.13 g, 12.16 mmol) in 1,4dioxan (35 mL) was then added over 40 min. The mixture was then allowed to warm to room temperature over 30 mim. Water (300 mL) was then added, and the reaction mixture washed with chloroform (2×250 mL) and the combined organic layers discarded. The aqueous phase was acidified with 1M HCl (pH 2), providing a thick opaque mixture. The acidified aqueous mixture was extracted with chloroform (3×250 mL) and the combined organic phase dried (Na₂SO₄) and evaporated under reduced pressure to afford a residue (4.2 g). Recrystallisation (from ethyl acetate heptane) afforded the (2R, 3R) 2-(9H-fluoren-9-ylmethoxycarbonylamino)-3-hydroxy-2-methylbutyric acid (3.8 g, 92%), TLC (single UV spot, Rf=, 20% MeOH in CHCl₃), analytical HPLC Rₜ=16.09 min, HPLC-MS (single main UV peak with Rₜ=7.41 mins, 356 [M+H]⁺, 378 [M+Na]⁺),

δ_{H} (400 MHz, CDCl₃) 1.17 (3H, d, J=5.6 Hz, 4-H₃), 1.63 (3H, s, 2-CH₃), 4.20-4.30 (2H, m, 3-H and fluorenyl CHCH₂O), 4.44 (2H, br. s, CHCH₂O), 6.09 (1H, s, NH), 7.31-7.35 (2H, m, 2×fluorenyl CH), 7.40-7.43 (2H, m, 2×fluorenyl CB), 7.60 (2H, d, J=7.5 Hz, 2×fluorenyl CH) and 7.78 (2H, d, J=7.5 Hz, 2×fluorenyl CB).

δ_{C} (100 MHz, CDCl₃) 18.6 (C-4), 20.4 (2-CH₃), 47.5 (CHCH₂O), 64.0 (C-2), 67.6 (CH₂O), 71.6 (C-3), 120.5, 125.4, 127.5, 128.2 (8×fluorenyl CHs′), 141.7, 144.0 (4×fluorenyl quaternary Cs′), 157.0 (OCON) and 176.4 (C-1).

(5) Preparation of (1R, 1′R) [3-Diazo-1-(1-hydroxyethyl)-1-methyl-2-oxopropyl]carbamic Acid 9H-fluoren-9-ylmethyl Ester

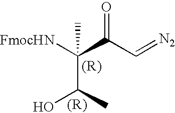

The (2R, 3R) 2-(9H-Fluoren-9-ylmethoxycarbonylamino)-3-hydroxy-2-methylbutyric acid (1.77 g, 5.0 mmol) was dissolved with stirring in anhydrous dichloromethane (30 mL) and tetrahydrofuran (10 mL). The reaction was flushed with nitrogen and cooled to −15° C. iso-Butylchloroformate (0.74 mL, 5.44 mmol) in anhydrous dichloromethane (5 mL) and N-methylmorpholine (1.03 mL, 10.0 mmol) in anhydrous dichloromethane (5 mL) were added simultaneously in 1 mL aliquots over 15 min. Etheral diazomethane [generated from addition of diazald (4.7 g, 15 mmol) in diethyl ether (75 mL) onto sodium hydroxide (5.25 g) in water (7.5 mL)/ethanol (15 mL) at 60° C.] was added to the activated amino acid solution at −20° C. The mixture was then allowed to warm to room temperature and stirred for 20 h. A few drops of acetic acid were added to the mixture. tert-Butylmethylether (100 mL) was then added to the mixture. The ethereal layers were then washed with water (3×75 mL), dried (Na₂SO₄) and the solvents removed under reduced pressure to give a yellow residue (2 g). Flash chromatography of the residue over silica (100g) using gradient elution with ethyl acetate: heptane in the ratios of (1:3) to (1:0) and evaporation of desired fractions in vacuo gave (1R, 1′R) [3-diazo-1-(1-hydroxyethyl)-1-methyl-2-oxopropyl]carbamic acid 9H-fluoren-9-ylmethyl ester, yield 220 mg (11.6%). HPLC-MS (main UV peak with Rₜ=8.84 min, 352.2 [M−N₂+H]⁺, 374.2 [M−N₂+Na]⁺).

(6) Preparation of (2R, 3R) (2,3-Dimethyl-4oxo-tetrahydrofuran-3-yl)-carbamic Acid 9H-fluoren-9-ylmethyl Ester

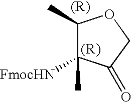

A solution of lithium chloride (247 mg, 5.7 mmol) in water (1.5 mL) and acetic acid (6.0 mL) was added to (1R, 1′R) [3-diazo-1-(1-hydroxyethyl)-1-methyl-2-oxopropyl]carbamic acid 9H-fluoren-9-ylmethyl ester (220 mg, 0.57 mmol). A gas evolved and after 1 h, chloroform (75 mL) was added and the organic phase washed with saturated aqueous sodium hydrogen carbonate (2×70 mL) and brine (70 mL). The chloroform layer was dried (Na₂SO₄) and evaporated under reduced pressure to give a residue (220 mg). Flash chromatography of the residue over silica (35 g) using ethyl acetate: heptane (1:4) as the eluent afforded (21R, 3R) (2,3-dinethyl4-oxotetrahydro-furan-3-yl)carbamic acid 9H-fluoren-9-ylmethyl ester (100 mg, 50%), TLC (single UV spot, R_{f}=0.72, ethyl acetate: heptane 1:1); analytical HPLC Rₜ=18.21 min, HPLC-MS (single main UV peak with Rₜ=8.85 min, 352 [M+H]⁺, 374 [M+Na]⁺).

δ_{H} (400 MHz, CDCl₃) 1.19 (3H, s, 3-CH₃), 1.29 (3H, d, J=6.2 Hz, 2-CH₃), 4.19-4.23 (3H, m, CHCH₂O Fmoc/COCH₂O), 4.40 (2H, d, J=6.6 Hz, CHCH₂O, Fmoc), 4.48 (1HP, q, J=6.2 Hz), 4.97 (1H, s, NH), 7.31-7.70 (8H, m, fluorenyl CHs′).

δ_{C} (100 MHz, CDCl₃) 14.5 (2-CH₃), 16.3 (3-CH₃), 47.5 (u, CHCH₂O, Fmoc), 61.4 (q, Ca), 67.3 (d, CHCH₂O, Fmoc), 70.7 (d, COCH₂O), 77.5 (Cp), 120.4, 125.4, 127.5, 128.2 (8×fluorenyl CHs′), 141.7, 1483.8 and 144.2 (4×fluorenyl quaternary Cs′), 155.1 (OCON) and 214.1 (C4).

Following the general details from Scheme 2, the required bicycle building block (2R, 3R) (2,3-Dimethyloxo-tetrahydrofuran-3-yl)-carbamic acid 9H-fluoren-9-ylmethyl ester (7a) was converted to building block-linker construct (9) as follows:

(2R, 3R) (2,3-Dimethyl-4oxo-tetrahydrofuran-3-yl)-carbamic acid 9H-fluoren-9-ylmethyl ester (7) (78 mg, 0.223 mmol, 1 eq) was dissolved in a mixture of ethanol (3.9 mL) and water (0.56 mL) containing sodium acetate.trihydrate (46 mg, 0.334 mmol, 1.5 eq). 4-[[(hydrazinocarbonyl)amino] methyl]cyclohexanecarboxylic acid. trifluoroacetate (74 mg, 0.223 mmol, 1 eq, Murphy, A. M. et al, J. Am. Chem. Soc., 114, 3156-3157, 1992) was added and the mixture refluxed for 3 days. Chloroform (50 mL) was added and the organics washed with dilute aqueous hydrochloric acid (0.1 M, 2×30 mL), brine (25 mL), dried (Na₂SO₄) and evaporated under reduced pressure to afford linker construct (9) as a white solid (80 mg). Analytical HPLC indicated one main peak at Rₜ=16.99 min for the Z-isomer and another at R. =17.39 min for the E-isomer. BPLC-MS (main UV peaks with Rₜ=8.02 min, 549 [M+H]⁺ for the Z-isomer and Rₜ=8.30 ml, 549 [M+H]⁺ for the E-isomer, Crude (9) was used directly for construct loading.

Following the general details from Scheme 2, the required building block-linker construct (9) was attached to the solid phase providing loaded building block-linker construct (10) as follows:

Building block-linker construct (9) (0.146 mmoles), 2-(1H-benzotriazole-1-yl)-1,1,3,3-tetramethyluroniumhexafluoro phosphate (HBTU, 55 mg, 0.146 mmole), 1-hydroxybenzotriazole.hydrate and (HOBT, 22 mg, 0.146 mmole) were dissolved in dimethylformamide (3 mL) and N-methylmorpholine (NMM, 64 μL, 0.29 mmole) added. After pre-activation for 5 minutes, free amine gears (35×1.2 μmole) were added, followed by dimethylformamide (2.5 mL) and left overnight. The spent coupling solution was then added to free amine crowns (3×10 μmole) and left overnight. Standard washing and analyses indicated loading at approx 70%.

Following the general details from Scheme 2, the required loaded building block-linker construct (10) was elaborated on the solid phase as follows:

Loaded construct (10) was elaborated to EXAMPLE 1 (2R, 3R) 4-tert-Butyl-N-[1-(2,3-dimethyloxo-tetrahydrofuran-3-yl carbamoyl) 2-(4-hydroxyphenyl)-ethyl]-benzamide by standard Fmoc deprotection and sequential coupling with Fmoc-Tyr(OBut)-OH then 4-tert-butylbenzoic acid. The crude example was cleaved and analysed (see general techniques). HPLC Rt=17.80mins (>95%), HPLC-MS 453.2 [M+H]⁺, 927.5 [2M+Na]⁺.

The following examples (2-34) were prepared as detailed for EXAMPLE 1, coupling with the required reagents to provide the full length molecule.

### EXAMPLE 2

### (2R, 3R) Biphenyl-4-carboxylic acid [1-(2,3-dimethyl-4-oxo-tetrahydrofuran-3-ylcarbamoyl)-2-(4-hydroxyphenyl)-ethyl]-amide

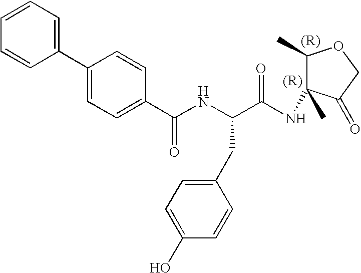

HPLC Rt=17.31 mins (>90%), HPLC-MS 473.2 [M+H]⁺, 967.4 [2M+Na]⁺.

### EXAMPLE 3

### (2R, 3R)N-[1-(2,3-Dimethyloxo-tetrahydrofuran-3-ylcarbamoyl)-2-(4-hydroxyphenyl)etyl]-4-thiophen-2-yl-benzamide

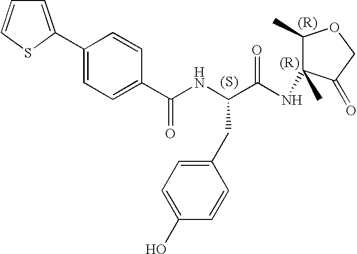

HPLC Rt=16.84mins (>80%), HPLC-MS 479.1 [M+H]⁺, 979.2 [2M+Na]⁺.

### EXAMPLE 4

### (2R, 3R)N-[1-(2,3-Dimethyl-4oxo-tetrahydrofuran-3-ylcarbamoyl)-2-(4-hydroxyphenyl)-ethyl]4-trifluoromethoxy-benzamide

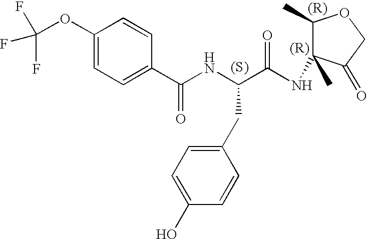

HPLC Rt=16.93mins (>90%), HPLC-MS 481.1 [M+H]⁺.

### EXAMPLE 5

### (2R, 3R)N-[1-(2,3-Dimethyl-4oxo-tetrahydrofuran-3-ylcarbamoyl)-2-(4-hydroxyphenyl)ethyl]-5-thiophen-2-yl-nicotinamide

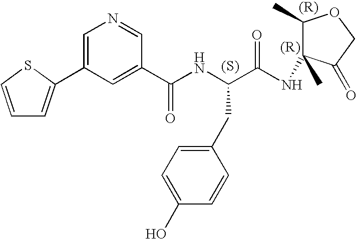

HPLC Rt=12.86mins (>95%), HPLC-MS 480.1 [M+H]⁺, 959.3 [2M+Na]⁺.

### EXAMPLE 6

### (2R, 3R) 2-Pyridin-3-yl-thiazole4-carboxylic acid [1-(2,3-dimethyl-4oxo-tetrahydrofuran-3-ylcarbamoyl)-2-(4-hydroxyphenyl)-ethyl]-amide

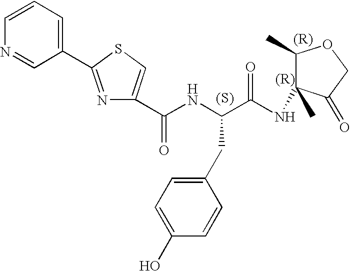

HPLC Rt=9.9/mins (>90%), HPLC-MS 481.1 [M+H]⁺, 961.3 [2M+Na]⁺.

### EXAMPLE 7

### (2R, 3R) 2-Methyl-5-phenyl-furan-3-carboxylic acid [1-(2,3-dimethyl-4-oxo-tetrahydrofuran-3-ylcarbamoyl)-2-(4-hydroxyphenyl)-ethyl]-amide

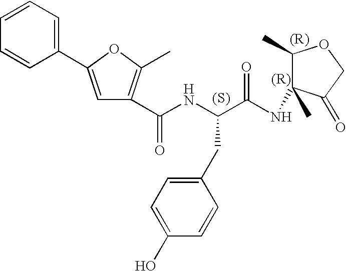

HPLC Rt=17.72 mins (>85%), HPLC-MS 477.2 [M+H]⁺, 975.4 [2M+Na]⁺.

### EXAMPLE 8

### (2R, 3R) 4tert-Butyl-N-[1-(2,3-dimethyl-4-oxo-tetrahydrofuran-3-ylcarbamoyl)-3-methyl-butyl]-benzamide

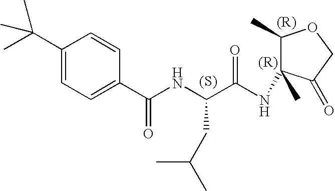

HPLC Rt=19.80mins (>85%), “PLC-MS 403.3 [M+H]⁺, 827.5 [2M+Na]⁺.

### EXAMPLE 9

### (2R, 3R) Biphenyl4-carboxylic acid [1-(2,3-dimethyl-4oxo-tetrahydrofuran-3-ylcarbamoyl)-3-methyl-butyl]-amide

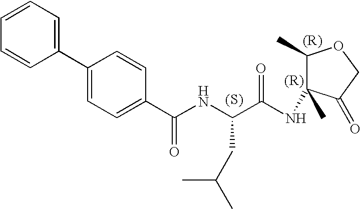

HPLC Rt=19.23mins (>80%), BPLC-MS 423.3 [M+H]⁺, 867.4 [2M+Na]⁺.

### EXAMPLE 10

### (2R, 3R)N-[1-(2,3-Dimethyl-4-oxo-tetrahydrofuran-3-ylcarbamoyl)-3-methyl-butyl]4thiophen-2-yl-benzamide

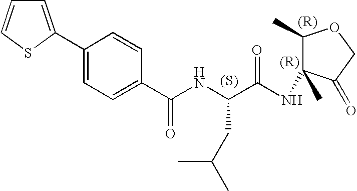

HPLC Rt=18.77mins (>90%), HPLC-MS 429.2 [M+H]⁺, 879.3 [2M+Na]⁺.

### EXAMPLE 11

### (2R, 3R)N-[1-(2,3-Dimethyl-4-oxo-tetrahydrofuran-3-ylcarbamoyl)-3-methyl-butyl]-4-trifluoromethoxy-benzamide

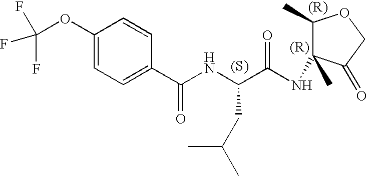

HPLC Rt=18.91 mins (>95%), HPLC-MS 431.2 [M+H]⁺.

### EXAMPLE 12

### (2R, 3R)N-[1-(2,3-Dimethyl-4-oxo-tetrahydrofuran-3-ylcarbamoyl)-3-methyl-butyl]-5-thiophen-2-yl-nicotinamide

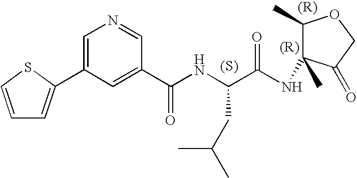

HPLC Rt==14.83 mins (>90%), BPLCMS 430.2 [M+H]⁺, 881.3 [2M+Na]⁺.

### EXAMPLE 13

### (2R, 3R) 2-Pyridin-3-yl-thiazole-4-carboxylic acid [1-(2,3-dimethyl-4-oxo-tetrahydrofuran-3-ylcarbamoyl)-3-methyl-butyl]-amide

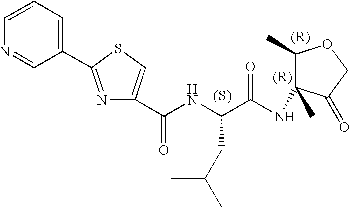

HPLC Rₜ=11.40 mins (>95%), HPLC-MS 431.2 [M+H]⁺, 883.3 [2M+Na]⁺.

### EXAMPLE 14

### (2R, 3R) 2-Methyl-5-phenyl-furan-3-carboxylic acid [1-(2,3-dimethyl-4-oxo-tetrahydrofuran-3-ylcarbamoyl)-3-methyl-butyl]-amide

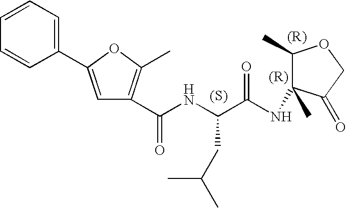

HPLC Rt=19.88mins (>80%), HPLC-MS 427.2 [M+H]⁺, 875.4 [2M+Na]⁺.

### EXAMPLE 15

### (2R, 3R) Furan-3-carboxylic acid [2-benzylsulfanyl-1-(2,3-dimethyl-4-oxo-tetrahydrofuran-3-ylcarbamoyl)-ethyl]-amide

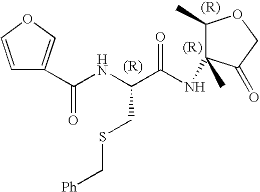

HPLC Rt=15.79 mins (>80%), HPLC-MS 417.0 [M+H]⁺, 855.1 [2M+Na]⁺.

### EXAMPLE 16

### (2R, 3R) Furan-3-carboxylic acid [1-(2,3-dimethyl-4-oxo-tetrahydro-furan-3-ylcarbamoyl)-3-methyl-butyl]-amide

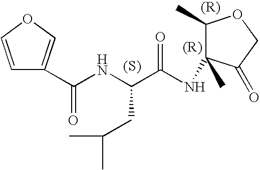

HPLC Rt=12.44 mins (>85%), HPLC-MS 337.1 [M+H]⁺.

### EXAMPLE 17

### (2R, 3R) Furan-3-carboxylic acid [1-(2,3-dimethyloxo-tetrahydro-furan-3-ylcarbamoyl)-2-(4-hydroxyphenyl)-ethyl]-amide

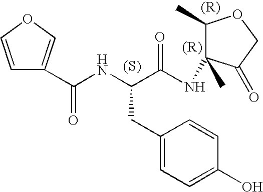

HPLC Rt=10.51mins (>90%), HPLC-MS 387.0 [M+H]⁺.

### EXAMPLE 18

### (2R, 3R) Furan-3-carboxylic acid [1-(2,3-dimethyl-4-oxo-tetrahydro-furan-3-ylcarbamoyl)-3,3-dimethyl-butyl]-amide

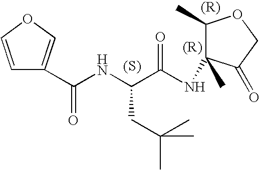

HPLC Rt=14.05mins (>85%), BPLC-MS 351.1 [M+H]⁺, 723.2 [2M+Na]⁺.

### EXAMPLE 19

### (2R, 3R) Furan-3-carboxylic acid [2-cyclohexyl-1-(2,3-dimethyl-oxo-tetrahydrofuran-3-ylcaxbamoyl)-ethyl]-amide

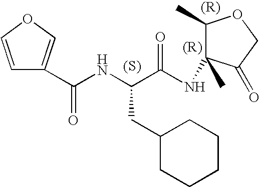

HPLC Rt=16.31 mins (>85%), HPLC-MS 377.1 [M+H]⁺, 775.2 [2M+Na]⁺.

### EXAMPLE 20

### (2R, 3R) Thiophene-3-carboxylic acid [2-benzylsulfanyl-1-(2,3-dimethyloxo-tetrahydrofuran-3-ylcarbamoyl)-ethyl]-amide

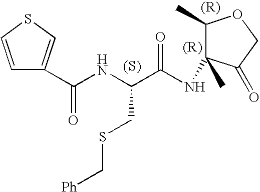

HPLC Rt=16.97 mins (>80% Y), BPLC-MS 433.0 CM+H]⁺, 887.1 [2M+Na]⁺.

### EXAMPLE 21

### (2R, 3R) Thiophene-3-carboxylic acid [1-(2,3-dimethyl-4-oxo-tetrahydrofuran-3-ylcarbamoyl)-3-methyl-butyl]-amide

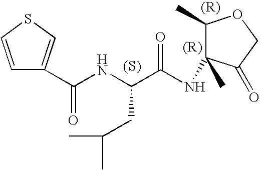

HPLC Rt=13.70mins(>80%),BHPLC-MS353.1 [M+H]⁺, 727.1 [2M+Na]⁺.

### EXAMPLE 22

### (2R, 3R) Thiophene-3-carboxylic acid [1-(2,3-dimethyl-4-oxo-tetrahydrofuran-3-ylcarbamoyl)-2-(4-hydroxyphenyl)-ethyl]-amide

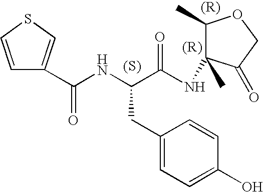

HPLC Rt=11.77 mins (>90%), HPLC-MS 403.0 [M+H]⁺.

### EXAMPLE 23

### (2R, 3R) Thiophene-3-carboxylic acid [1-(2,3-dimethyl-4-oxo-tetrahydrofuran-3-ylcarbamoyl)-3,3diethyl-butyl]-amide

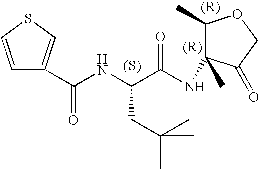

HPLC Rt=15.11 mins (>80%), HPLC-MS 367.1 [M+H]⁺, 755.2 [2M+Na]⁺.

### EXAMPLE 24

### (2R, 3R) Thiophene-3-carboxylic acid [2-cyclohexyl-1-(2,3-dimethyl-4oxo-tetrahydrofuran-3-ylcarbamoyl)-ethyl]-amide

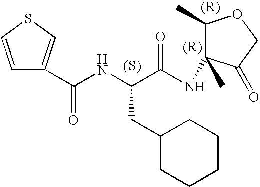

HPLC Rt=17.22mins (>85%), BPLC-MS 393.1 [M+]+, 807.2 [2M+Na]⁺.

### EXAMPLE 25

### (2R, 3R) Naphthalene-1-carboxylic acid [2-benzylsulfanyl-1-(2,3-dimethyl-4-oxo-tetrahydrofuran-3-ylcarbamoyl)-ethyl]-amide

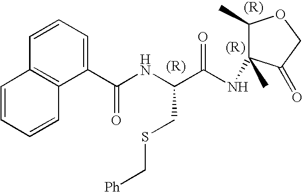

HPLC Rt=19.36 mins (>80%), HPLC-MS 477.1 [M+H]⁺, 975.1 [2M+Na]⁺.

### EXAMPLE 26

### (2R, 3R) Naphthalene-1-carboxylic acid [1-(2,3-dimethyl-4-oxo-tetrahydrofuran-3-ylcarbamoyl)-3-methyl-butyl]-amide

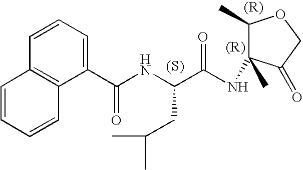

HPLC Rt=16.91 mins (>80%), HPLC-MS 397.1 [M+H]⁺, 815.2 [2M+Na]⁺.

### EXAMPLE 27

### (2R, 3R) Naphthalene-1-carboxylic acid [1-(2,3-dimethyl-4-oxo-tetrahydrofuran-3-ylcarbamoyl)-2-(4-hydroxyphenyl)-ethyl]-amide

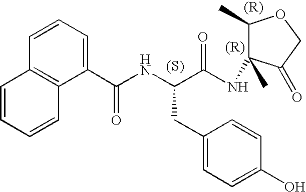

HPLC Rt=14.70mins (>85%), HPLC-MS 447.1 [M+H]⁺.

### EXAMPLE 28

### (2R, 3R) Naphthalene-1-carboxylic acid [1-(2,3-dimethyl-4-oxo-tetrahydrofuran-3-ylcarbamoyl)-3,3-dimethyl-butyl]-amide

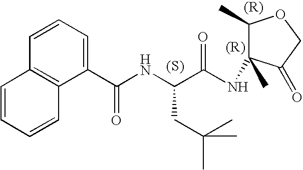

HPLC Rt=17.80 mins (>80%), HPLC-MS 411.1 [M+H]⁺, 843.2 [2M+Na]⁺.

### EXAMPLE 29

### (2R, 3R) Naphthalene-1-carboxylic acid [2-cyclohexyl-1-(2,3-dimethyloxo-tetrahydrofuran-3-ylcabamoyl)-ethyl]-amide

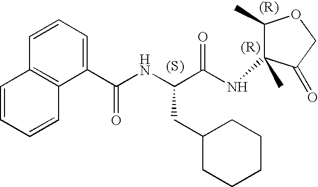

HPLC Rt=19.36mins (>85%), HPLC-MS 437.1 [M+H]⁺, 895.3 [2M+Na]⁺.

### EXAMPLE 30

### (2R, 3R) 2-Benzyloxy-3-cyclohexyl-N-(2,3-dimethyl-4-oxo-tetrahydrofuran-3-yl)-propionamide

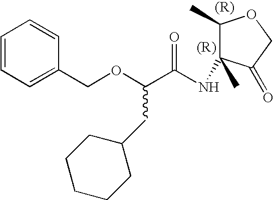

HPLC Rt=21.77 mins (>85%), HPLC-MS 374.2 [M+H]⁺, 769.2 [2M+H]⁺

(a) Preparation of 3-Cyclohexyl-2S-hydroxypropionic Acid (Compound (13) Scheme 4)

A solution of sodium nitrite (12.1g, 175 mmol) in water (40 ml) was added dropwise to a stirred suspension of (S)-α-aminocyclohexanepropionic acid hydrate (5g, 26.5 mmol) in 0.5M sulphuric acid (120 ml, 60 mmol) at 0° C. over 1.5 hours. The mixture was allowed to warm to ambient temperature over 20 hours. The product was extracted into diethyl ether (2×25 ml) then the ethereal layers were washed with saturated aqueous sodium chloride solution (2×25 ml), dried (Na₂SO₄) and the solvents removed in vacuo. The residue (5.3g) was recrystallized from diethyl ether (10 ml) and heptane (25 ml) to give 3-cyclohexyl-2S-hydroxypropionic acid as a white solid, yield 2.4 g, (53%).

δ_{H} (400 MHz, CDCl₃ at 298K), 0.89-1.35 (5H, m) and 1.51-1.86 (7H, m) (OCHCH₂ and cyclohexyl), 4.32 (1H, OCHCH₂, m)

(b)Preparation of 2RS-Benzyloxy-3-cyclohexylpropionic acid (Compound (14) Scheme 4)

Sodium hydride (265 mg of 60% dispersion in oil, 6.6 mmol) was added in two portions to a stirred mixture of 3-cyclohexyl-2S-hydroxypropionic acid (0.52g, 3.0 mmol), dimethylformamide (5 ml) and dichloromethane (5 ml) at 0° C. over 5 minutes. The mixture was stirred at 0° C. for 5 minutes then at ambient temperature for 45 minutes. Benzyl bromide (0.45 ml, 3.8 mmol) was added then the mixture stirred for 1 hour before adding dimethylformamide (5 ml). After stirring for 4 hours potassium iodide (50 mg, 0.3 mmol) was added. The mixture was stirred for 20 hours then heated at 550C for 1 hour then allowed to cool to ambient temperature and poured into water (15 ml). A saturated aqueous sodium chloride solution (5 ml) was added then the mixture was extracted with dichloromethane (5 ml then 10 ml) that was discarded. The aqueous layer was acidified using 1M hydrochloric acid (10 ml) then extracted with dichloromethane (2×10 ml). The dichloromethane layer was dried (MgSO₄) and the solvent removed in vacuo. The residue (0.55 g) was dissolved in dimethylformamide (8 ml) then cooled to 0° C. before adding sodium hydride (190 mg of 60% dispersion in oil, 4.75 mmol). The mixture was stirred for 30 minutes then polymer bound isocyanate (380 mg, 2 mmolNg⁻¹) added. The mixture was stirred for 2 hours at ambient temperature then poured into water (15 ml). 1M Hydrochloric acid (10 ml) was added then the product was extracted into dichloromethane (2×10 ml), dried (Na₂SO₄) and the solvent removed in vacuo. The residue was purified by flash chromatography over silica gel eluting with a gradient of methanol: dichloromethane 0:1→1:20. Appropriate fractions were combined and the solvents removed in vacuo to give 2RS-benzyloxy-3-cyclohexylpropionic acid as a colourless oil, yield 41 mg (5.2%).

HPLC-MS (single main UV peak with Rt=9.47 mins, 261.2 [M−H]⁻; 285.2[M+Na]⁺, 547.3[2M+Na]⁺).

δ_{H} (400 MHz, CDCl₃ at 298K), 0.72-1.03 (2H, cyclohexane, m), 1.08-1.38 (3H, cyclohexane, m), 1.45-1.93 (6H+2Hβ, cyclohexane, m), 3.93-4.18 (1Hα, OCHCO), 4.35-4.53 (1H, CH_{—}₂O, d, J=11.52 Hz), 4.68-4.88 (1H CH₂O, d, J=11.54 Hz), 7.20-7.47 (5H, ArH, m), 9.36 (1H, OH, brs).

Compound (14) was coupled under standard conditions to loaded building block-linker construct (10) (following standard removal of Fmoc), then cleaved to provide

### EXAMPLE 30.

### EXAMPLE 31

### (2R, 3R) 2-(4-tert-Butyl-benzylsulfanyl)-4-methyl-pentanoic Acid (2,3-dimethyl-4-oxo-tetrahydrofuran-3-yl)-amide

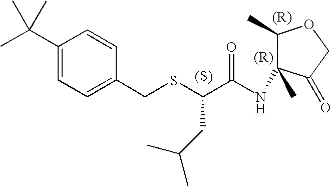

HPLC Rt=24.31 mins (>85%), HPLC-MS 406.2 [M+H]⁺, 833.2 [2M+H]+

(a) Preparation of ZR-Bromo-4-methylpentanoic acid (Compound (16), Scheme 5)

A solution of sodium nitrite (5.1g, 73 mmol) in water (15 ml) was added drop-wise at 0° C. over 5 hours to a stirred mixture of D-leucine (8.75g, 67 mmol), potassium bromide (29.75g, 0.25 mol) and concentrated sulphuric acid (8.6 ml) in water (100 ml). The mixture was stirred for 30 minutes at 0° C. then at ambient temperature for 20 hours. The product was extracted into diethyl ether (2×150 ml) then the combined ethereal layers were washed with saturated aqueous sodium chloride solution (2×100 ml), dried (MgSO₄) and the solvent removed in vacuo. The residue was purified by flash chromatography over silica gel eluting with a gradient of methanol: dichloromethane 1:50→1:20. Appropriate fractions were combined and the solvents removed in vacuo to leave 2R-bromo4methylpentanoic acid (17) as a colourless oil, yield 1.60 g, (12.3%). TLC (single spot, Rf=0.2, methanol: dichloromethane 1:20). Additionally, a second crop (5.2g, 40%) of slightly impure product was obtained

δ_{H} (400 MHz, CDCl₃ at 298K), 0.95 and 0.99 (both 3H, CH₃CH, d, J=6.55 Hz), 1.77-1.89 (1H, CH₃CH, m), 1.93 (2Hβ, m), 4.31 (1Hα, t, J=7.7 Hz), 9.3 (1H, CO₂H, brs).

(b) Preparation of 2S-(4-tert-butylbenzylsulfanyl)-4-methylpentanoic acid (Compound (18), Scheme 5)

A solution of 2R-bromo-4-methylpentanoic acid (compound (16), 1.1 g, 5.6 mmol) and (4-(tert-butyl)phenyl)methanethiol (1.0g, 5.6 mmol) in dimethylformamide (15 ml) was purged with nitrogen for 5 minutes then cooled to 0° C. Triethylamine (0.79 ml, 5.7 mmol) was added drop-wise over 1 minute then the mixture was stirred for two days at ambient temperature. The solvents were removed in vacuo and residue purified by flash chromatography over silica gel eluting with a gradient of methanol: dichloromethane 0:1→1:20. Appropriate fractions were combined and the solvents removed in vacuo to leave a residue which was purified by flash chromatography over silica gel eluting with ethyl acetate: heptane 2:5. Appropriate fractions were combined and the solvents removed in vacuo to give 2S-(4-tert-butylbenzylsulfanyl)-4-methylpentanoic acid (18) as a colourless oil, yield 150 mg, (9%). TLC (single spot, Rf=0.2, heptane: ethyl acetate 5:2), analytical HPLC with main peak Rt=22.117 mins, HPLC-MS (main UV peak with Rt=11.072 mins, 317.2 [M+Na]⁺).

δ_{H} (400 MHz, CDCl₃ at 298K), 0.70 and 0.85 (both 3H, CH₃CH, d, J=6.3), 1.29 (9H, (CH₃)₃C, s), 1.44-1.51 (1H, CH₃CH, m), 1.62-1.75 (2Hβ, m), 3.15-3.20 (1Hα, m), 3.81 and 3.88 (both 1H, SCH₂, d, J=13.2 Hz), 7.25-7.35 (4H, aromatic).

Compound (18) was coupled under standard conditions to loaded building block-linker construct (10) (following standard removal of Fmoc), then cleaved to provide EXAMPLE 31.

### EXAMPLE 32

### (2R, 3R) 24tert-Butyl-phenylmethanesulfonyl)-4-methyl-pentanoic Acid (2,3-diethyl-4-oxo-tetrahydro-furan-3-yl)amide

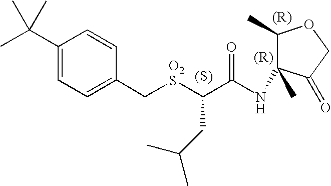

HPLC Rt=22.22 mins (>90%), HPLC-MS 438.1 [M+H]⁺, 897.3 [2M+H]+

Scheme 9. Compound (18) was coupled under standard conditions to loaded building block-linker construct (10). (following standard removal of Fmoc). The intermediate loaded thioether (1.21mmole gear) was oxidised with m-chloroperbenzoic acid (Seq, 65% reagent, 1.6 mg) in dichloromethtne (200 μL) for 5 hrs, followed by standard washing and then cleaved to provide EXAMPLE 32.

### EXAMPLE 33

### (2R, 3R) 2-Cyclohexylmethyl-N-(2,3-dimethyl-4-oxo-tetrahydro-furan-3-yl)-4-morpholin-4-yl-4-oxo-butyramide

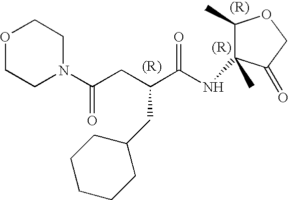

HPLC-MS 395.3 [M+H]⁺, 811.4 [2M+Na]⁺.

(a) Preparation of 2R-cyclohexylmethyl-4-morpholin-4-yl-4-oxo-butyric Acid Methyl Ester.

1-(3-dimethylaminopropyl)-3-ethylcarbodiimide.HCl (1.12g, 5.69 mmol) then 1-hydroxybenzotriazole monohydrate (0.87g, 5.69 mmol) were added to a stirred solution of 2R-(cyclohexylmethyl)succinic acid 1-methyl ester (compound (23),1.0 g, 4.38 mmol) in dimethylformamide (10 ml) at 0° C. under argon. The mixture was stirred for 25 minutes then morpholine (0.7 ml, 8.76 mmol) was added drop-wise over 1 minute and stirring continued at ambient temperature for 16 hours. The product was extracted into ethyl acetate (200 ml) then washed with 1.0M hydrochloric acid (3×100 ml), saturated aqueous sodium hydrogen carbonate solution (3×100 ml), water (100 ml), then saturated aqueous sodium chloride solution (100 ml), dried (MgSO₄), and the solvent removed in vacuo to give 2R-cyclohexylmethyl-4-morpholin-4-yl-4-oxo-butyric acid methyl ester as an off-white solid, yield 1.22g, (94%). HPLC-MS (single peak with Rt=7.91 mins, 298.1 [M+H]⁺, 617.3 [2M+Na]⁺).

(b)Preparation of 2R-cyclohexylmethyl-4-morpholin4-yl-4-oxo-butyric Acid (Compound (24), Scheme 7).

A solution of lithium hydroxide monohydrate (0.51 g, 12.18 mmol) in water (27 ml) was added a stirred solution of 2R-cyclohexylmethyl-4-morpholin-4-yl-4-oxo-butyric acid methyl ester (1.21g,, 4.06 mmol) in tetrahydrofuran (55 ml) and methanol (27 ml) at 0° C. The mixture was stirred at ambient temperature for 1 hours then diluted with water (100 ml). The aqueous layer was extracted with diethyl ether (2×50 ml) which was discarded, then acidified to pH=1-2 with 1M hydrochloric acid. The product was extracted into dichloromethane (3×50 ml), then the combined ethereal layers washed with water (2×50 ml), saturated aqueous sodium chloride solution (2×50 ml), dried (MgSO₄) and the solvent removed in vacuo to leave a residue. The residue was purified by chromatography over silica gel eluting with a gradient of methanol: dichloromethane 1:100→3:100. Appropriate fractions were combined and the solvents removed in vacuo was to give 2R-cyclohexylmethyl-4morpholin-4-yl-4-oxo-butyric acid (24) as a white solid, yield 0.82 g, (71%). HPLC-MS (single peak with Rt=6.769 mins, 284.2 [M+H]⁺, 589.2 [2M+Na]⁺).

δ_{H} (400M, CDCl₃ at 298K),0.77-0.90 (2H, CH₂(cyclohexane), m), 1.05-1.40 (4H, CH₂(cyclohexane),m), 1.50-1.90 (7H, CH(cyclohexane), CH₂(cyclohexane), m), 2.30-2.44 (2Hβ, m), 2.64-2.77 (1Hα, m), 2.96-3.10 (1H, OH, brs), 3.40-3.78 (8H, CH₂OCH₂ and CH₂NCH₂,m).

Compound (24) was coupled under standard conditions to loaded building block-linker construct (10) (following standard removal of Fmoc), then cleaved to provide EXAMPLE 33.

### EXAMPLE 34

### (2R, 3R) 2-Biphenyl-3-yl-4-methyl-pentanoic Acid (2,3-dimethyl-4-oxo-tetrahydrofuran-3-yl)-amide

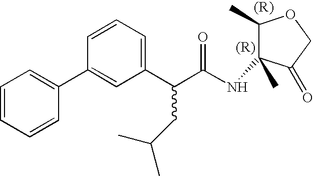

HPLC Rt=21.71 mins (>80%), HPLC-MS 380.2 [M+H]⁺, 781.3 [2M+H]⁺

(a)Preparation of Biphenyl-3-yl-acetic Acid Methyl Ester (Compound (26), Scheme 8)

Concentrated sulphuric acid (588 μL) was added to a solution of 3-bromophenyl acetic acid (10 g, 46.5 mmol) in methanol (100 mL). The mixture was refluxed for 1.5h and then cooled to ambient temperature and evaporated under reduced pressure to afford a residue. The residue was redissolved in diethyl ether (500 mL), washed with water (2×100 mL), brine (100 mL), dried (MgSO₄) and then evaporated under reduced pressure to afford 3-bromophenyl acetic acid methyl ester (10.65 g). The 3-bromophenyl acetic acid methyl ester was dissolved in toluene (17 mL) then phenyl boronic acid (6.8g, 55.69 mmol) added, followed by a aqueous solution of sodium carbonate (93 mL, 2M) and tetkis(triphenylphosphine)palladium (1.6 g, 1.41 mmol). The mixture was stirred overnight then cooled to ambient temperature and an aqueous solution of saturated ammonium chloride (100 mL) added. The mixture was extracted with ethyl acetate (2×200 mL), died (Na₂SO₄) and evaporated under reduced pressure to afford a residue. Flash chromatography of the residue over silica (200 g) using ethyl acetate: heptane (3:48) as the eluent gave biphenyl-3-yl acetic acid methyl ester, yield 10.5 g, (99%), TLC (single UV spot, R_{f}=0.24, 10% ethyl acetate in heptane), analytical BPLC Rₜ=19.55 min, HPLC-MS (single main UV peak with Rₜ=9.35 min, 227.1 [M+H]⁺).

δ_{H} (400 MHz, CDCl₃ at 298K) 3.76 (2H, s, CH₂CO₂CH₃), 3.77 (3H, s, OCH3), 7.34-7.66 (9H, m, biphenyl-3-yl).

(b) Preparation of Biphenyl-3-yl-acetic Acid

Water (39 mL), followed by lithium hydroxide monohydrate (4.2 g, 101.5 mmol) were added to a solution of biphenyl-3-yl acetic acid methyl ester (11.43 g, 50.57 mmol) in methanol (265 mL). The mixture was stirred at ambient temperature for 2h then the organics were removed under reduced pressure. The mixture was acidified with dilute hydrochloric acid (1M, 80 mL), extracted with chloroform (2×100 mL), dried (MgSO₄) and evaporated under reduced pressure to afford biphenyl-3-yl acetic acid as a white solid, yield 10.6g, (99%), analytical HPLC Rₜ=16.565 min, HPLC-MS (single main UV peak with Rₜ=7.91 min, 213.1 [M+H]⁺).

δ_{H} (400 MHz, CDCl₃ at 298K) 3.77 (2H, s, CH₂CO₂CH₃), 7.28-7.52 (9H, m, biphenyl-3-yl).

(c) Preparation of 2RS-Biphenyl-3-yl-methylpent-4-enoic Acid

A solution of biphenyl-3-yl acetic acid (7.0g, 33 mmol) in anhydrous tetrahydrofuran (84 mL) was added dropwise to a solution of lithium diisopropyl amide (36.4 mL, 2M solution in hexanes) in anhydrous tetrahydrofuran (84 mL) at −78° C. The mixture was allowed to warm to 0° C. and stirred for 40 min. The mixture was then cooled to −78° C. and 3-bromo-2-methylpropene (4.97 mL) rapidly added. The mixture was stirred for 1 h at −78° C. then water (28 mL) added and the organics removed under reduced pressure. The mixture was then acidified with hydrochloric acid (6M, 14 ml), extracted with ethyl acetate (3×100 ml), dried (MgSO₄) and evaporated under reduced pressure to afford a residue. Flash chromatography of the residue over silica (400g) using methanol: dichloromethane (3:97) as the eluent afforded impure 2-biphenyl-3-yl-methylpent-4-enoic acid (8.3 g). Flash chromatography over silica (400 g) using methanol: dichloromethane (1.5:98.5) afforded pure 2-biphenyl-3-yl-4-methylpent-4-enoic acid, yield 5.27 g, (60%), TLC (single UV spot, R_{f}=0.28, 5% methanol in dichloromethane), analytical HPLC Rₜ=19.99 min, HPLC-MS (single main UV peak with Rₜ=9.57 min, 267.1 [M+H]⁺).

δ_{H} (400 MHz, CDCl₃ at 298K), 1.765 (3H, s, CH₃),2.53 (1H, dd, J=6.6 and 14.7 Hz, 3-H₁), 2.91 (1H, dd, J=8.9 and 14.7 Hz, 3-H₁), 3.92 (1H, dd, J=6.6 and 8.9 Hz, 2-H), 4.79 (2H, d, J=10.7 Hz, 5-H₂), 7.30-7.62 (9H, m, biphenyl-3-yl).

(d) Preparation of 2RS-Biphenyl-3-yl-4-methylpentanoic acid (Compound (27), Scheme 8)

Palladium on carbon (10%, 300 mg) was added portionwise to a solution of 2RS-biphenyl-3-yl methylpent-4-enoic acid (1 g, 3.76mmol) in ethanol (40 mL) at 0° C. A hydrogen atmosphere was then introduced and the mixture allowed to warm to ambient temperature. The mixture was stirred for 18h, then the hydrogen atmosphere removed and the mixture filtered over Celite and the catalyst washed with ethanol (40 mL). The combined organic filtrate was concentrated under reduced pressure to afford a residue, which was flash chromatographed over silica (150g) using methanol:dichloromethane (1:99) as the eluent to afford 2RS-biphenyl-3-yl-4-methylpentanoic acid, yield 980 mg, (98%), TLC (single UV spot, R_{f}=0.45, 5% methanol in dichloromethane), analytical HPLC Rₜ=20.92 min, HPLC-MS (single main UV peak with Rₜ=10.15 min, 269.1 [M+H]⁺, 291.1 [M+Na]⁺).

δ_{H} (400 MHz, CDCl₃ at 298K), 0.93 (6H, d, J=6.6 Hz, 2×CH₃), 1.52-1.57 (1H, m, 4H₁), 1.71-1.76 (1H, m, 3-H₁), 1.97-2.05 (1H, m, 3-H₁), 3.66 (1H, t, J=7.8 Hz, 2-H₁), 7.32-7.60 (9H, m, biphenyl-3-yl).

Compound (27) was coupled under standard conditions to loaded building block-linker construct (10), then cleaved to provide EXAMPLE 34.

### EXAMPLE 35

### (2S, 3R) 4-tert-Butyl-N-[1-(2,3-dimethyl-4-oxo-tetrahydrofuran-3-yl carbamoyl) 2-(4-hydroxyphenyl)-ethyl]-benzamide

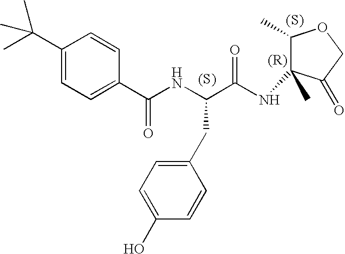

Following the general details from Scheme 1, the required bicycle building block (2S, 3R) (2,3-Dimethyloxo-tetrahydrofuran-3-yl)-carbamic acid 9H-fluoren-9-ylmethyl ester (7b) was prepared as follows.

(1) Preparation of (2S) 2-tert-Butoxycarbonylamino-3-phenylpropionic Acid 1-methyl-2-oxopropyl Ester

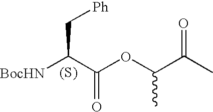

1-[3(imethylamino)propyl]-3-ethylcarbodiimide hydrochloride (10.9 g, 57 mmol) and 4-(diinethylamino)pyridine (700 mg, 5.7 mmol) were added to a solution of (2R)-2-tert-butoxycarbonylamino-3-phenylpropionic acid (11 g, 41.8 mmol) in anhydrous dichloromethane (20 mL) at 0° C. and under a nitrogen atmosphere. The mixture was stirred for 5 min and then 3-hydroxybutan-2-one (6.6 g, 75 mmol, assuming monomeric form) was added at 0° C. The mixture was allowed to warm to room temperature then stirred for 3 days. The mixture was then evaporated under reduced pressure to afford a residue. Flash chromatography of the residue over silica gel (500 g) using ethyl acetate: heptane (3:7) as the eluent afforded (2S) 2-tert-butoxycarbonylamino-3-phenylpropionic acid 1-methyl-2oxopropyl ester (10.9 g, 78%), TLC (single UV spot, R_{f}=0.40, 50% ethyl acetate in heptane), and HPLC-MS (single main UV peak with Rₜ=9.16 min, 358.2 [M +Na]⁺, 693.3 [2×M+Na]⁺)

(2) Preparation of (2S, 3S, 5S) 5-Benzyl-2,3-dimethyl-6-oxomorpholine-3-carbonitrile

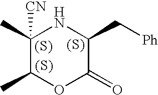

Trifluoroacetic acid (77 mL) was added to a solution of (2R) 2-tert-butoxycarbonylamino-3-phenylpropionic acid 1-methyl-2-oxopropyl ester (7.99 g, 23.8 mol) in anhydrous dichloromethane (140 mL) at 0° C. under a nitrogen atmosphere. The mixture was stirred for 1 h at 0° C. then evaporated under reduced pressure to afford a residue. Toluene (40 mL) was then added to the residue and evaporated under reduced pressure. This procedure was repeated twice, to remove excess trifluoroacetic acid. The residue was then dissolved in anhydrous 2-propanol (390 mL) under a nitrogen atmosphere. Powdered sodium cyanide (2.6 g, 47.6 mol) was then added in one portion and the mixture stirred for 2 h at ambient temperature. The mixture was then rapidly filtered through anhydrous sodium sulfate and concentrated under reduced pressure. Flash chromatography over silica gel (200 g) using ethyl acetate: heptane (3:7) as eluent afforded crude (2S, 3S, 5S) 5-benzyl-2,3-dimethyl-6-oxo-morpholine-3-carbonitrile (2.7 g, 46%). Recrystallisation (diethyl ether-heptane) afforded crystalline material (1.05 g), TLC (single Uw spot, R_{f}=0.38, 50% ethyl acetate in heptane), analytical HPLC R. =15.94 min, HPLC-MS (single main Uw peak with Rₜ=7.53 min, 245.1 [M+H]⁺, 267.1 [M+Na]⁺).

δ_{H} (400 MHz, CDCl₃) 1.35 (3H, d, J=6.5 Hz, 2-CH₃), 1.48 (3H, s, 3-CH₃), 3.01 (1H, dd, J=9.1 and 13.8 Hz, CH₂Ph), 3.48 (1H, dd, J=3.6 and 13.8 Hz, CH₂Ph), 4.17 (1H, dd, J=3.6 and 9.1 Hz, 5-H), 4.57 (1H, q, J=6.5 Hz, 2-H), 7.29-7.42 (5H, m, C₆H₅).

(3) Preparation of (2R, 3S) 2-Amino-3-hydroxy-2-methylbutyric Acid

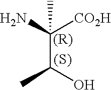

To a solution of (2S, 3S, 5S) 5-benzyl-2,3-dimethyl-6-oxomorpholine-3-carbonitrile (1.38 g, 5.63 mmol) in anhydrous diethyl ether (120 mL) was added tert-butyl hypochlorite (1.26 mL, 11.25 mmol) at 0° C. under a nitrogen atmosphere. The solution was stirred at room temperature for 2 h, then triethylamine (1.26 mL) was added at 0° C. The mixture was then allowed to warm to room temperature and stirred for 20 h. The resulting suspension was filtered and the solid residue washed with diethyl ether (20 mL). Water (100 mL) was then added to the filtrate and the product extracted with ether (50 mL×3). The combined ethereal layers were dried (MgSO₄) and evaporated under reduced pressure to afford a residue (1.9 g). Concentrated hydrochloric acid (70 mL) was then added to the residue at 0° C. The mixture was stirred for 1 h at 0° C. then at room temperate for 24 h. The mixture was then transferred to a sealed pressure tube and heated at 80° C. for 2 days. The mixture was then cooled and extracted with diethyl ether (50 mL×3). The aqueous layer was then concentrated under reduced pressure then purified over Dowex50WX4 (activated with 0.01 M hydrochloric acid). Elution with water (until the eluent approached ˜pH=7), followed by ammonium hydroxide (1 M) afforded impure (2R, 3s) 2-amino-3-hydroxy-2-methylbutyric acid (555 mg). Recrystallisation from H₂O(trace);EtOH-Et₂O afforded the pure amino acid (250 mg), TLC (single UV spot, R_{f}=0.27, 4:1:1 ⁿBuOH:H₂O:AcOH), HPLC-MS (single main UV peak with Rₜ=0.45 min, 134 [M+H]³⁰ ).

δ_{H} (400 M, D₂O) 0.92 (3H, d, J=6.6 Hz, 4-H₃), 1.06 (3H, s, 2-CH₃), 3.85 (1H, q, J=6.6 Hz, 3-H).

(4) Preparation of (2R, 3S) 2-(9H-Fluoren-9-ylmethoxycarbonylamino)-3-hydroxy-2-methylbutyric Acid

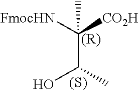

The (2R, 3S) 2-amino-3-hydroxy-2-methylbutyric acid (288 mg, 2.17 mmol) was added to a vigorously stirred solution of sodium carbonate (570 mg, 5.41 mmol) in water (12 mL) at 0° C. 1,4-Dioxan (6 mL) was then added, providing an opaque mobile mixture. 9-Fluorenylmethyl chloroformate (585 mg, 2.27 mmol) in 1,4-dioxan (6 mL) was then added over 40 min. The mixture was then allowed to warm to room temperature over 40 min. Water (100 mL) was then added, the reaction mixture washed with chloroform (100 mL) and the organic layer discarded. The aqueous phase was acidified with 1M HCl (pH 2), providing a thick opaque mixture. The acidified aqueous mixture was extracted with chloroform (5×80 mL) and the combined organic phase dried (Na₂SO₄) and evaporated under reduced pressure to afford (2R, 3s) 2-(9H-Fluoren-9-ylmethoxycarbonylamino)-3-hydroxy-2-methylbutyric acid (616 mg, 80%), TLC (single UV spot, R_{f}=0.24, 20% methanol in chloroform), analytical HPLC Rₜ=15.54 min and HPLC-MS (single main UV peak with Rₜ=7.70 min. 356.2 [M+H]⁺, 378.1 [M+Na]⁺).

(5) Preparation of (2R, 3s) 2-(9H-Fluoren-9-ylmethoxycarbonylamino)-3-hydroxy-2-methylbutyric Acid Alkyl Ester

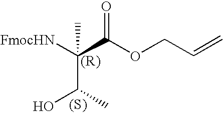

A solution of (616 mg, 1.74 mmol) and tricaprylmethylammonium chloride (701 mg, 1.74 mmol) in dicbloromethane (2.6 mL) was added to a stirred solution of sodium hydrogen carbonate (146 mg, 1.74 mmol) in water (2.6 mL), then alkyl bromide (0.15 mL, 1.74 mmol) was added in one portion. The biphasic mixture was vigorously stirred for 5 days then diluted with water (40 mL) and the product extracted into dichloromethane (3×80 mL). The combined organic layers were dried (Na₂SO₄) and evaporated under reduced pressure to afford a residue (1.1 g). Flash chromatography of the residue over silica gel (200 g) using ethyl acetate: heptane (3:7) as the eluent afforded (2R, 3S) 2-(9H-fluoren-9-ylmethoxycarbonylamino)-3-hydroxy-2-methyl butyric acid alkyl ester (360 mg, 52%), TLC (single UV spot, R_{f}=0.48, 50% ethyl acetate in heptane), analytical HPLC R. =18.42 min and HPLC-MS (single main UV peak with Rₜ=9.30 min, 396.2 [M+H]⁺, 418.2 [M+Na]⁺).

δ_{H} (400 M, CDCl₃) 1.21 (3H, d, J=6.2 Hz, 4-H₃), 1.52 (3H, s, 2-CH₃), 3.38 (1H, br. s, OR), 3.47-4.16 (1H, m, 3-H), 4.25 (1H, t, J=6.8 Hz, fluorenyl CHCH₂O), 4.38-4.42 (2H, m, fluorenyl CHCH₂O), 4.67 (2H, br. s, OCH₂CHCH₂), 5.24 (1H, dd, J=1.2 and 10.44 Hz, OCH₂CHCH₂), 5.35 (1H, dd, J=1.1 and 10.44 Hz, OCH₂CHCH₂), 5.52 (1H, s, NB), 5.87-5.95 (1H, m, OCH₂CHCH₂), 7.31-7.35 (2H, m, 2×fluorenyl CB), 7.40-7.43 (2H, m, 2×fluorenyl CB), 7.60 (2H, d, J=7.4 Hz, 2×fluorenyl CB) and 7.78 (2H, d, J=7.5 Hz, 2×fluorenyl CH).

(6) Preparation of (2R, 3S) 3-tert-Butoxy-2-(9H-fluoren-9-ylmethoxy carbonylamino)₂-methylbutync Acid Allyl Ester

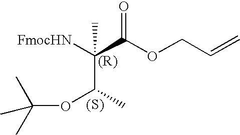

A stirred solution of (2R, 3s) 2-(9H-fluoren-9-ylmethoxycarbonylamino)-3-hydroxy-2-methylbutyric acid allyl ester (312 mg, 0.69 mmol) in dichloromethane (10 mL) was cooled in a sealed pressure vessel to −780C then isobutylene gas (˜8 mL) condensed into the solution. Concentrated sulphuric acid (27 μL) was added then the pressure vessel sealed. The mixture was stirred at ambient temperature for 3 days then cooled to −78° C. N-Methylmorpholine (56 μL) was added then the unsealed pressure vessel allowed to warm to ambient temperature. The mixture was diluted with saturated aqueous sodium hydrogen carbonate solution (100 mL) then the product extracted into dichloromethane (3×100 mL). The combined dichloromethane layers were washed with brine (50 mL), dried (Na₂SO₄) and evaporated under reduced pressure to afford a residue. Flash chromatography of the residue over silica gel (35 g) using ethyl acetate: heptane (3:7) as the eluent afforded (2R, 3S) 3-tert-butoxy-2-(9H-fluoren-9-ylmethoxy carbonylamino)-2-methyl butyric acid allyl ester (310 mg, 99%), TLC (single UV spot, R_{f}=0.70, 50% ethyl acetate in heptane), analytical HPLC Rₜ=23.59 min and HPLC-MS (single main UV peak with Rₜ=12.09 min, 474.1 [M+Na]⁺).

δ_{H} (400 MHz, CDCl₃) 1.16-1.25 (12H, m, 4-H₃ and C(CH₃)₃), 1.62 (3H, s, 2-CH3), 3.95-4.05 (1H, m, 3-H), 4.234.43 (3H, m, fluorenyl CHCH₂O and fluorenyl CHCH₂O), 4.554.71 (2H, m, OCH₂CHCH₂), 5.21 (1H, ddd, J=1.2, 2.5 and 17.2 Hz, OCH₂CHCH₂), 5.34 (1H, d, J=17.2 Hz, OCH₂CHCH₂), 5.72 (1H, s, NH), 5.86-5.95 (1H, m, OCH₂CHCH₂), 7.31-7.35 (2H, m, 2×fluorenyl CH), 7.40-7.43 (2H, m, 2×fluorenyl CB), 7.62-7.70 (2H, m, 2×fluorenyl CB) and 7.78 (2H, d, J=7.5 Hz, 2×fluorenyl CH).

(7) Preparation of (2R, 3S) 3-tert-Butoxy-2-(9H-fluoren-9-ylmethoxy carbonylamino)-2-methylbutyric Acid

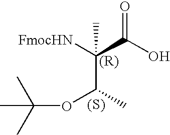

Tetrakistriphenylphosphine palladium(0) (17.25 mg, 0.015 mmol), dichloromethane (10 mL) then phenyltrihydrosilane (0.18 mL, 1.33 mmol) were added consecutively to (2R, 3S) 3-tert-butoxy-2-(9H-fluoren-9-ylmethoxycarbonylamino)-2-methyl butyric acid allyl ester (300 mg, 0.665 mmol) under nitrogen. The mixture was stirred for 90 min then absorbed onto silica gel (2 g). Flash chromatography of the absorbed residue over silica gel (35 g) using ethyl acetate: heptane (2:3), followed by (1:1) as the eluent afforded (2R, 3S) 3-tert-butoxy-2-(9H-fluoren-9-ylmethoxycarbonylamino)-2-methyl butyric acid (140 mg, 51%), TLC (single WV spot, R_{f}=0.33, 50% ethyl acetate in heptane), analytical HPLC Rₜ=20.20 min and HPLC-MS (single main V peak with Rₜ=10.25 min, 434.1 [M+Na]⁺).

δ_{H} (400 Mz CDCl₃) 1.19 (3H, d, J=6.1 Hz, 4-H₃), 1.32 (9H, s, C(CH₃)₃), 1.69 (3H, s, 2-CH₃), 4.23 (1H, t, J=7.0 Hz, fluorenyl CHCH₂O), 4.37 (2H, d, J=7.0 Hz, fluorenyl CHCH₂O), 4.61-4.65 (1H, m, 3-H), 6.12 (1H, s, NH), 7.31-7.34 (2H, m, 2×fluorenyl CH), 7.39-7.43 (2H, m, 2×fluorenyl CH), 7.59-7.62 (2H, m, 2×fluorenyl CH) and 7.77 (2H, d, J=7.5 Hz, 2×fluorenyl CH).

δ_{C} (100 MHz, CDCl₃) 18.3 (C-4), 21.3 (2-CH3), 28.7 (C(CH₃)₃), 47.2 (CHCH₂O), 60.5 (C(CH₃)₃), 62.7 (C-2), 66.7 (CH₂O), 69.5 (C-3), 120.1, 125.2, 127.2, 127.8 (8×fluorenyl CHs′), 141.4, 143.8 (4×fluorenyl quaternary Cs′), 154.6 (OCON) and 174.8 (C-1).

(8) Preparation of (1R, 2S) (2-tert-Butoxy-1-fluorocarbonyl-1-methyl propyl)-carbamic Acid 9H-fluoren-9-ylmethyl Ester

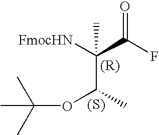

Pyridine (47 μL, 0.55 mmol) then cyanuric fluoride (63 μL, 0.71 mmol) were added consecutively at 0° C. to a stirred solution of (2R, 3S) 3-tert-butoxy-2-(9H-fluoren-9-ylmethoxycarbonylamino)-2-methyl butyric acid (130 mg, 0.316 mmol) in dichloromethane (8 mL) under nitrogen. The suspension was slowly warmed to ambient temperature and stirred for 20 h. Crushed ice (˜10 ml) and ice-chilled water (10 ml) was added, then the product was extracted into dichloromethane (40 ml). The dichloromethane layer was dried (MgSO₄) and evaporated under reduced pressure to afford (1R, 2S) (2-tert-butoxy-1-fluorocarbonyl-1-methylpropyl)-carbamic acid 9H-fluoren-9-ylmethyl ester (138 mg), TLC (single UV spot, R_{f}=0.75, 50% ethyl acetate in heptane), HPLC-MS (single main UV peak with Rₜ=11.66 min, 436.1 [M+Na]⁺).

(9) Preparation of (1R, 1′S) [1-(1-tert-Butoxyethyl)-3-diazo-1-methyl-2-oxo propyl]-carbamic Acid 9H-fluoren-9-ylmethyl Ester

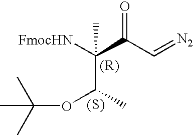

Ethereal diazomethane [generated from diazald (˜15 mmol) addition in diethyl ether (75 mL) to sodium hydroxide (5.25 g) in water (7.5 mL)/ethanol (15 mL) at 60° C. and dried over potassium hydroxide pellets] was added to a stirred solution of (1R, 2S) (2-tert-butoxy-1-fluorocarbonyl-1-methylpropyl)-carbamic acid 9H-fluoren-9-ylmethyl ester (138 mg, 0.316 mmol) in dichloromethane (3 mL) at 0° C. The mixture was then allowed to warm to ambient temperature and stirred for 20 hours. A few drops of acetic acid were added to quench any excess diazomethane then the solution was stirred for 5 minutes before adding tert-butyl methyl ether (100 mL). The ethereal layer was washed with saturated aqueous sodium hydrogen carbonate (2×50 mL), dried (Na₂SO₄) and evaporated under reduced pressure to afford a residue. Flash chromatography of the residue over silica gel (35 g) using ethyl acetate : heptane (0.1:19) as the eluent afforded (4R, 4′S) 4-(1-tert-Butoxyethyl)-2-(9H-fluoren-9-ylmethoxy)-4-methyl-4H-oxazol-5-one (55 mg, 45%), TLC (single UV spot, R_{f}=0.77, 50% ethyl acetate in heptane), analytical HPLC Rₜ=21.15 min and HPLC-MS (single main UV peaks at Rₜ=10.42 min, 434.2 [M+H₂O+Na]⁺ and Rₜ=11.65 min, 434.2 [M+H₂O+Na]⁺).

δ_{H} (400 MHz, CDCl₃) 1.13 (9H, s, C(CH₃)₃), 1.25 (3H, d, J=6.1 Hz, CH₃CH), 1.35 (3H, s, 4-CH₃), 3.81 (1H, q, J=6.1 Hz, CHCH₃), 4.41 (1H, dd, J=8.1 and 9.8 Hz, fluorenyl CHCH₂O), 4.50 (1H, m, fluorenyl CHCH₂O), 4.80 (1H, dd, J=6.2 and 9.8 Hz, fluorenyl CHCH₂O), 7.31-7.35 (2H, m, 2×fluorenyl CH), 7.41-7.45 (2H, m, 2×fluorenyl CH), 7.67 (2H, d, J=7.5 Hz, 2×fluorenyl CH) and 7.79 (2H, d, J=7.5 Hz, 2×fluorenyl CB). 5c (100 MHz, CDCl₃) 17.3 (4′-CH₃), 20.5 (4-CH₃), 28.8 (C(CH₃)₃), 46.3 (CHCH₂O), 71.1 (C-4), 72.1 (CH₂O), 74.4 (CHCH₃), 75.6 (C(CH₃)₃), 120.2, 125.4, 125.7, 127.3, 128.0, 128.1 (8×fluorenyl CHs′), 141.4, 141.5, 143.0, 143.6 (4×fluorenyl quaternary Cs′), 157.6 (OCON) and 178.8 (C-5),

Further elution gave a mixed fraction containing the above oxazolone and (1R, 1′s) [1-(1-tert-Butoxyethyl)-3-diazo-1-methyl-2-oxo propyl]-carbamic acid 9H-fluoren-9-ylmethyl ester [TLC (UV spot, R_{f}=0.75, 50% ethyl acetate in heptane] and 3-tert-Butoxy-2-(9H-fluoren-9-ylmethoxycarbonylamino)-2-methylbutyric acid methyl ester (35 mg).

(10) Preparation of (2S, 3R) (2,3-Dimethyl-4-oxo-tetrahydrofuran-3-yl)-carbamic Acid 9H-fluoren-9-ylmethyl Ester

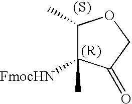

A solution of lithium chloride (21.7 mg, 0.51 mmol) in water (0.25 mL) and acetic acid (1 mL) was added to the above mixed fraction (35 mg) containing (1R, 1′s) [1-(1-tert-Butoxyethyl)-3-diazo-1-methyl-2-oxopropyl]-carbamic acid 9H-fluoren-9-ylmethyl ester. A gas evolved and after 1 h, chloroform (40 mL) was added and the organic phase washed with saturated aqueous sodium hydrogen carbonate (20 mL). The chloroform layer was dried (Na₂SO₄) and evaporated under reduced pressure to give a residue (40 mg). Flash chromatography of the residue over silica (20 g) using ethyl acetate: heptane (1:4), followed by (1:1) as the eluent afforded (2S, 3R) (2,3-dimethyl-4-oxotetrahydrofuran-3-yl)carbamic acid 9H-fluoren-9-ylmethyl ester (6 mg), TLC (single WV spot, R_{f}=0.57, ethyl acetate: heptane 1:1); analytical HPLC R_{f}=18.22 min and HPLC-MS (single main UV peak with Rₜ=8.92 in, 352 [M+H]⁺, 374 [M+Na]⁺).

δ_{H} (400 MHz, CDCl₃) 1.12 (3H, d, J=4.3 Hz, 2-CH₃), 1.50 (3H, s, 3-CH₃), 4.05 (1H, d, J=17.9 Hz, 5-H), 4.16-4.24 (2H, m, 5-H and fluorenyl CHCH₂O), 4.384.48 (2H, m, CHCH₂O), 4.50-4.65 (1H, br. s, 2-H), 4.84 (1H, s, NR), 7.31-7.33 (2H, m, 2×fluorenyl CR), 7.39-7.43 (2H, m, 2×fluorenyl CB), 7.58 (2H, d, J=7.4 Hz, 2×fluorenyl CB) and 7.78 (2H, d, J=7.4 Hz, 2×fluorenyl CB)

Following the general details from Scheme 2, the required bicycle building block (2S, 3R) (2,3-Dimethyl-4oxo-tetrahydrofuran-3-yl)-carbamic acid 9H-fluoren-9-ylmethyl ester (7b) was converted to building block-linker construct (9) as follows:

(2S, 3R) (2,3-Dimethyl 4oxo-etrtydrofuran-3-yl)-carbamic acid 9H-fluoren-9-ylmethyl ester (7b) (8 mg, 0.0214 mmol, 1 eq) was dissolved in a mixture of ethanol (1.86 mL) and water (0.27 mL) containing sodium acetate.trihydrate (22 mg, 0.1605 mmol, 7.5 eq). 4-[[(hydrazinocarbonyl)amino] methyl]cyclohexanecarboxylic acid. trifluoroacetate (35 mg, 0.1605 mmol, 7.5 eq, Murphy, A. M. et al, J. Am. Chem. Soc., 114, 3156-3157, 1992) was added and the mixture refluxed for 2 days. Chloroform (50 mL) was added and the organics washed with dilute aqueous hydrochloric acid (0.1 M, 2×30 mL), brine (25 mL), dried (Na₂SO₄) and evaporated under reduced pressure to afford linker construct (9) as a white solid (10 mg). Analytical HPLC indicated one main peak at Rₜ=17.53 min for the Z-isomer and another at Rₜ=17.88 min for the E-isomer. HPLC-MS (main UV peaks with Rₜ=8.37 min, 549 [M+H]⁺ for the Z-isomer and Rₜ=8.98 min, 549 [M+H]⁺ for the E-isomer, Crude (9) was used directly for construct loading.

Following the general details from Scheme 2, the required building block-linker construct (9) was attached to the solid phase providing loaded building block-linker construct (10) as follows:

Building block-linker construct (9) (10.6 mg, 0.0193 mmole), 2-(1H-benzotriazole-1-yl)-1,1,3,3-tetramethyluroniumhexafluoro phosphate (HBTU, 7.3 mg, 0.0193 mmole), 1-hydroxybenzotriazole.hydrate and (HOBT, 2.96 mg, 0.0193 mmole) were dissolved in dimethylformamide (0.5 mL) and N-methylmorpholine (NMM, 4.2 μL, 0.0386 mmole) added. After pre-activation for 5 minutes, free amine gears (6×1.2 μmole) were added, followed by dimethylformamide (1.0 mL) and left overnight The spent coupling solution was then added to free amine crown (1×10 μmole) and left overnight. Standard washing and analyses indicated loading at approx 70%.

Following the general details from Scheme 2, the required loaded building block-linker construct (10) was elaborated on the solid phase as follows:

Loaded construct (10) was elaborated to EXAMPLE 35 (2S, 3R) 4-tert-Butyl-N-[1-(2,3-dimethyl-4-oxo-tetrahydrofuran-3-yl carbamoyl) 2-(4-hydroxyphenyl)-ethyl]-benzamide by standard Fmoc deprotection and sequential coupling with Fmoc-Tyr(OBut)-OH then 4-tert-butylbenzoic acid. The crude example was cleaved and analysed (see general techniques). HPLC Rₜ=17.31mins (>90%), HPLC-MS 453.2 [M+H]⁺.

The following examples (36-37) were prepared as detailed for EXAMPLE 35, coupling with the required reagents to provide the fulll length molecule.

### EXAMPLE 36

### (2S, 3R) 4-tert-Butyl-N-[1-(2,3-dimethyl-4-oxo-tetrahydrofuran-3-ylcarbamoyl)-3-methyl-butyl]-benzamide

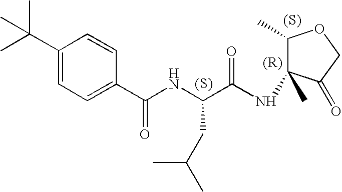

HPLC Rₜ=19.4 mins (>90%), HPLC-MS 403.3 [M+H]⁺, 827.5 [2M+Na]⁺.

### EXAMPLE 36

### (2S, 3R) Furan-3-carboxylic acid [2-cyclohexyl-1-(2,34dimethyl-4-oxo-tetrahydrofuran-3-ylcarbamoyl)-ethyl]-amide

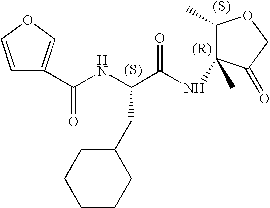

HPLC Rₜ=16.3 mins (>90%), HPLC-MS 377.2 [M+H]⁺, 775.4 [2M+Na]⁺.

### EXAMPLE A

### Assays for Cysteine Protease Activity

The compounds of this invention may be tested in one of a number of literature based biochemical assays that are designed to elucidate the characteristics of compound inhibition. The data from these types of assays enables compound potency and the rates of reaction to be measured and quantified. This information, either alone or in combination with other information, would allow the amount of compound required to produce a given pharmacological effect to be determined.

General Materials and Methods

Unless otherwise stated, all general chemicals and biochemicals were purchased from either the Sigma Chemical Company, Poole, Dorset, U.K. or from Fisher Scientific UK, Loughborough, Leicestershire, U.K. Absorbance assays were carried out in flat-bottomed 96-well plates (Spectra; Greiner Bio-One Ltd., Stonehouse, Gloucestershire, U.K.) using a SpectraMax PLUS384 plate reader (Molecular Devices, Crawley, U.K.). Fluorescence high throughput assays were carried out in either 384-well microtitre plates (Corning Costar 3705 plates, Fisher Scientific) or 96-well ‘U’ bottomed Microfluor W1 microtitre plates (Thermo Labsystems, Ashford, Middlesex, U.K.). Fluorescence assays were monitored using a SpectraMax Gemini fluorescence plate reader (Molecular Devices). For substrates employing either a 7-amino-4methylcoumarin (AMC) or a 7-amino-4-trifluoromethylcoumarin (AFC) fluorophore, assays were monitored at an excitation wavelength of 365 nm and an emission wavelength of 450 nm and the fluorescence plate reader calibrated with AMC. For substrates employing a 3-amino-benzoyl (Abz) fluorophore, assays were monitored at an excitation wavelength of 310 nn and an emission wavelength of 445 min; the fluorescence plate reader calibrated with 3-amino-benzamide (Fluka). Unless otherwise indicated, all the peptidase substrates were purchased from Bachem UK, St. Helens, Merseyside, UK. Substrates utilizing fluorescence resonance energy transfer methodology (i.e. FRET-based substrates) were synthesized at Incenta Limited using published methods (Atherton & Sheppard, Solid Phase Peptide Synthesis, IRL Press, Oxford, U.K., 1989) and employed Abz (2-aminobenzoyl) as the fluorescence donor and 3-nitro-tyrosine [TyrNO₂)] as the fluorescence quencher (Meldal, M. and Breddam, K., Anal. Biochem., 195, 141-147, 1991). Hydroxyethylpiperazine ethanesulfonate (HEPES), tris-hydroxylmethyl aminomethane (tris) base, bis-tris-propane and all the biological detergents (e.g. CHAPS, zwittergents, etc.) were purchased from CN Biosciences UK, Beeston, Nottinghamshire, U.K. Glycerol was purchased from Amersham Pharmacia Biotech, Little Chalfont, Buckinghamshire, U.K. Stock solutions of substrate or inhibitor were made up to 10 mM in 100% dimethylsulfoxide (DMSO) (Rathburns, Glasgow, U.K.) and diluted as appropriately required. In all cases the DMSO concentration in the assays was maintained at less than 1% (vol./vol.).

Assay protocols were based on literature precedent (Tablel; Barrett, A. J., Rawlings, N. D. and Woessner, J. F., 1998, Handbook of Proteolytic Enzymes, Academic Press, London and references therein) and modified as required to suit local assay protocols. Enzyme was added as required to initiate the reaction and the activity, as judged by the change in fluorescence upon conversion of substrate to product, was monitored over time. All assays were carried out at 25±1° C.

TABLE 1
The enzyme assays described herein were
carried out according to literature precedents.

| Enzyme | Buffer | Substrate | Reference |
|---|---|---|---|
| Cathepsin B | I | Z-Phe-Arg-AMC | a, b |
| Cathepsin H | II | Bz-Phe-Val-Arg-AMC | a, b |
| Cathepsin L | I | Ac-Phe-Arg-AMC | b, c |
| Cathepsin S | I | Boc-Val-Leu-Lys-AMC | c, d |
| Caspase 1 | III | Ac-Leu-Glu-His-Asp-AMC | e |
| Caspase 2 | III | Z-Val-Asp-Val-Ala-Asp-AFC | f |
| Caspase 3 | III | Ac-Asp-Glu-Val-Asp-AMC | g, h |
| Caspase 4 | III | Suc-Tyr-Val-Ala-Asp-AMC | f |
| Caspase 5 | III | Ac-Leu-Glu-His-Asp-AMC | |
| Caspase 6 | III | Ac-Val-Glu-Ile-Asp-AMC | i, j, k |
| Caspase 7 | III | Ac-Asp-Glu-Val-Asp-AMC | |
| Caspase 8 | III | Ac-Ile-Glu-Thr-Asp-AMC | l |
| Caspase 9 | III | Ac-Leu-Glu-His-Asp-AMC | |
| Caspase 10 | III | Ac-Ile-Glu-Thr-Asp-AMC | |
| Cruzipain | IV | D-Val-Leu-Lys-AMC | m, n |
| CPB2.8ΔCTE | XI | Pro-Phe-Arg-AMC | q |
| S. Aureus | I | Abz-Ile-Ala-Ala-Pro- | o |
| Extracellular | | Tyr(NO₂)-Glu-NH₂ | |
| cysteine peptidase | | | |
| Clostripain | | Z-Gly-Gly-Arg-AMC | p |
| FMDV LP | V | Abz-Arg-Lys-Leu-Lys-Gly- | r |
| | | Ala-Gly-Ser-Tyr(NO₂)-Glu- | |
| | | NH₂ | |
| Trypsin | VI | Z-Gly-Gly-Arg-AMC | s |
| Calpain μ | VII | Abz-Ala-Asn-Leu-Gly-Arg-Pro- | t |
| | | Ala-Leu-Tyr(NO₂)-Asp-NH₂ | |
| Calpain m | VIII | Abz-Lys-Leu-Cys(Bzl)-Phe-Ser- | t |
| | | Lys-Gln-Tyr(NO₂)-Asp-NH₂ | |
| Cathepsin K | IX | Z-Phe-Arg-AMC | u |
| Cathepsin X | X | | v, w |

I: 10 mM BTP, pH 6.5 containing 1 mM EDTA, 5 mM 2-mercaptoethanol and 1 mM CaCl₂
II: 10 mM BTP, pH 6.5 containing 1 mM EDTA, 142 mM NaCl, 1 mM DTT, 1 mM CaCl₂, 0.035 mM Zwittergent 3-16
III: 50 mM HEPES pH 7.2, 10% Glycerol, 0.1% CHAPS, 142 mM NaCl, 1 mM EDTA, 5 mM DTT
IV: 100 mM sodium phosphate, pH 6.75 containing 1 mM EDTA and 10 mM L-cysteine
V: 50 mM trisacetate, pH 8.4 containing 1 mM EDTA, 10 mM L-cysteine and 0.25% (w/v) CHAPS
VI: 10 mM HEPES, pH 8.0 containing 5 mM CaCl₂
VII: 10 mM HEPES, pH 7.5 containing 2 mM 2-mercaptoethanol and 100 μM CaCl₂
VIII: 10 mM HEPES, pH 7.5 containing 2 mM 2-mercaptoethanol and 200 μM CaCl₂
IX: 100 mM sodium acetate; pH 5.5 containing 10 mM L-cysteine and 1 mM EDTA
X: 100 mM sodium acetate; pH 5.5 containing 10 mM L-cysteine; 0.05% (w/v) Brij 35 and 1 mM EDTA
XI: 100 mM sodium acetate; pH 5.5 containing 10 mM L-cysteine; 142 mM sodium chloride and 1 mM EDTA
^{a}Barrett, A. J., Biochem. J., 187, 909-912, 1980
^{b}Barrett, A. J. and Kirschke, H., Methods Enzymol., 80, 535-561, 1981
^{c}Quibell, M. and Taylor, S., WO0069855, 2000
^{d}Bromme, D., Steinert, ., Freibe, S., Fittkau, S., Wiederanders, B., and Kirschke, H., Biochem. J., 264, 475-481, 1989
^{e}Rano, T. A., et. al, Chem. Biol., 4, 149, 1997
^{f}Talanian, R. V., et. al., J. Biol. Chem., 272, 9677, 1997
^{g}Lazebnik, Y. A., Kaufmann, S. H., Desnoyers, S., Poirer, G. G. and Earnshaw, W. C., Nature., 371, 768-774, 1994
^{h}Han, Z., et. al., J. Biol. Chem., 272, 13432, 1997
ⁱTakahashi, A., et al., PNAS, 93, 8395, 1996
^{j}Martins, L. M., et. al., J. Biol. Chem., 272, 7421, 1997
^{k}Nagata, S., Cell., 88, 355, 1997
^{l}Harris, J. L., et. al., J. Biol. Chem., 273, 27364, 1998
^{m}Cazzulo, J. J., Cazzulo Franke, M. C., Martinez, J. and Franke de Cazzulo, B. M., Biochim. Biophys. Acta., 1037, 186-191, 1990
ⁿCazzulo, J. J., Bravo, M., Raimondi, A., Engstrom, U., Lindeberg, G. and Hellman, U., Cell Mol. Biol., 42, 691-696, 1996
^{o}Potempa, J., Dubin, A., Korzus, G. and Travis, J., Biochem. J., 263, 2664-2667, 1988
^{p}Kembhavi, A. A., Buttle, D. J., Rauber, P. and Barrett, A. J., FEBS Lett., 283, 277-280, 1991
^{q}Alves, L. C., et. al., Mol. Biochem. Parasitol, 116, 1-9, 2001.
^{r}Guarné, et. al., J. Mol. Biol., 302, 1227-1240, 2000.
^{s}Halfon and Craik, (Barret, Rawlings and Woessner, eds.), in Handbook of Proteolytic Enzymes, Academic Press, London, 12-21, 1998.
^{t}Sasaki, et. al., (1984), J. Biol. Chem., 259, 12489-12494, 1984.
^{u}Bossard, M. J., et. al., , J. Biol. Chem., 21, 12517-12524, 1996
^{v}Santamaria, I., et. al., J. Biol. Chem., 273, 16816-16823, 1998
^{w}Klemencic, J, et al., Eur. J. Biochem., 267, 5404-5412,2000

Trypanosoma Cruzi Cruzipain Peptidase Activity Assays

Wild-type cruzipain, derived from Trypanosoma cruzi Dm28 epimastigotes, was obtained from Dr. Julio Scharfstein (Instituto de Biofisica Carlos Chagas Filho, Universidade Federal do R10 de Janeiro, R10 de Janeiro, Brazil). Activity assays were carried out in 100 mM sodium phosphate, pH 6.75 containing 1 mM EDTA and 10 mM L-cysteine using 2.5 nM enzyme. Ac-Phe-Arg-AMC (K_{M}^{app}≈12 1M) and D-Val-Leu-Lys-AMC (K_{M}^{app}≈4 1M) were used as the substrates. Routinely, Ac-FR-AMC was used at a concentration equivalent to K_{M}^{app} and D-Val-Leu-Lys-AMC was used at a concentration of 25 μM. The rate of conversion of substrate to product was derived from the slope of the increase in fluorescence monitored continuously over time.

Leishmania Mexicana Cysteine Protease B (CPB) Peptidase Activity Assays

Wild-type recombinant CPB without the C-terminal extention (i.e. CPB2.8ACTE; Sanderson, S. J., et. al., Biochem. J., 347, 383-388, 2000) was obtained from Dr. Jeremy Mottram (Wellcome Centre for Molecular Parasitology, The Anderson College, University of Glasgow, Glasgow, U.K.). Activity assays were carried out in 100 mM sodium acetate; pH 5.5 containing 1 mM EDTA; 200 mM NaCl and 10 mM DTT (Alves, L.C., et. al., Mol. Biochem. Parasitol, 116, 1-9, 2001) using 0.25 nM enzyme. Pro-Phe-Arg-AMC (K_{M}^{app}≈38 μM) was used as the substrate at a concentration equivalent to K_{M}^{app}. The rate of conversion of substrate to product was derived from the slope of the increase in fluorescence monitored continuously over time.

Cathepsin Peptidase Activity Assays

Bovine cathepsin S, human cathepsin L, human cathepsin H and human cathepsin B were obtained from CN Biosciences. Recombinant human cathepsin S, human cathepsin K and human cathepsin X were obtained from Dr. Boris Turk (Josef Stefan Institute, Ljubljana, Slovenia). Unless otherwise stated, all peptidase activity assays were carried out in 10 mM bis-tris-propane (BTP), pH 6.5 containing 1 mM EDTA, 5 mM 2-mercaptoethanol and 1 mM CaCl₂. Human cathepsin H activity assays were carried out in 10 mM BTP pH 6.5, 142 MM NaCl2 1 mM CaCl₂, 1 mM EDTA, 1 mM DTr, 0.035 mM Zwittergent 3-16. Human cathepsin K assays were carried out in 100 mM sodium acetate; pH 5.5 contaiing 20 mM L-cysteine and 1 mM EDTA (Bossard, M. J., et. al., J. Biol. Chem., 2., 12517-12524, 1996). Human cathepsin X assays were carried out in 100 mM sodium acetate; pH 5.5 containing 20 mM L-cysteine; 0.05% (w/v) Brij 35 and 1 mM EDTA (Santamaria, I., et. al., J. Biol. Chem., 273 16816-16823, 1998; Klemencic, J, et al., Eur. J. Biochem., 267 5404-5412, 2000). The final enzyme concentrations used in the assays were 0.5 nM bovine cathepsin S, 1 nM cathepsin L, 0.1 nM cathepsin B, 0.25 nM Cathepsin K; 1 nM cathepsin X and 10 nM cathepsin H. For the inhibition assays, the substrates used for cathepsin S, cathepsin L, cathepsin B, cathepsin K and cathepsin H were boc-Val-Leu-Lys-AMC (K_{M}^{app}≈30 μM), Ac-Phe-Arg-AMC (K_{M}^{app}≈20 μM), Z-Phe-Arg-AMC (K_{M}^{app}≈40 μM), Z-Leu-Arg-AMC (K_{M}^{app}≈2 μM); Bz-Phe-Val-Arg-AMC (K_{M}^{app}≈150 μM) respectively. In each case the substrate concentration used in each assay was equivalent to the K_{M}^{app}. The rate of conversion of substrate to product was derived from the slope of the increase in fluorescence monitored continuously over time.

Trypsin Peptidase Activity Assays

Human pancreatic trypsin (iodination grade; CN Biosciences) activity assays were carried out in 10 mM HEPES, pH 8.0 containing 5 mM CaCl₂ using 0.1 nM trypsin. For the inhibition assays, Z-Gly-Gly-Arg-AMC (K_{M}^{app}≈84 μM) was used as the substrate at a concentration equivalent to K_{M}^{app}. The rate of conversion of substrate to product was derived from the slope of the increase in fluorescence monitored continuously over time.

Clostripain Peptidase Activity Assays

Clostripain (Sigrma) activity assays were carried out in 10 mM BTP, pH 6.5 containing 1 mM EDTA, 5 mM 2-mercaptoethanol and 1 mM CaCl₂ using 0.3 nM enzyme. For the inhibition assays, Z-Gly-Gly-Arg-AMC (K_{M}^{app}≈100 μM) was used as the substrate at a concentration equivalent to K_{M}^{app}. The rate of conversion of substrate to product was derived from the slope of the increase in fluorescence monitored continuously over time.

Calpain Peptidase Activity Assays

Calpain (human erythrocyte μ-calpain and porcine kidney m-calpain; CN Biosciences) activity assays were carried out in 10 mM HEPES, pH 7.5 containing 2 mM 2-mercaptoethanol and CaCl₂ using 25 nM of either enzyme (Sasaki, et. al., J. Biol. Chem., 259, 12489-12494, 1984). For i-calpain inhibition assays, the buffer contained 100 μM CaCl₂ and Abz-Ala-Asn-Leu-Gly-Arg-Pro-Ala-Leu-Tyr(NO₂)-Asp-NH₂ (K_{M}^{app}≈20 μM; Incenta Limited) was used as the substrate. For m-calpain inhibition assays, the assay buffer contained 200 μM CaCl₂ and Abz-Lys-Leu-Cys(Bzl)-Phe-Ser-Lys-Gln-Tyr(NO₂)-Asp-NH₂ (K_{M}^{app}≈22 μM; Incenta Limited) was used as the substrate. In both cases the substrate concentration employed in the assays was equivalent to the K_{M}^{app}. The rate of conversion of substrate to product was derived from the slope of the increase in fluorescence monitored continuously over time.

Extracellular S. Aureus V8 Cysteine Peptidase (Staphylopain) Peptidase Activity Assays

S. aureus V8 was obtained from Prof S. Arvidson, Karolinska Institute, Stockholm, Sweden. Extracellular S. aureus V8 cysteine peptidase (staphylopain) activity assays were carried out using partially purified S. aureus V8 culture supernatant (obtained from Dr. Peter Lambert, Aston University, Birmingham, U.K.). Activity assays were carried out in 10 mM BTP, pH 6.5 containing 1 mM EDTA, S mM 2-mercaptoethanol and 1 mM CaCl₂ using two-times diluted partially purified extract. For the inhibition assays, Abz-le-Ala-Ala-Pro-Tyr(NO₂)-Glu-NH₂ (K_{M}^{app}≈117 μM; Incenta Limited) was used as the substrate at a concentration equivalent to K_{M}^{app}. The rate of conversion of substrate to product was derived from the slope of the increase in fluorescence monitored continuously over time.

Foot-and-Mouth Disease Leader Peptidase (FMDV-LP) Activity Assays

Recombinant wild-type FMDV-LP was obtained from Dr. Tim Skem (Institut fur Medizinische Biochemie, Abteilung flir Biochemie, Universtit Wien, Wien, Austria). Activity assays were carried out in 50 mM trisacetate, pH 8.4 containing 1 mM EDTA, 10 mM L-cysteine and 0.25% (w/v) CHAPS using 10 nM enzyme. For the inhibition assays, Abz-Arg-Lys-Leu-Lys-Gly-Ala-Gly-Ser-Tyr(NO₂)-Glu-NH₂ (K_{M}^{app}≈51 μM, Incenta Limited) was used as the substrate at a concentration equivalent to K_{M}^{app}. The rate of conversion of substrate to product was derived from the slope of the increase in fluorescence monitored continuously over time.

Caspase Peptidase Activity Assays

Caspases 1-10 were obtained from CN Biosciences or BioVision Inc. (Mountain View, Calif., USA) and all assays were carried out in 50 mM HEPES; pH 7.2, 10% (v/v) glycerol, 0.1% (w/v) CHAPS, 142 mM NaCl, 1 mM EDTA, 5 mM dithiothreitol ( )Tl) using 0.1-1 U per assay. For caspase 1, Ac-Leu-Glu-His-Asp-AMC was used as the substrate; for caspase 2, Z-Val-Asp-Val-Ala-Asp-AFC was used as the substrate; for caspase 3, Ac-Asp-Glu-Val-Asp-AMC was used as the substrate; for caspase 4, Suc-Tyr-Val-Ala-AspAMC was used as the substrate; for caspase 5, Ac-Leu-Glu-His-Asp-AMC was used as the substrate; for caspase 6, Ac-Val-Glu-Ile-Asp-AMC was used as the substrate; for caspase 7, Ac-Asp-Glu-Val-Asp-AMC was used as the substrate; for caspase 8, Ac-fle-Glu-Thr-Asp-AMC was used as the substrate; for caspase 9, Ac-Leu-Glu-His-Asp-AMC was used as the substrate; for caspase 10, Ac-Ile-Glu-Thr-Asp-AMC was used as the substrate (Nicholson, D. W. and Thomberry, N. A., TIBS, 2, 299-306, 1997; Stennicke, H. R. and Salvesen, G. S., J. Biol. Chem., 272(41), 25719-25723, 1997; Talanian, R. V., et. al., J. Biol. Chem., 272(15), 9677-9682, 1997; Wolf B. B. and Green, D. R., J. Biol. Chem., 274(29), 20049-20052, 1999). The rate of conversion of substrate to product was derived from the slope of the increase in fluorescence monitored continuously over time.

Measurement of the Apparent Macroscopic Binding (Michaelis) Constants (K_{M}^{app}) for Substrates

The apparent macroscopic binding constant (K_{M}^{app}) for each substrate was calculated, from the dependence of enzyme activity as a function of substrate concentration. The observed rates were plotted on the ordinate against the related substrate concentration on the abscissa and the data fitted by direct regression analysis (Prism v 3.02; GraphPad, San Diego, USA) using Equation 1 (Cornish-Bowden, A. Fundamentals of enzyme kinetics Portland Press; 1995, 93-128.).

$${v}_{i} = \frac{{V}_{max}^{app} \cdot [ {S}_{o} ]}{[ {S}_{o} ] + {K}_{M}^{app}} \qquad ( 1 )$$

In Equation 1 ‘v₁’ is the observed initial rate, ‘V_{mzx}^{app}’ is the observed maximum activity at saturating substrate concentration, ‘K_{M}^{app}’ is the apparent macroscopic binding (Michaelis) constant for the substrate, ‘[Sₒ]’ is the initial substrate concentration.

Measurement of the Inhibition Constants

The apparent inhibition constant (Kᵢ) for each compound was determined on the basis that inhibition was reversible and occurred by a pure-competitive mechanism. The Kᵢ values were calculated, from the dependence of enzyme activity as a function of inhibitor concentration, by direct regression analysis (Prism v 3.02) using Equation 2 (Cornish-Bowden, A., 1995.).

$${v}_{i} = \frac{{V}_{max}^{app} \cdot [ S ]}{[ S ] + \{ {K}_{M}^{app} \cdot ( [ I ] / {K}_{i} ) \}} \qquad ( 2 )$$

In Equation 2 ‘v₁’ is the observed residual activity, ‘Vₘₐₓ^{app}’ is the observed maximum activity (i.e. in the absence of inhibitor), ‘K_{M}^{app}’ is the apparent macroscopic binding (Michaelis) constant for the substrate, ‘[S]’ is the initial substrate concentration, ‘Kᵢ’ is the apparent dissociation constant and ‘[I]’ is the inhibitor concentration.

In situations where the apparent dissociation constant (Kᵢ^{app}) approached the enzyme concentrations, the Kᵢ^{app} values were calculated using a quadratic solution in the form described by Equation 3 (Morrison, J. F. Trends Biochem. Sci., 7, 102-105, 1982; Morrison, J. F. Biochim. Biophys. Acta, 185, 269-286, 1969; Stone, S.R. and Hofsteenge, J. Biochemistry, 25, 4622-4628, 1986).

$${v}_{i} = \frac{F \{ {E}_{o} - {I}_{o} - {K}_{i}^{app} + \sqrt{{( {E}_{o} - {I}_{o} - {K}_{i}^{app} )}^{2} + 4 \cdot {K}_{i}^{app} \cdot {E}_{o}} \}}{2} \qquad ( 3 )$$

Kᵢ^{app}=Kᵢ(1+[Sₒ]/K_{M}^{app}) (4)

In Equation 3 ‘vᵢ’ is the observed residual activity, ‘F’ is the difference between the maximum activity (i.e. in the absence of inhibitor) and minimum enzyme activity, ‘Eₒ’ is the total enzyme concentration, ‘Kᵢ^{app}’ is the apparent dissociation constant and ‘Iₒ’ is the inhibitor concentration. Curves were fitted by non-linear regression analysis (Prism) using a fixed value for the enzyme concentration. Equation 4 was used to account for the substrate kinetics, where ‘Kᵢ’ is the inhibition constant, ‘[Sₒ]’ is the initial substrate concentration and ‘K_{M}^{app}’ is the apparent macroscopic binding (Michaelis) constant for the substrate (Morrison, 1982).

The Second-Order Rate of Reaction of Inhibitor with Enzyme

Where applicable, the concentration dependence of the observed rate of reaction (k_{obs}) of each compound with enzyme was analysed by determining the rate of enzyme inactivation under pseudo-first order conditions in the presence of substrate (Morrison, J. F., TIBS, 102-105, 1982; Tian, W. X. and Tsou, C. L., Biochemistry, 21, 1028-1032, 1982; Morrison, J. F. and Walsh, C. T., from Meister (Ed.), Advances in Enzymol., 61, 201-301, 1988; Tsou, C. L., from Meister (Ed.), Advances in Enzymol., 61, 381-436, 1988;). Assays were carried out by addition of various concentrations of inhibitor to assay buffer containing substrate. Assays were initiated by the addition of enzyme to the reaction mixture and the change in fluorescence monitored over time. During the course of the assay less than 10% of the substrate was consumed.

$$F = {v}_{s} t + \frac{( {v}_{o} - {v}_{s} ) ⌊ 1 - {ⅇ}^{( {k}_{obs} \cdot t )} ⌋}{{k}_{obs}} + D \qquad ( 5 )$$

The activity fluorescence progress curves were fitted by non-linear regression analysis (Prism) using Eq. 5 (Morrison, 1969; Morrison, 1982); where ‘F’ is the fluorescence response, ‘t’ is time, ‘vₒ’ is the initial velocity, ‘vₛ’ is the equilibrium steady-state velocity, ‘k_{obs}’ is the observed pseudo first-order rate constant and ‘D’ is the intercept at time zero (ie. the ordinate displacement of the curve). The second order rate constant was obtained from the slope of the line of a plot of k_{obs} versus the inhibitor concentration (i.e. k_{obs}[T]). To correct for substrate kinetics, Eq. 6 was used, where ‘[Sₒ]’ is the iniitial substrate concentration and ‘K_{M}^{app}’ is the apparent macroscopic binding (Michaelis) constant for the substrate.

$${k}_{inact} = \frac{{k}_{obs} ( 1 + [ {S}_{o} ] / {K}_{M}^{app} )}{[ I ]} \qquad ( 6 )$$

Compounds of the invention were tested by the above described assays and observed to exhibit cruzipain inhibitory activity or inhibitory activity against an alternative CA C1 cysteine protease with an in vitro Ki inhibitory constant of less than or equal to 100 μM. Exemplary inhibition data for a number of example compounds of the invention are given in table 2.

TABLE 2
Exemplary inhibition data (Ki expressed as μM).

| | | Bovine | Human | Human |
|---|---|---|---|---|
| EXAMPLE N^{o} | Cruzipain | Cathepsin S | Cathepsin L | Cathepsin K |
| 6 | <2 | >50 | >50 | >100 |
| 19 | >50 | <2 | >50 | >50 |
| 27 | >20 | >50 | <5 | >100 |

1. A compound according to general formula (I): 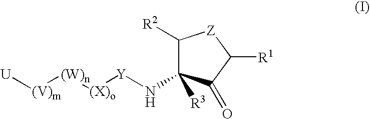
wherein:
R¹=C₀₋₇-alkyl (when C=0, R¹ is simply hydrogen), C₃₋₆-cycloallyl or Ar—C₀₋₇-alkyl (when C=0, R¹ is simply an aromatic moiety Ar);
R²=C₀₋₇-alkyl, C₃₋₆-cycloalkyl or Ar—CO-alkyl;
R³=C₁₋₇-alkyl, C₃₋₆-cycloalkyl or Ar—C₀₋₇-alkyl;
Y═CR⁴R⁵—CO where R⁴, R⁵ are independently chosen from C₀₋₇-alkyl, C₃₋₆-cycloalkyl and Ar—C₀₋₇-alkyl;
(X)ₒ=CR⁶R⁷, where R⁶ and R⁷ are independently chosen from C₀₋₇-alkyl, C₃₋₆-cycloalkyl and Ar—C₀₋₇-alkyl and o is a number from zero to three;
(n=O, S, C(O), S(O) or S(O)₂ or NR⁸, where R⁸ is chosen from C₀₋₇-alkyl, C₃₋₆-cycloalkyl and Ar—C₀₋₇-alkyl and n is zero or one;
(V)ₘ═C(O), C(S), S(O), S(O)₂, S(O)₂NH, OC(O), NHC(O), NHS(O), NHS(O)₂, OC(O)NH, C(O)NH or CR⁹R¹⁰, where R⁹ and R¹⁰ are independently chosen from C₀₋₇-alkyl, C₃₋₄-cycloalkyl, Ar—C₀₋₇-alkyl and m is a number from zero to three, provided that when m is greater than one, (V)ₘ contains a maximum of one carbonyl or sulphonyl group;
Z=O (in which case compounds of general formula (I) may be named as (2-alkyl-3-alkyl-4-oxo-tetrahydrofuran-3-yl)amides, S (in which case compounds of general formula (I) may be named as (2-alkyl-3-alkyl-4-oxo-tetrahydrothiophen-3-yl)amides or CH₂ (in which case compounds of general formula (I) may be named as and (1-alkyl-2-alkyl-5-oxocyclopentyl)amides;
U=a stable 5- to 7-membered monocyclic or a stable 8- to 11-membered bicyclic ring which is either saturated or unsaturated and which includes zero to four heteroatoms (as detailed below): 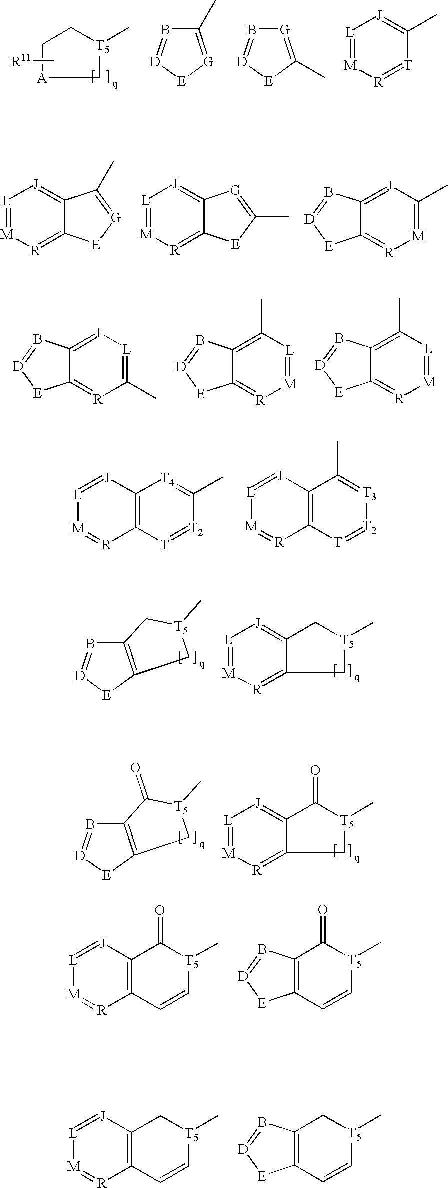
wherei R¹¹ is chosen from:
C₀₋₇-alkyl, C₃₋₄-cycloalkyl, Ar—C₀₋₇-alkyl, halogen, O—C₀₋₇-alkyl, O—C₃₋₄-cycloalkyl, O—Ar—C₀₋₇-alkyl, S—C₀₋₇-alkyl, S—C₃₋₄-cycloalkyl, S—Ar—C₀₋₇-alkyl, NH—CO-alkyl, NH—C₃-cycloalkyl, NH—Ar—C₀₋₇-alkyl, N—(C₀₋₇-alkyl)₂, N—(C₃₋₆-cycloalkyl)₂ and N—(Ar—C₀₋₇-alkyl)₂; or, when part of a group CHR¹¹ or CR¹¹, R¹¹ may be halogen;
A is chosen from:
CH₂, CHR¹¹, O, S and NR¹², where R¹¹ is as defined above and where R¹² is chosen from C₀₋₇-alkyl, C₃₋₆-cycloalkyl and Ar—C₀₋₇-alkyl;
B, D and G are independently chosen from:
CR¹¹, where R¹¹ is as defined above, or N;
E is chosen from:
CH₂, CHR¹¹, O, S and NR¹², where R¹¹ and R¹² are defined as above;
J, L, M, R, T, T₂, T₃ and T₄ are independently chosen from:
CR¹¹ and N, where R¹¹ is as defined above;
T₅ is chosen from:
CH or N;
q is a number from one to three, thereby defining a 5-, 6- or 7-membered

2. A compound as claimed in claim 1 wherein R¹ comprises C₀₋₇-alkyl or Ar—C₀₋₇-alkyl.

3. A compound as claimed in claim 2 wherein R¹ is selected from hydrogen or one of the following moieties:

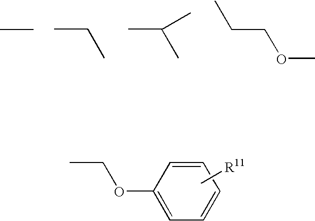

4. A compound as claimed in any one of claims 1 to 3 wherein R² is C₁₋₇-alkyl or Ar—C₀₋₇-alkyl.

5. A compound as claimed in claim 4 wherein R² is selected from one of the following moieties: 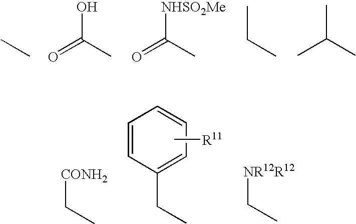
wherein R¹¹ and R¹² are as defined previously.

6. A compound as claimed in any one of claims 1 to 4 wherein R³ is a simple allyl or arylalkyl group.

7. A compound as claimed in any one of claims 1 to 6 wherein Y is CR⁴R⁵CO where R⁴, R⁵ are selected from C₀₋₇-alkyl, C₃₋₆-cycloalkyl or Ar—C₀₋₇-alkyl.

8. A compound as claimed in claim 7 where Y is selected from one of the following moieties: 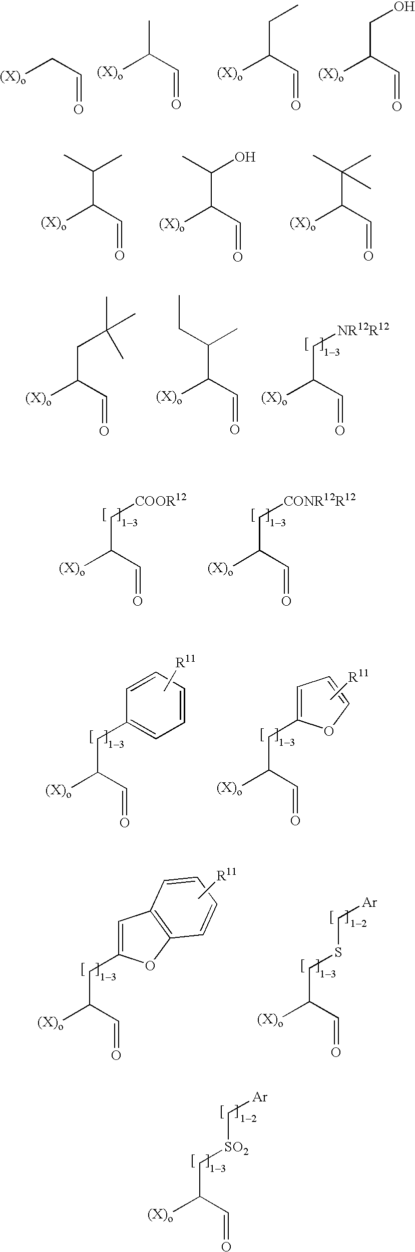
wherein R¹¹, R¹² and Ar are as defined above.

9. A compound as claimed in any one of claims 1 to 8 wherein Y is CHR⁵CO where R⁵ is Ar—CH₂—, where the aromatic ring is an optionally substituted phenyl or monocyclic heterocycle

10. A compound as claimed in any one of claims 1 to 8 wherein Y is CHR⁵CO where R⁵ is a simple branched alkyl group or a straight heteroalkyl chain.

11. A compound as claimed in any one of claims 1 to 8 wherein Y is CHR⁵CO where R⁵ comprises cyclohexylmethyl.

12. A compound as claimed in any one of claims 1 to 8 wherein Y is selected from the following: 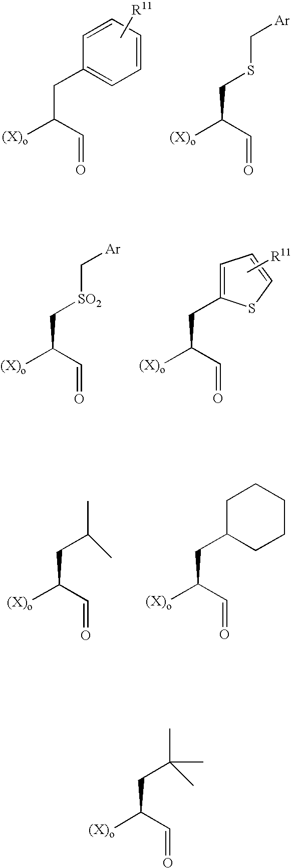
wherein R¹¹ and Ar are as defined previously

13. A compound as claimed in any one of claims 1 to 12 wherein, in the group (X)M, X is CR⁶R⁷ and each of R⁶ and R⁷ is selected from C₀₋₇-alkyl or Ar—C₀₋₇-alkyl

14. A compound as claimed in any one of claims 1 to 13, wherein (X). is one of the following moieties: 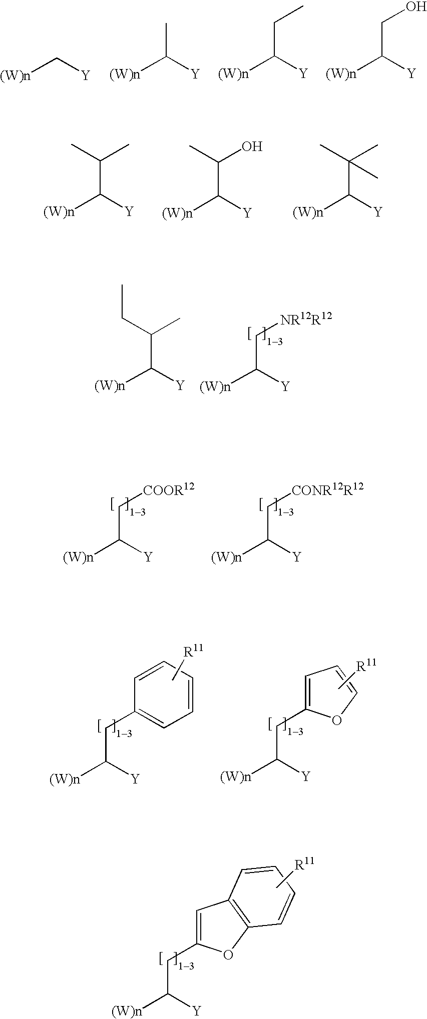
wherein R¹¹ and R¹² are as defined previously.

14. A compound as claimed in any one of claims 1 to 12, wherein (X)ₒ a simple alkyl group and where o=0 or 1.

15. A compound as claimed in any one of claims 1 to 14 wherein, in the group (W)ₙ:
W is O, S, SO₂, SO, C(O) or NR⁸, where R⁸ is C₀₋₄-alkyl; and n is 0 or 1.

16. A compound as claimed in any one of claims 1 to 15 wherein, in the group (W)ₙ:
W is O, S, SO₂, C(O) or NH where n is 0 or 1.

17. A compound as claimed in any one of claims 1 to 16 wherein, in the group (V)ₘ:
V is C(O), C(O)NH or CHR¹⁰, where R¹⁰ is C₀₋₄-alkyl; and
m is 0 or 1.

18. A compound as claimed in any one of claims 1 to 17 wherein the combination (V)ₘ and (W)ₘ is one of the following:

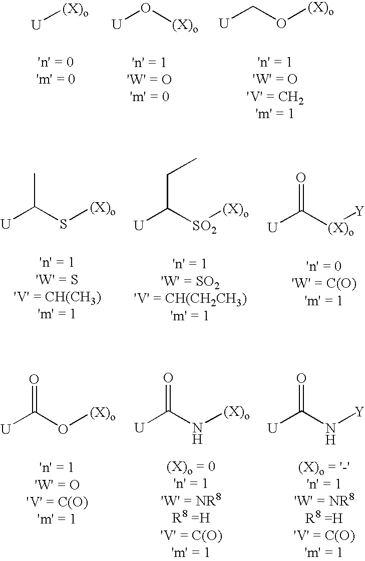

19. A compound as claimed in claim 18, wherein the combination (V)ₘ and (W)ₘ is one of the first eight structures depicted in claim 18.

20. A compound as claimed in claim 18, wherein the combination (V)ₘ and (W)ₘ is the ninth structure depicted in claim 18.

21. A compound as claimed in any one of claims 1 to 17 wherein the combination (X)ₒ, (V)ₘ and (W)ₘ is one of the following:

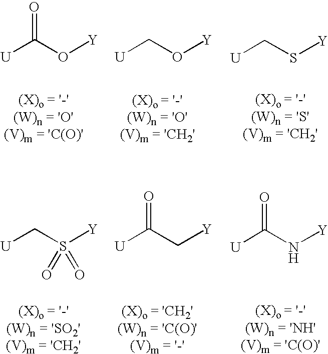

22. A compound as claimed in any one of claims 1 to 21 wherein U comprises an optionally substituted 5- or 6-membered saturated or unsaturated heterocycle or an optionally substituted saturated or unsaturated 9- or 10-membered heterocycle.

23. A compound as claimed in claim 22 wherein U comprises one of the following: 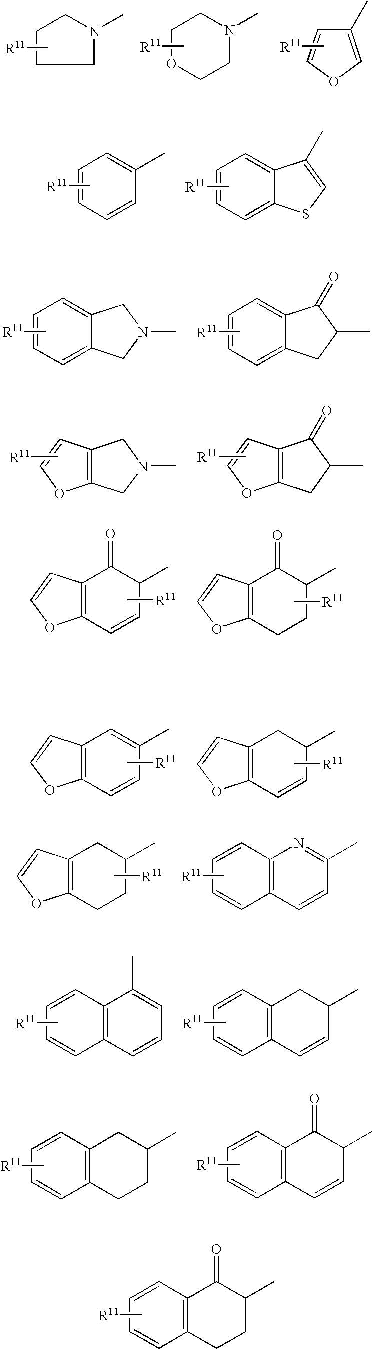
wherein R¹¹ is as defined previously.

24. A compound as claimed in any one of claims 1 to 21 wherein U comprises a bulky alkyl or aryl group at the para position of an aryl Ar.

25. A compound as claimed in any one of claims 1 to 23 wherein U comprises a meta or para-biaryl Ar-Ar, where Ar is as previously defined.

26. A compound as claimed in any one of claims 1 to 21 wherein U comprises a 6,6 or 6,5 or 5,6-fused aromatic ring.

27. A compound as claimed in any one of claims 1 to 21, wherein U represents a group: 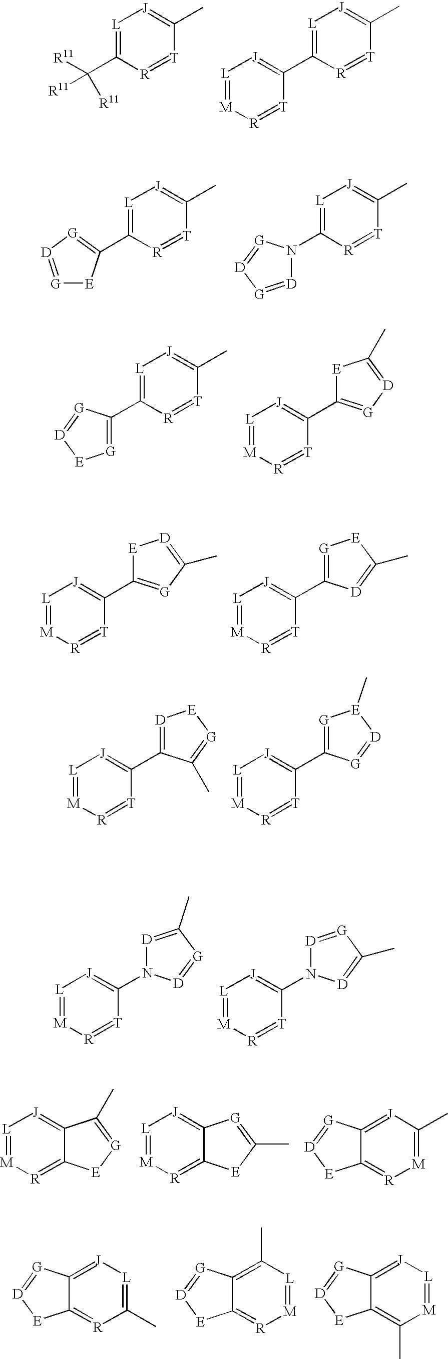 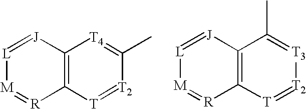
wherein R¹¹, D, E, G, J, L, M, R, T, T₂, T₃ and T₄ are as defined previously.

28. A compound as claimed in any one of claims 1 to 21, wherein U represents a group 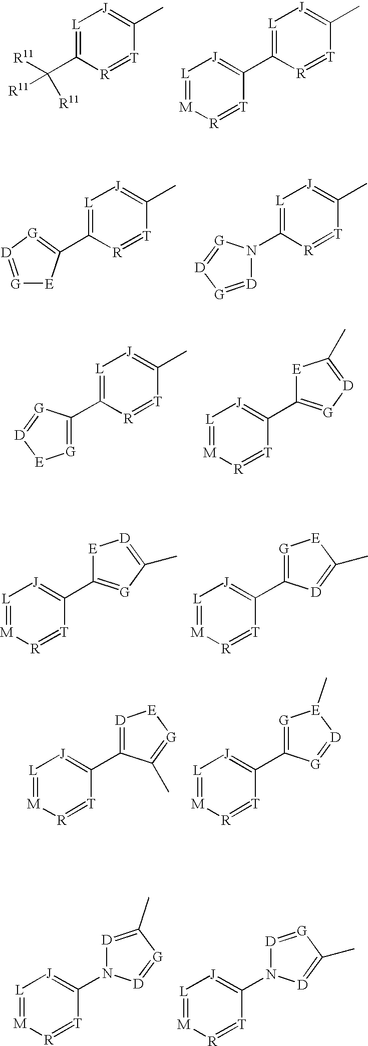
wherein R¹¹, D, E, G, J, L, M, R and T are as defined previously.

29. A compound as claimed in any one of claims 1 to 21, wherein U represents a group 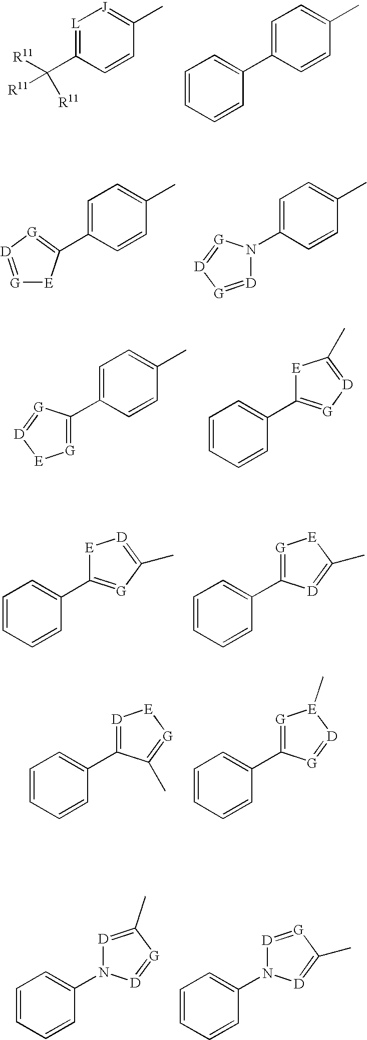
wherein R¹¹, D, E, G, J and L are as defined previously.

30. A method of validating a known or putative cysteine protease inhibitor as a therapeutic target, the method comprising:
(a) assessing the in vitro binding of a compound as claimed in any one of claims 1 to 29 to an isolated known or putative cysteine protease, providing a measure of ‘potency’; and optionally, one or more of the steps of:
(b) assessing the binding of a compound as claimed in any one of claims 1 to 29 to closely related homologous proteases of the target and general house-keeping proteases (e.g. trypsin) to provides a measure of ‘selectivity’;
(c) monitoring a cell-based functional marker of a particular cysteine protease activity, in the presence of a compound as claimed in any one of claims 1 to 29; and
(d) monitoring an animal model-based functional marker of a particular cysteine protease activity, in the presence of a compound as claimed in any one of claims 1 to 29.

31. The use of a compound as claimed in any one of claims 1 to 29 in the validation of a known or putative cysteine protease inhibitor as a therapeutic target.

32. A compound as claimed in any one of claims 1 to 29 for use in medicine, especially for preventing or treating diseases in which the disease pathology may be modified by inhibiting a cysteine protease.

33. The use of a compound as claimed in any one of claims 1 to 29 in the preparation of a medicament for preventing or treating diseases in which the disease pathology may be modified by inhibiting a cysteine protease.

34. A compound as claimed in any one of claims 1 to 29 for use in preventing or treating Chagas' disease.

35. A compound as claimed in any one of claims 24 to 29 for use in preventing or treating Chagas' disease.

36. The use of a compound as claimed in any one of claims 1 to 29 in the preparation of a medicament for preventing or treating Chagas' disease.

37. The use of a compound as claimed in any one of claims 24 to 29 in the preparation of a medicament for preventing or treating Chagas' disease.

38. A pharmaceutical or veterinary composition comprising one or more compounds as claimed in any one of claims 1 to 29 and a pharmaceutically or veterinarily acceptable carrier.

39. A process for the preparation of a pharmaceutical or veterinary composition as claimed in claim 38, the process comprising bringing the active compound(s) into association with the carrier, for example by admixture.
